(19) United States
(12) Patent Application Publication
(10) Pub. No.: US 2026/0176649 A1
(43) Pub. Date: Jun. 25, 2026

(54) COMPOSITIONS AND METHODS TO INCREASE RESISTANCE TO PHYTOPHTHORA SOJAE IN SOYBEAN

(71) Applicants: CORTEVA AGRISCIENCE LLC, Indianapolis, IN (US); PURDUE RESEARCH FOUNDATION, West Lafayette, IN (US)

(72) Inventors: Jianxin MA, West Lafayette, IN (US); Rajat AGGARWAL, Indianapolis, IN (US); Liyang CHEN, West Lafayette, IN (US); Weidong WANG, West Lafayette, IN (US); Oswald CRASTA, Banford, CT (US); Jonathan MYRVOLD, Indianapolis, IN (US)

(21) Appl. No.: 19/546,028

(22) Filed: Feb. 20, 2026

Related U.S. Application Data
(62) Division of application No. 18/266,871, now Pat. No. 12,577,577.
(60) Provisional application No. 63/126,283, filed on Dec. 16, 2020.
(60) Provisional application No. 63/154,913, filed on Mar. 1, 2021.

(51) Int. Cl. C12N 15/82; C12N 9/22; C12N 15/11; C12Q 1/6869; C12Q 1/6895

(52) U.S. Cl. CPC C12N 15/8279; C12N 9/22; C12N 15/11; C12N 15/8213; C12N 15/8282; C12Q 1/6869; C12Q 1/6895; C12N 2310/20; C12N 2800/80; C12Q 2600/13; C12Q 2600/156

(57) ABSTRACT

Provided are compositions comprising polynucleotides encoding Rps genes, and soybean plants or soybean seeds comprising the compositions and exhibiting resistance to Phytophthora. Additionally, various methods for employing the polynucleotides to increase resistance to Phytophthora are also provided herein.

### CROSS REFERENCE TO RELATED APPLICATIONS

This application is a divisional application of U.S. application Ser. No. 18/266,871, filed on Jun. 13, 2023, which is a U.S. national counterpart application of International Application no. PCT/US2021/063556, filed on Dec. 15, 2021 which claims priority to U.S. Provisional Application Ser. No. 63/126,283, filed on Dec. 16, 2020 and U.S. Provisional Application Ser. No. 63/154,913, filed on Mar. 1, 2021, respectively, the disclosures of which are expressly incorporated herein.

### REFERENCE TO SEQUENCE LISTING SUBMITTED ELECTRONICALLY

The official copy of the sequence listing is submitted electronically via EFS-Web as an XML formatted sequence listing with a file name: 76751438620.xml created on Feb. 19, 2026 and having a size of 143,000 bytes and is filed concurrently with the specification. The sequence listing comprised in this XML formatted document is part of the specification and is herein incorporated by reference in its entirety.

### FIELD

This disclosure relates generally to the field of molecular biology.

### BACKGROUND

Soybean diseases are major threat for soybean production, resulting in yield losses and decrease in grain quality. Phytophthora root and stem root (PRSR), caused by the soil-borne oomycete pathogen Phytophthora sojae, is one of the top five most destructive diseases leading to soybean yield loss. From 1996 to 2016, the total estimated economic loss due to PRSR was 7.4 billion USD in the United States and ranked as the third most severe soybean disease after soybean cyst nematode (SCN) and seedling disease (Bandara A Y et al. 2019). Resistance to Phytophthora infection is conditioned by naturally occurring variation at the Resistance to Phytophthora sojae (Rps) loci. As races of Phytophthora in the fields shift, previously effective resistance sources are breaking down, causing damage and compromised yields in grower fields.

Accordingly, there is a need to develop new compositions and methods for conferring resistance to Phytophthora sojae. This disclosure provides such compositions and methods.

### SUMMARY

In accordance with one embodiment an isolated gene construct is provided comprising a heterologous regulatory sequence operably linked to a polynucleotide encoding a polypeptide comprising an amino acid sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to any one of SEQ ID NOs: 2 or 4. In one embodiment the heterologous regulatory element is a heterologous promoter. In one embodiment the isolated gene construct comprises a heterologous promoter operably linked to a polynucleotide sequence encoding a polypeptide comprising SEQ ID NOs: 2 or 4. In one embodiment the isolated gene construct comprises a heterologous promoter operably linked to a polynucleotide sequence having at least 95% sequence identity with SEQ ID NO: 1 or 3.

In one embodiment soybean plants or soybean seeds are provided comprising a targeted genetic modification increasing expression of a polynucleotide encoding a polypeptide comprising an amino acid sequence having at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity to any one of SEQ ID NOs: 2 or 4 as compared to a control plant not comprising the targeted genetic modification. In certain embodiments, the soybean plant or a plant grown from the soybean seed comprising the targeted genetic modification has improved resistance to Phytophthora infection as compared to the control plant. In certain embodiments, the targeted genetic modification introduces a polynucleotide sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of SEQ ID NOs: 1 or 3, optionally wherein said polynucleotide is operably linked to a heterologous promoter.

Further provided are plants grown from seed whose cells comprise a polynucleotide encoding a polypeptide having at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 2 or 4, and methods of plant breeding comprising crossing such soybean plants with a second soybean plant to produce a progeny seed. In certain embodiments, the second soybean plant is susceptible to the at least one race of Phytophthora. In certain embodiments, the progeny seed comprises the targeted genetic modification and a plant produced from the seed has increased resistance to at least one race of Phytophthora.

Also provided are methods for generating a Phytophthora resistant soybean plant comprising introducing in a regenerable soybean plant cell a polynucleotide encoding a polypeptide comprising an amino acid sequence having at least 80% sequence identity to any one of SEQ ID NOs: 2 or 4, and generating the plant wherein the plant expresses the polynucleotide and has increased resistance to Phytophthora as compared to a control plant not expressing the polynucleotide. In certain embodiments, the regenerable plant cell is isolated from a soybean plant susceptible to at least one race of Phytophthora and the plant generated has increased resistance to the at least one race of Phytophthora. In certain embodiments, the polynucleotide is introduced in the regenerable soybean plant cell using a targeted genetic modification

Further provided are methods for generating a Phytophthora resistant soybean plant comprising introducing in a regenerable soybean plant cell a targeted genetic modification increasing the expression of a polynucleotide encoding a polypeptide comprising an amino acid sequence having at least 80% sequence identity to any one of SEQ ID NOs: 2 or 4, optionally at least 95% sequence identity to any one of SEQ ID NOs: 2 or 4, and generating the plant wherein the plant has increased expression of the polynucleotide and increased resistance to Phytophthora infection as compared to a control plant not comprising the targeted genetic modification, optionally compared to the parent plant that was used to generate the modified plant that exhibits increased expression of a polynucleotide encoding a polypeptide comprising an amino acid sequence having at least 80% sequence identity to any one of SEQ ID NOs: 2 or 4.

Also provided are soybean plants or seed comprising a recombinant DNA construct comprising a polynucleotide encoding a polypeptide comprising an amino acid sequence that is at least 80% identical to any one of SEQ ID NOs: 2 or 4, wherein the soybean seed or soybean plant has increased expression of the polynucleotide as compared to a control plant not comprising the polynucleotide, optionally wherein the DNA construct comprises a heterologous promoter operably linked to a polynucleotide encoding a polypeptide comprising an amino acid sequence that is at least 80% identical to any one of SEQ ID NOs: 2 or 4.

Further provided are methods for increasing resistance to Phytophthora infection in a soybean plant comprising expressing in a regenerable soybean plant cell a recombinant DNA construct comprising a polynucleotide encoding a polypeptide comprising an amino acid sequence having at least 80% sequence identity to any one of SEQ ID NOs: 2 or 4, and generating the plant wherein the plant has increased expression of the polynucleotide and increased resistance to Phytophthora infection as compared to a control plant not comprising the recombinant DNA construct.

Also provided are methods for identifying a soybean plant that displays increased resistance to Phytophthora, comprising detecting in a soybean plant or seed thereof a polynucleotide sequence encoding a polypeptide comprising an amino acid sequence that is at least 80% identical to any one of SEQ ID NOs: 2 or 4 or detecting in a soybean plant or seed thereof at least one allele of a marker locus associated with Rps11, Rps2b (also known as Rps2cas), Rps15 (also known as Rps2f), or Rps14 (also known as Rps1f).

### BRIEF DESCRIPTION OF THE DRAWINGS AND THE SEQUENCE LISTING

The disclosure can be more fully understood from the following detailed description and the accompanying drawings and Sequence Listing, which form a part of this application, which are incorporated herein by reference.

FIG. 1 depicts the expression level of the twelve NBS-LRR genes across the Rps11 locus in the Rps11 donor line.

FIG. 2 provides experimental results depicting the expression profile of the NBS-LRR genes in 9 recombinants. The y-axis is the expression value in Fragments per Kilobase per Million mapped reads (FPKM) and the x-axis is the code of twelve NBS-LRR genes at Rps11 locus. The phenotype (i.e., susceptible or resistant) of each recombinant is listed and recombinants with the same expression pattern are combined together.

FIG. 3 provides experimental results showing the initial mapping of Rp2cas from the bulked segregant analysis (BSA). Triangles represent the genotypes of the susceptible bulk at the SNP sites between the two parental lines, while circles are genotypes of the resistant bulk at the same set of SNP sites. The x-axis shows the physical positions of these SNP sites along chromosome 16. On the y-axis, the T1 position represents the homozygous genotype detected in the susceptible bulk, and the T2 position indicates the heterozygous genotypes that were detected in susceptible or/and resistant bulks. Dotted vertical lines a and b define the two boundaries of the Rps2b region from initial BSA mapping.

FIG. 4 provides the genetic and physical map of the Rps2b region. The left bar (a) provides the genetic map of the Rps2b region according to linkage analysis. SSR markers are listed on the left side and the genetic distance (centimorgan, cM) between adjacent markers are shown on the right side of the map. The middle bar (b) provides the physical positions of molecular markers on chromosome 16 based on soybean reference genome (Wms82 v2.1). Both genetic and physical regions of Rps2b defined by the two most closely linked markers are marked with solid dark bars. The right bar (c) provides the physical location of the Rps2b initial mapping region on Chromosome 16 based on BSA analysis. The Circle represents approximate position of centromere, whereas the two bars connected to centromere represent two arms of chromosome.

FIGS. 5A-5C provide the experimental results showing the expression pattern of the 3 tested Rps2b locus R-genes in the recombinants. The phenotype of first four recombinants (10675, 5643, 664, 7855) are all heterozygous resistance, while the phenotypes of the last four recombinants (2123, 5303, 3600, 131-4) are all susceptible. FIG. 5A provides the results of Rps2b R-gene2. FIG. 5B provides the results of Rps2b R-gene3. FIG. 5C provides the results of Rps2b R-gene4.

FIGS. 6A and 6B provide graphs of experimental results of a complementation test to P. sojae Race 1 (FIG. 6A) and Race 25 (FIG. 6B) in transgenic soybean plants expressing the Rps2b gene, soybean variety F₅ RIL (positive control line), or soybean Williams (negative control).

FIG. 6C provides a graph of experimental results providing the relative expression level of Rps2b in the transgenic soybean plants expressing the Rps2b gene, the soybean variety F₅ RIL (positive control line), and the soybean Williams (negative control).

FIG. 7 provides experimental results depicting the initial mapping of Rps15 from the bulked segregant analysis (BSA). Circles represent the genotypes of the resistant bulk at the SNP sites between the two parental lines, while triangles are the genotypes of the susceptible bulk at the same set of SNP sites. The x-axis shows the physical positions of these SNP sites along chromosome 16 and on the y-axis, the T1 position represents homozygous genotype detected in the resistant bulk, and the T2 position indicates the heterozygous genotypes that were detected in susceptible or/and resistant bulks. Dotted vertical line a and b define two boundaries of Rps15 region from initial BSA mapping.

FIG. 8 provides the genetic and physical map of the Rps15 region. The left bar (a) provides a genetic map of the Rps15 region according to linkage analysis. Molecular markers (SSR/InDels) are listed on the left side and the genetic distance (centimorgan, cM) between adjacent markers are shown on the right side of the map. The center bar (b) provides the physical positions of molecular markers on chromosome 16 based on soybean reference genome (Wms82 v2.1). Both genetic and physical regions of Rps15 defined by two most closely linked markers are marked with solid dark bars. Fine mapping region of RpsUN2 is within the region defined by BARCSOY 16_1288 and BARCSOY_16_1297 (Li et al. 2016). The right bar (c) provides the physical location of the Rps15 initial mapping region on Chromosome 16 based on BSA analysis. Circle represents approximate position of centromere, whereas two bars connected to centromere represent two arms of chromosome.

FIG. 9 provides experimental results depicting the initial mapping of Rp1f from bulked segregant analysis (BSA). Circles represent the genotypes of the resistant bulk at the SNP sites between the two parental lines, while triangles are genotypes of the susceptible bulk at the same set of SNP sites. The x-axis shows the physical positions of these SNP sites along chromosome 3 and on the y-axis, the T1 position represents homozygous genotype detected in the susceptible bulk, and the T2 position indicates the heterozygous genotypes that were detected in susceptible or/and resistant bulks. Dotted vertical line a and b define two boundaries of Rps14 region from initial BSA mapping.

FIG. 10 provides the genetic and physical map of the Rps14 region. The left bar (a) provides the genetic map of the Rps14 region according to linkage analysis. SSR markers are listed on the left side and the genetic distance (centimorgan, cM) between adjacent markers are shown on the right side of the map. The middle bar (b) provides the physical positions of molecular markers on chromosome 3 based on soybean reference genome (Wms82 v2.1). Both genetic and physical regions of Rps14 defined by two most closely linked markers are marked with solid dark bars. The right bar (c) provides the physical location of the Rps14 initial mapping region on Chromosome 3 based on BSA analysis. The circle represents approximate position of centromere, whereas the two bars connected to centromere represent the two arms of the chromosome.

The sequence descriptions summarize the Sequence listing attached hereto, which is hereby incorporated by reference and as indicated below in Table 1. The Sequence Listing contains one letter codes for nucleotide sequence characters and the single and three letter codes for amino acids as defined in the IUPAC-IUB standards described in Nucleic Acids Research 13:3021-3030 (1985) and in the Biochemical Journal 219 (2): 345-373 (1984).

TABLE 1
Sequence Listing Description

| Polynucleotide | Polypeptide | |
|---|---|---|
| SEQ ID | SEQ ID | |
| NO: | NO: | Name |
| 1 | 2 | Rps11 |
| 3, 5 | 4 | Rps2b |
| 6, 8 | 7 | Rps2b-Rgene2 |
| 9, 11 | 10 | Rps2b-Rgene4 |
| 12 | | Rps11 forward primer |
| 13 | | Rps11 reverse primer |
| 14 | | Rps2b forward primer |
| 15 | | Rps2b reverse primer |
| 16 | | Rps2b-Rgene2 forward primer |
| 17 | | Rps2b-Rgene2 reverse primer |
| 18 | | Rps2b-Rgene4 forward primer |
| 19 | | Rps2b-Rgene4 reverse primer |
| 20 | | Satt431_F |
| 21 | | Satt431_R |
| 22 | | BARCSOYSSR_16_1277_F |
| 23 | | BARCSOYSSR_16_1277_R |
| 24 | | BARCSOYSSR_16_1281_F |
| 25 | | BARCSOYSSR_16_1281_R |
| 26 | | BARCSOYSSR_16_1288_F |
| 27 | | BARCSOYSSR_16_1288_R |
| 28 | | BARCSOYSSR_16_1294_F |
| 29 | | BARCSOYSSR_16_1294_R |
| 30 | | BARCSOYSSR_16_1297_F |
| 31 | | BARCSOYSSR_16_1297_R |
| 32 | | BARCSOYSSR_16_1312_F |
| 33 | | BARCSOYSSR_16_1312_R |
| 34 | | BARCSOYSSR_16_1322_F |
| 35 | | BARCSOYSSR_16_1322_R |
| 36 | | MappingMarker-KASP-Gm16_36635129_A_G_Susceptible |
| 37 | | MappingMarker-KASP-Gm16_36635129_A_G_Resistant |
| 38 | | MappingMarker-KASP-Gm16_36641187_T_A_Susceptible |
| 39 | | MappingMarker-KASP-Gm16_36635129_A_G_Resistant |
| 40 | | MappingMarker-KASP-Gm16_36687038_T_C_Susceptible |
| 41 | | MappingMarker-KASP-Gm16_36687038_T_C_Resistant |
| 42 | | MappingMarker-KASP-Gm16_36742320_G_A_Susceptible |
| 43 | | MappingMarker-KASP-Gm16_36742320_G_A_Resistant |
| 44 | | MappingMarker-KASP-Gm16_36745870_T_C_Susceptible |
| 45 | | MappingMarker-KASP-Gm16_36745870_T_C_Resistant |
| 46 | | MappingMarker-KASP-Gm16_36789567_C_T_Susceptible |
| 47 | | MappingMarker-KASP-Gm16_36789567_C_T_Resistant |
| 48 | | MappingMarker-KASP-Gm16_36805209_C_G_Susceiptble |
| 49 | | MappingMarker-KASP-Gm16_36805209_C_G_Resistant |
| 50 | | MsppingMarker-KASP-Gm16_36818299_T_A_Susceptible |
| 51 | | MappingMarker-KASP-Gm16_36818299_T_A_Resistant |
| 52 | | MappingMarker-KASP-Gm16_36825046_C_T_Susceptible |
| 53 | | MappingMarker-KASP-Gm16_36825046_C_T_Resistant |
| 54 | | MappingMarker-KASP-Gm16_36840817_A_G_Susceptible |
| 55 | | MappingMarker-KASP-Gm16_36840817_A_G_Resistant |
| 56 | | MappingMarker-KASP-Gm16_36844181_C_T_Susceptible |
| 57 | | MappingMarker-KASP-Gm16_36844181_C_T_Resistant |
| 58 | | MappingMarker-KASP-Gm16_36849203_G_A_Susceptible |
| 59 | | MappingMarker-KASP-Gm16_36849203_G_A_Resistant |
| 60 | | MappingMarker-KASP-Gm16_36854790_A_T_Susceptible |
| 61 | | MappingMarker-KASP-Gm16_36854790_A_T_Resistant |
| 62 | | MappingMarker-KASP-Gm16_36870179_G_C_Susceptible |
| 63 | | MappingMarker-KASP-Gm16_36870179_G_C_Resistant |
| 64 | | MappingMarker-KASP-Gm16_36889339_T_G_Susceptible |
| 65 | | MappingMarker-KASP-Gm16_36889339_T_G_Resistant |
| 66 | | MappingMarker-SSR-07-286F |
| 67 | | MappingMarker-SSR-07-286R |
| 68 | | MappingMarker-SSR-07-295F |
| 69 | | MappingMarker-SSR-07-295R |
| 70 | | MappingMarker-InDel-626F |
| 71 | | MappingMarker-InDel-626R |
| 72 | | MappingMarker-InDel-5UTR-43F |
| 73 | | MappingMarker-InDel-5UTR-43R |
| 74 | | MappingMarker-SSR-07-300F |
| 75 | | MappingMarker-SSR-07-300R |
| 76 | | MappingMarker-176kb-F |
| 77 | | MappingMarker-176kb-R |
| 78 | | MappingMarker-InDel-327kb-F |
| 79 | | MappingMarker-InDel-327kb-R |
| 80 | | MappingMarker-InDel-5.922-F |
| 81 | | MappingMarker-InDel-5.922-R |
| 82 | | MappingMarker-InDel-6.036-F |
| 83 | | MappingMarker-InDel-6.036-R |
| 84 | | MappingMarker-SSR-07-320F |
| 85 | | MappingMarker-SSR-07-320R |
| 86 | | InDel3155-F |
| 87 | | InDel3155-R |
| 88 | | InDe13255-F |
| 89 | | InDe13255-R |
| 90 | | InDel3304-F |
| 91 | | InDe13304-R |
| 92 | | InDe13437-F |
| 93 | | InDe13437-R |
| 94 | | InDel3575-F |
| 95 | | InDel3575-R |
| 96 | | InDel3568-F |
| 97 | | InDel3568-R |
| 98 | | InDel3668-F |
| 99 | | InDe13668-R |
| 100 | | InDe13678-F |
| 101 | | InDel3678-R |
| 102 | | InDel3701-F |
| 103 | | InDe13701-R |
| 104 | | Satt631-F |
| 105 | | Satt631-R |
| 106 | | BARCSOYSSR_03_0209-F |
| 107 | | BARCSOYSSR_03_0209-R |
| 108 | | BARCSOYSSR_03_0219-F |
| 109 | | BARCSOYSSR_03_0219-R |
| 110 | | BARCSOYSSR_03_0226-F |
| 111 | | BARCSOYSSR_03_0226-R |
| 112 | | BARCSOYSSR_03_0229-F |
| 113 | | BARCSOYSSR_03_0229-R |
| 114 | | BARCSOYSSR_03_0266-F |
| 115 | | BARCSOYSSR_03_0266-R |
| 116 | | InDe13971-F |
| 117 | | InDel3971-R |
| 118 | | InDe14033-F |
| 119 | | InDe14033-R |
| 120 | | InDel4263-F |
| 121 | | InDel4263-R |
| 122 | | InDel4330-F |
| 123 | | InDe14330-R |

### DETAILED DESCRIPTION

### I. Compositions

A. Rps Polynucleotides and Polypeptides One aspect of the disclosure provides a polynucleotide encoding a resistant to Phytophthora sojae (Rps) polypeptide comprising an amino acid sequence that is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2, 4, 7 or 10.

As used herein “encoding,” “encoded,” or the like, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal and fungal mitochondria, the bacterium Mycoplasma capricolum (Yamao, et al., (1985) Proc. Natl. Acad. Sci. USA 82:2306-9) or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledonous plants or dicotyledonous plants as these preferences have been shown to differ (Murray, et al., (1989) Nucleic Acids Res. 17:477-98 and herein incorporated by reference).

The terms “polypeptide,” “peptide” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers.

As used herein “percent (%) sequence identity” with respect to a reference sequence (subject) is determined as the percentage of amino acid residues or nucleotides in a candidate sequence (query) that are identical with the respective amino acid residues or nucleotides in the reference sequence, after aligning the sequences and introducing gaps, if necessary, to achieve the maximum percent sequence identity, and not considering any amino acid conservative substitutions as part of the sequence identity. Alignment for purposes of determining percent sequence identity can be achieved in various ways that are within the skill in the art, for instance, using publicly available computer software such as BLAST, BLAST-2. Those skilled in the art can determine appropriate parameters for aligning sequences, including any algorithms needed to achieve maximal alignment over the full length of the sequences being compared. The percent identity between the two sequences is a function of the number of identical positions shared by the sequences (e.g., percent identity of query sequence=number of identical positions between query and subject sequences/total number of positions of query sequence×100).

Unless otherwise stated, sequence identity/similarity values provided herein refer to the value obtained using the BLAST 2.0 suite of programs using default parameters (Altschul, et al., (1997) Nucleic Acids Res. 25:3389-402).

### B. Recombinant DNA Construct

Also provided is a recombinant DNA construct comprising any of the Rps polynucleotides described herein. In certain embodiments, the recombinant DNA construct further comprises at least one regulatory element. In certain embodiments, the at least one regulatory element of the recombinant DNA construct comprises a promoter. In certain embodiments, the promoter is the native Rps polynucleotide promoter sequence. In certain embodiments, the promoter is heterologous to the Rps polynucleotide sequence.

As used herein, a “recombinant DNA construct” comprises two or more operably linked DNA segments, preferably DNA segments that are not operably linked in nature (i.e., heterologous). Non-limiting examples of recombinant DNA constructs include a polynucleotide of interest operably linked to regulatory elements, which aid in the expression, autologous replication, and/or genomic insertion of the sequence of interest. Such regulatory elements include, for example, promoters, expression modulating elements (EMEs), termination sequences, enhancers, etc., or any component of an expression cassette; a plasmid, cosmid, virus, autonomously replicating sequence, phage, or linear or circular single-stranded or double-stranded DNA or RNA nucleotide sequence; and/or sequences that encode heterologous polypeptides.

The Rps polynucleotides described herein can be provided in expression cassettes for expression in a plant of interest or any organism of interest. The cassette can include 5′ and 3′ regulatory sequences operably linked to a Rps polynucleotide. “Operably linked” is intended to mean a functional linkage between two or more elements. For, example, an operable linkage between a polynucleotide of interest and a regulatory sequence (e.g., a promoter) is a functional link that allows for expression of the polynucleotide of interest. Operably linked elements may be contiguous or non-contiguous. When used to refer to the joining of two protein coding regions, operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the Rps polynucleotide to be under the transcriptional regulation of the regulatory regions. The expression cassette may additionally contain selectable marker genes.

The expression cassette can include in the 5′-3′ direction of transcription, a transcriptional and translational initiation region (e.g., a promoter), a Rps polynucleotide, and a transcriptional and translational termination region (e.g., termination region) functional in plants. The regulatory regions (e.g., promoters, transcriptional regulatory regions, and translational termination regions) and/or the Rps polynucleotide may be native/analogous to the host cell or to each other. Alternatively, the regulatory regions and/or the Rps polynucleotide may be heterologous to the host cell or to each other.

As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide that is from a species different from the species from which the polynucleotide was derived, or, if from the same/analogous species, one or both are substantially modified from their original form and/or genomic locus, or the promoter is not the native promoter for the operably linked polynucleotide.

The termination region may be native with the transcriptional initiation region, with the plant host, or may be derived from another source (i.e., foreign or heterologous) than the promoter, the Rps polynucleotide, the plant host, or any combination thereof.

The expression cassette may additionally contain a 5′ leader sequences. Such leader sequences can act to enhance translation. Translation leaders are known in the art and include viral translational leader sequences.

In preparing the expression cassette, the various DNA fragments may be manipulated, to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

As used herein “promoter” refers to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to, those that are obtained from plants, plant viruses and bacteria which comprise genes expressed in plant cells such Agrobacterium or Rhizobium. Certain types of promoters preferentially initiate transcription in certain tissues, such as leaves, roots, seeds, fibres, xylem vessels, tracheids or sclerenchyma. Such promoters are referred to as “tissue preferred.” A “cell type” specific promoter primarily drives expression in certain cell types in one or more organs, for example, vascular cells in roots or leaves. An “inducible” or “regulatable” promoter is a promoter, which is under environmental control. Examples of environmental conditions that may affect transcription by inducible promoters include anaerobic conditions or the presence of light. Another type of promoter is a developmentally regulated promoter, for example, a promoter that drives expression during pollen development. Tissue preferred, cell type specific, developmentally regulated and inducible promoters constitute the class of “non-constitutive” promoters. A “constitutive” promoter is a promoter, which is active under most environmental conditions. Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026); GOS2 (U.S. Pat. No. 6,504,083), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Also contemplated are synthetic promoters which include a combination of one or more heterologous regulatory elements.

The promoter of the recombinant DNA constructs described herein can be any type or class of promoter known in the art, such that any one of a number of promoters can be used to express the various Rps polynucleotide sequences disclosed herein, including the native promoter of the polynucleotide sequence of interest. The promoters for use in the recombinant DNA constructs of the invention can be selected based on the desired outcome.

In certain embodiments, the recombinant DNA construct, described herein, is expressed in a plant or seed. In certain embodiments, the plant or seed is a soybean plant or soybean seed. The polynucleotides or recombinant DNA constructs disclosed herein may be used for transformation of any plant species.

### C. Plants and Plant Cells

Provided are plants, plant cells, plant parts, seeds, and grain comprising at least one of the Rps polynucleotide sequences or recombinant DNA constructs, described herein, so that the plants, plant cells, plant parts, seeds, and/or grain express any of the Rps polypeptides described herein. In certain embodiments, the plants, plant cells, plant parts, seeds, and/or grain have stably incorporated at least one Rps polynucleotide described herein into its genome. In certain embodiments, the plants, plant cells, plant parts, seeds and grain are soybean plants, plant cells, plant parts, seeds and grain. In certain embodiments, the plants, plant cells, plant parts, seeds, and/or grain can comprise multiple Rps polynucleotides (i.e., at least 1, 2, 3, 4, 5, 6 or more).

Also provided are plants, plant cells, plant parts, seeds, and grain comprising a targeted genetic modification increasing expression of a polynucleotide encoding a polypeptide comprising an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 2, 4, 7 or 10 as compared to a control plant not comprising the targeted genetic modification. In certain embodiments, the plants, plant cells, plant parts, seeds and grain are soybean plants, plant cells, plant parts, seeds and grain. In certain embodiments, the plant (e.g., soybean plant) comprising the targeted genetic modification has improved resistance to Phytophthora infection as compared to the control plant. In certain embodiments, the targeted genetic modification comprises the insertion of a polynucleotide sequence comprising and one of SEQ ID NOs: 1, 3, 6 and 9 into the genome of the plant.

“Phytophthora”, “Phtophthora sojae”, and “P. sojae” are used interchangeably herein and refer to the soil-born oomycete pathogen that is the causative agent for Phytophthora root and stem rot.

As used herein “increasing expression” “increased expression” or the like refers to any detectable increase in the level of the polynucleotide or encoded polypeptide as compared to a control plant (e.g., non-modified plant). The level of expression can be measure using routine methods known in the art such as PCR, Western blotting, mass spectrometry, and ELISA.

As used herein, a “targeted” genetic modification or “targeted” DNA modification, refers to the direct manipulation of an organism's genes. The targeted modification may be introduced using any technique known in the art, such as, for example, plant breeding, genome editing, or single locus conversion.

The DNA modification of the genomic locus may be done using any genome modification technique known in the art or described herein. In certain embodiments the targeted DNA modification is through a genome modification technique selected from the group consisting of a polynucleotide-guided endonuclease, CRISPR-Cas endonucleases, base editing deaminases, zinc finger nuclease, a transcription activator-like effector nuclease (TALEN), engineered site-specific meganuclease, or Argonaute.

In certain embodiments, the genome modification may be facilitated through the induction of a double-stranded break (DSB) or single-strand break, in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), guided cpf1 endonuclease systems, and the like. In some embodiments, the introduction of a DSB can be combined with the introduction of a polynucleotide modification template.

As used herein, the term “plant” includes plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the disclosure, provided that these parts comprise the introduced polynucleotides.

Also provided are plants, plant cells, plant parts, seeds, and grain comprising a polynucleotide encoding a Rps polypeptide comprising an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 2, 4, 7 or 10 operably linked to a regulatory element. In certain embodiments, the regulatory element is the native Rps promoter sequence. In certain embodiments, the regulatory element is a heterologous regulatory element, such as, for example, a heterologous promoter.

In certain embodiments, the plants described herein (e.g., plants comprising an Rps polynucleotide described herein) have a yield of soybean seeds by weight at 13% moisture that is at least 80%, 81%, 82%, 83%, 84%, 85%, 86%, 87%, 88%, 89%, 90%, 91%, 92%, 93%, 94%, 95%, 96%, 97%, 98% 99%, 100%, 101%, 102%, 103%, 104%, 105%, 106%, 107%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134% or 135% and less than 250%, 240%, 203%, 220%, 210%, 200%, 195%, 190%, 185%, 180%, 175%, 170%, 165%, 160%, 155%, 150%, 145% or 140% of the yield of seeds by weight of soybean variety 93B83 (U.S. Pat. No. 5,792,909), when grown under the same environmental conditions. Representative seed of soybean variety 93B83 were deposited under ATCC Accession No. 209766 on Apr. 10, 1998. As used herein, “under the same environmental conditions” means the plants are grown in proximity in the field or a greenhouse under non-stress conditions suitable for growth of a soybean plant to maturity, with the plants being exposed to the same environment and seeds harvested from each plant at maturity growth stage R8.

Applicant has made a deposit of at least 2500 seeds of Soybean Variety 93B83 with the American Type Culture Collection (ATCC), 10801 University Boulevard, Manassas, Va. 20110 USA, as ATCC Deposit No. 209766. The seeds were deposited with the ATCC on Apr. 10, 1998 have been accepted under the Budapest Treaty. This deposit of the Soybean Variety 93B83 will be maintained in the ATCC depository, which is a public depository, for a period of 30 years, or 5 years after the most recent request, or for the effective life of the patent, whichever is longer, and will be replaced if it becomes nonviable during that period. Additionally, Applicant has satisfied all the requirements of 37 C.F.R. §§ 1.801-1.809. Upon allowance of any claims in the application, the Applicant(s) will maintain and will make this deposit available to the public pursuant to the Budapest Treaty.

As used herein, “yield” refers to the amount of agricultural production harvested per unit of land and may include reference to bushels per acre or kilograms per hectare of a crop at harvest, as adjusted for grain moisture. Grain moisture is measured in the grain at harvest. The adjusted test weight of grain is determined to be the weight in pounds per bushel or kilogram, adjusted for grain moisture level at harvest.

### II. Methods

### A. Methods for Increasing Phytophthora Resistance

Provided are methods for generating a Phytophthora resistant soybean plant or increasing resistance to Phytophthora in a soybean plant comprising expressing in a soybean plant a polynucleotide encoding a Rps polypeptide comprising an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 2, 4, 7 or 10.

“Resistance” and “improved resistance” are used interchangeably herein and refer to any type of increase in resistance or tolerance to, or any type of decrease in susceptibility. A “resistant plant” or “resistant plant variety” need not possess absolute or complete resistance. Instead, a “resistant plant,” “resistant plant variety,” or a plant or plant variety with “improved tolerance” will have a level of resistance or tolerance which is higher than that of a comparable susceptible plant or variety. A person of ordinary skill in the art can readily identify plants having resistance or improved resistance to Phytophthora using methods known in the art.

The plants of the compositions and methods described herein can have resistance or improved resistance to any race or isolate of Phytophthora sojae known in the art (e.g., any one of races 1 to 55). In certain embodiments, the plants are resistant or have improved resistance to at least 1, 2, 3, 4, 5, 6, 7, 8, 9, 10, 11, 12, 13, 14, 15, 16, 17, 18, 19, 20 races and/or isolates of Phytophthora sojae. In certain embodiments, the plants are resistant or have improved resistance to at least one, at least two, at least three, at least four, at least five, at least six, at least seven, at least eight, at least nine, or at least 10 races and/or isolates of Phytophthora sojae selected from the group consisting of Race1, Race 2, Race3, Race4, Race5, Race6, Race7, Race8, Race9 Race13, Race17, Race25, Race31, OH001, OHC2S1, OH003, ISA19A-1, ISA71D-1, MIN12001.01.05, MIN12002.01.05, MIN12002.05.01, MIN12002.05.05, MIN12004.01.01, MIN12004.03.01, MIN12005.07.02. In certain embodiments, the plant has resistance to Race1, Race3, Race4, Race7, Race13, Race17, Race25, ISA19A-1, ISA71D-1, MIN12001.01.05, MIN12004.01.01, MIN12004.03.01, MIN12005.07.02. In certain embodiments, the plant has resistance to Race1, Race 2, Race3, Race4, Race5, Race6, Race7, Race8, Race9, Race13, Race17, Race25, ISA19A-1, ISA71D-1, MIN12001.01.05, MIN12004.01.01, MIN12004.03.01, MIN12005.07.02. In certain embodiments, the plant has resistance to Race1, Race3, Race4, Race7, Race13, Race17, Race25, Race31, OH001, OHC2S1, OH003, MIN12002.01.05, MIN12002.05.01, MIN12002.05.05, ISA19A-1, ISA71D-1, MIN12001.01.05, MIN12004.01.01, MIN12004.03.01, MIN12005.07.02.

In certain embodiments, the method comprises introducing in a regenerable soybean plant cell a targeted genetic modification increasing the expression of a polynucleotide encoding a polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of SEQ ID NOs: 2, 4, 7 or 10 and generating the plant wherein the plant has increased expression of the polynucleotide and increased resistance to Phytophthora infection as compared to a control plant not comprising the targeted genetic modification. In certain embodiments, the method comprises introducing in a regenerable soybean plant cell a targeted genetic modification that inserts an exogenous polynucleotide into the plant cell genome, wherein the exogenous polynucleotide encodes a polypeptide comprising an amino acid sequence having at least 95%, 96%, 97%, 98%, or 99% sequence identity to SEQ ID NOs: 2, or 4 and generating the plant from the transformed plant cell, wherein the generated plant expresses the polynucleotide and has increased resistance to Phytophthora infection as compared to a control plant not comprising the exogenous polynucleotide.

In certain embodiments, the method comprises introducing in a regenerable soybean plant cell isolated from a soybean plant susceptible to at least one race of Phytophthora a polynucleotide encoding a polypeptide comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of SEQ ID NOs: 2, 4, 7 or 10, and generating the plant wherein the plant expresses the polynucleotide and is resistant to the at least one race of Phytophthora as compared to a control plant not expressing the polynucleotide. In certain embodiments, the polynucleotide is introduced in the regenerable soybean plant cell using a targeted genetic modification. In certain embodiments, the polynucleotide is introduced in the regenerable soybean plant cell by a recombinant DNA construct comprising the polynucleotide operably linked to a regulatory element, optionally wherein the regulatory element is a heterologous plant promoter.

In certain embodiments, the method comprises providing a guide RNA, at least one polynucleotide modification template, and at least one Cas endonuclease to a soybean plant cell, wherein the at least one Cas endonuclease introduces a double strand break at a locus in the soybean plant cell, and wherein the polynucleotide modification template introduces and/or inserts a polynucleotide encoding a polypeptide comprising an amino acid sequence that is at least 80% identical to any one of SEQ ID NOs: 2, 4, 7 or 10, obtaining a soybean plant from the soybean plant cell, and generating a progeny plant that comprises the polynucleotide and has increased resistance to Phytophthora as compared to a control pant not comprising the targeted genetic modification. In certain embodiments, the locus in the soybean plant cell is an endogenous Rps gene or locus. In certain embodiments, the Cas endonuclease is Cas9.

In certain embodiments of the methods described herein, the regenerable plant cell is derived from a soybean plant that is susceptible to at least on race of Phytophthora.

In certain embodiments of the methods described herein, the targeted genetic modification is introduced using a genome modification technique selected from the group consisting of polynucleotide-guided endonuclease, CRISPR-Cas endonucleases, based editing deaminases, a zinc finger nuclease, a transcription activator-like effector nuclease (TALEN), engineered site-specific meganucleases, or Argonaute.

Various methods can be used to introduce a genetic modification at a genomic locus that encodes a Rps polypeptide into the plant, plant part, plant cell, seed, and/or grain. In certain embodiments the targeted DNA modification is through a genome modification technique selected from the group consisting of a polynucleotide-guided endonuclease, CRISPR-Cas endonucleases, base editing deaminases, zinc finger nuclease, a transcription activator-like effector nuclease (TALEN), engineered site-specific meganuclease, or Argonaute.

In certain embodiments, the genome modification may be facilitated through the induction of a double-stranded break (DSB) or single-strand break, in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), guided cpf1 endonuclease systems, and the like. In some embodiments, the introduction of a DSB can be combined with the introduction of a polynucleotide modification template.

The process for editing a genomic sequence combining DSB and modification templates generally comprises: providing to a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB.

In one embodiment the DSB-inducing agent is sequence specific endonuclease. The endonuclease can be provided to a cell by any method known in the art, for example, but not limited to, transient introduction methods, transfection, microinjection, and/or topical application or indirectly via recombination constructs. The endonuclease can be provided as a protein or as a guided polynucleotide complex directly to a cell or indirectly via recombination constructs. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433 published May 12, 2016.

TAL effector nucleases (TALEN) are a class of sequence-specific nucleases that can be used to make double-strand breaks at specific target sequences in the genome of a plant or other organism (Miller et al. (2011) Nature Biotechnology 29:143-148).

Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (patent application PCT/US12/30061, filed on Mar. 22, 2012). Meganucleases have been classified into four families based on conserved sequence motifs, the families are the LAGLIDADG, GIY-YIG, H—N—H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. The cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families.

Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a non-specific endonuclease domain, for example nuclease domain from a Type IIs endonuclease such as FokI. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3-finger domain recognized a sequence of 9 contiguous nucleotides, with a dimerization requirement of the nuclease, two sets of zinc finger triplets are used to bind an 18-nucleotide recognition sequence.

Genome editing using DSB-inducing agents, such as Cas9-gRNA complexes, has been described, for example in U.S. Patent Application US 2015-0082478 A1, WO2015/026886 A1, WO2016007347, and WO201625131 all of which are incorporated by reference herein.

In certain embodiments the genetic modification is introduced without introducing a double strand break using base editing technology, see e.g., Gaudelli et al., (2017) Programmable base editing of A*T to G*C in genomic DNA without DNA cleavage. Nature 551 (7681): 464-471; Komor et al., (2016) Programmable editing of a target base in genomic DNA without double-stranded DNA cleavage, Nature 533 (7603): 420-4.

In certain embodiments, base editing comprises (i) a catalytically impaired CRISPR-Cas9 mutant that is mutated such that one of their nuclease domains cannot make DSBs; (ii) a single-strand-specific cytidine/adenine deaminase that converts C to U or A to G within an appropriate nucleotide window in the single-stranded DNA bubble created by Cas9; (iii) a uracil glycosylase inhibitor (UGI) that impedes uracil excision and downstream processes that decrease base editing efficiency and product purity; or (iv) nickase activity to cleave the non-edited DNA strand, followed by cellular DNA repair processes to replace the G-containing DNA strand.

In certain embodiments, the targeted genetic modification is selected from the group consisting of an insertion, deletion, single nucleotide polymorphism (SNP), and a polynucleotide modification. In certain embodiments, the targeted genetic modification is present in (a) the coding region; (b) a non-coding region; (c) a regulatory sequence; (d) an untranslated region; or (e) any combination of (a)-(d) of the genomic locus that encodes the RPS polypeptide.

In certain embodiments the DNA modification increasing the level and or activity of the RPS polypeptide is an insertion of one or more nucleotides, preferably contiguous, in the genomic locus. For example, the insertion of an expression modulating element (EME), such as an EME described in PCT/US2018/025446 (WO2018183878), in operable linkage with the RPS gene. In certain embodiments, the targeted DNA modification may be the replacement of the endogenous RPS promoter with another promoter known in the art to have higher expression. In certain embodiments, the targeted DNA modification may be the insertion of a promoter known in the art to have higher expression into the 5′UTR so that expression of the endogenous RPS polypeptide is controlled by the inserted promoter. In certain embodiments, the DNA modification is a modification to optimize Kozak context to increase expression. In certain embodiments, the DNA modification is a polynucleotide modification or SNP at a site that regulates the stability of the expressed protein.

In certain embodiments, the method comprises expressing in a regenerable soybean plant cell any of the recombinant DNA constructs described herein and generating the plant wherein the plant has increased expression of the polynucleotide and increased resistance to Phytophthora infection as compared to a control plant not comprising the recombinant DNA construct.

Various methods can be used to introduce the RPS sequences (e.g., modified RPS sequence or recombinant DNA comprising the modified RPS sequence) into a plant, plant part, plant cell, seed, and/or grain. “Introducing” is intended to mean presenting to the plant, plant cell, seed, and/or grain the inventive polynucleotide or resulting polypeptide in such a manner that the sequence gains access to the interior of a cell of the plant. The methods of the disclosure do not depend on a particular method for introducing a sequence into a plant, plant cell, seed, and/or grain, only that the polynucleotide or polypeptide gains access to the interior of at least one cell of the plant.

“Stable transformation” is intended to mean that the polynucleotide introduced into a plant integrates into the genome of the plant of interest and is capable of being inherited by the progeny thereof. “Transient transformation” is intended to mean that a polynucleotide is introduced into the plant of interest and does not integrate into the genome of the plant or organism or a polypeptide is introduced into a plant or organism.

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606), Agrobacterium-mediated transformation (U.S. Pat. Nos. 5,563,055 and 5,981,840), Ochrobacterium-mediated transformation (U.S. Patent Application Publication 2018/0216123 and WO20/092494) direct gene transfer (Paszkowski et al. (1984) EMBO J. 3:2717-2722), and ballistic particle acceleration (see, for example, U.S. Pat. Nos. 4,945,050; U.S. Pat. Nos. 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In specific embodiments, the RPS sequences can be provided to a plant using a variety of transient transformation methods. Such transient transformation methods include, but are not limited to, the introduction of the RPS protein directly into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91:2176-2180 and Hush et al. (1994) The Journal of Cell Science 107:775-784, all of which are herein incorporated by reference.

In other embodiments, the inventive polynucleotides disclosed herein may be introduced into plants by contacting plants with a virus or viral nucleic acids. Generally, such methods involve incorporating a nucleotide construct of the disclosure within a DNA or RNA molecule. It is recognized that the inventive polynucleotide sequence may be initially synthesized as part of a viral polyprotein, which later may be processed by proteolysis in vivo or in vitro to produce the desired recombinant protein. Further, it is recognized that promoters disclosed herein also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the polynucleotide disclosed herein can be contained in transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided, and the transfer cassette is integrated at the target site. The polynucleotide of interest is thereby integrated at a specific chromosomal position in the plant genome. Other methods to target polynucleotides are set forth in WO 2009/114321 (herein incorporated by reference), which describes “custom” meganucleases produced to modify plant genomes, in particular the genome of maize. See, also, Gao et al. (2010) Plant Journal 1:176-187.

One of skill will recognize that after the expression cassette containing the inventive polynucleotide is stably incorporated in transgenic plants and confirmed to be operable, it can be introduced into other plants by sexual crossing. Any of a number of standard breeding techniques can be used, depending upon the species to be crossed.

Parts obtained from the regenerated plants described herein, such as flowers, seeds, leaves, branches, fruit, and the like are included, provided that these parts comprise cells comprising the inventive polynucleotide. Progeny and variants, and mutants of the regenerated plants are also included, provided that these parts comprise the introduced nucleic acid sequences.

In one embodiment, a homozygous transgenic plant can be obtained by sexually mating (selfing) a heterozygous transgenic plant that contains a single added heterologous nucleic acid, germinating some of the seed produced and analyzing the resulting plants produced for altered cell division relative to a control plant (i.e., native, non-transgenic). Back-crossing to a parental plant and out-crossing with a non-transgenic plant are also contemplated.

### B. Breeding Method for Increasing Phytophthora Resistance

Further provided are methods of producing plants having increased Phytophthora resistance comprising crossing a plant comprising a targeted genetic modification increasing expression of a polynucleotide encoding a polypeptide comprising an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 2, 4, 7 or 10 as compared to a control plant not comprising the targeted genetic modification with a second plant line and harvesting the seed produced thereby. In certain embodiments, the seed produced thereby comprises the polynucleotide. In certain embodiments, the second plant is susceptible to Phytophthora and the harvested seed produces a plant that is resistant and/or has increased resistance to Phytophthora.

In certain embodiments, the method further comprises growing the seed to produce a second-generation progeny plant that comprises the polypeptide and backcrossing the second-generation progeny plant to the second plant to produce a backcross progeny plant that comprises the polypeptide and produces backcrossed seed with increased Phytophthora resistance.

Also provided are methods of producing plants having increased Phytophthora resistance comprising crossing a plant comprising increased expression of a polynucleotide encoding a polypeptide comprising an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to any one of SEQ ID NOs: 2, 4, 7 or 10 as compared to a control plant not comprising the polynucleotide with a second plant line and harvesting the seed produced thereby. In certain embodiments, the plant comprises a recombinant DNA construct described herein. In certain embodiments, the seed produced thereby comprises the polynucleotide. In certain embodiments, the second plant is susceptible to Phytophthora and the harvested seed produces a plant that is resistant and/or has increased resistance to Phytophthora.

In certain embodiments, the method further comprises growing the seed to produce a second-generation progeny plant that comprises the polypeptide and backcrossing the second-generation progeny plant to the second plant to produce a backcross progeny plant that comprises the polypeptide and produces backcrossed seed with increased Phytophthora resistance.

### C. Methods to Detect a Phytophthora Resistant Plant

Also provided herein are methods for detecting a Phytophthora resistant plant comprising identifying plants comprising a nucleic acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2, 4, 7 or 10. In certain embodiments, the method comprises detecting the nucleic acid sequence using primers and probes that selectively detect a polynucleotide comprising a nucleic acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 2, 4, 7 or 10.

Also provided herein are methods for detecting a Phytophthora resistant plant comprising detecting a molecular marker linked to or associated with Rps11, Rps2b, Rps15, or Rps14.

In certain embodiments, the method comprises detecting a marker linked to or associated with Rps11. In certain embodiments, the method comprises detecting in germplasm of a soybean plant at least one allele of a marker locus within a chromosomal interval comprising and flanked by SSR-07-286 (SEQ ID NOs: 66 and 67) and SSR-07-320 (SEQ ID NOs: 84 and 85). In certain embodiments, the method comprises detecting in germplasm of a soybean plant at least one allele of a marker locus within a chromosomal interval comprising and flanked by maker_176 kb (SEQ ID NOs: 76 and 77) and InDel_327 kb (SEQ ID NOs: 78 and 79). In certain embodiments, the method comprises detecting a marker selected from the group consisting of SSR-07-286, SSR-07-295 (SEQ ID NOs: 68 and 69), InDel-626 (SEQ ID NOs: 70 and 71), InDel-5UTR-43 (SEQ ID NOs: 72 and 73), SSR-07-300 (SEQ ID NOs: 74 and 75), 176 kb (SEQ ID NOs: 76 and 77), InDel-327 kb (SEQ ID NOs: 78 and 79), InDel-5.922 (SEQ ID NOs: 80 and 81), InDel-6.036 (SEQ ID NOs: 82 and 83), and SSR-07-320 (SEQ ID NOs: 84 and 85).

In certain embodiments, the method comprises detecting a marker linked to or associated with Rps2b. In certain embodiments, the method comprises detecting in germplasm of a soybean plant at least one allele of a marker locus within a chromosomal interval comprising and flanked by Satt431 (SEQ ID NOs: 20 and 21) and BARCSOY_16_1322 (SEQ ID NOs: 34 and 35). In certain embodiments, the method comprises detecting in germplasm of a soybean plant at least one allele of a marker locus within a chromosomal interval comprising and flanked by BARCSOY_16_1277 (SEQ ID NOs: 22 and 23) and BARCSOY_16_1322 (SEQ ID NOs: 34 and 35). In certain embodiments, the method comprises detecting in germplasm of a soybean plant at least one allele of a marker locus within a chromosomal interval comprising and flanked by BARCSOY_16_1288 (SEQ ID NOs: 26 and 27) and BARCSOY_16_1312 (SEQ ID NOs: 32 and 33). In certain embodiments, the method comprises detecting a marker selected from the group consisting of Satt431, BARCSOY_16_1277, BARCSOY_16_1281 (SEQ ID NOs: 24 and 25), BARCSOY_16_1288, BARCSOY_16_1294 (SEQ ID NOs: 28 and 29), BARCSOY_16_1297 (SEQ ID NOs: 30 and 31), BARCSOY_16_1312, BARCSOY_16_1322, a G at Gm16_36635129 (SEQ ID NO: 37), an A at Gm16_36641187 (SEQ ID NO: 39), a C at Gm16_36687038 (SEQ ID NO: 41), an A at Gm16_36742320 (SEQ ID NO: 43), a C at Gm16_36745870 (SEQ ID NO: 45), a T at Gm16_36789567 (SEQ ID NO: 47), a G at Gm16_36805209 (SEQ ID NO: 49), an A at Gm16_36818299 (SEQ ID NO: 51), a T at Gm16_36825046 (SEQ ID NO: 53), a G at Gm16_36840817 (SEQ ID NO: 55), a T at Gm16_36844181 (SEQ ID NO: 57), an A at Gm16_36849203 (SEQ ID NO: 59), a T at Gm16_36854790 (SEQ ID NO: 61), a C at Gm16_36870179 (SEQ ID NO: 63), and a G at Gm16_36889339 (SEQ ID NO: 65).

In certain embodiments, the method comprises detecting a marker linked to or associated with Rps15. In certain embodiments, the method comprises detecting in germplasm of a soybean plant at least one allele of a marker locus within a chromosomal interval comprising and flanked by InDel3155 and InDel3701. In certain embodiments, the method comprises detecting in germplasm of a soybean plant at least one allele of a marker locus within a chromosomal interval comprising and flanked by InDel3437 and InDel3701. In certain embodiments, the method comprises detecting in germplasm of a soybean plant at least one allele of a marker locus within a chromosomal interval comprising and flanked by Satt431 and InDel3668. In certain embodiments, the method comprises detecting a marker selected from the group consisting of InDel3155, InDel3255, InDel3304, InDel3437, InDel3575, InDel3586, Satt431, InDel3668, InDel3678, InDel3701, a T at Gm_30813568, a C at Gm16_31787658, a T at Gm16_31837545, an A at Gm16_31899513, a C at Gm16_32017661, a C at Gm16_32200441, an A at Gm16_32340079, a C at Gm16_32665742, a G at Gm16_32876100, a C at Gm16_33210540, a C at Gm16_33360539, a G at Gm16_33457667, a G at Gm16_34645180, a G at Gm16_35148803, a T at Gm16_35218386, a T at Gm16_35643452, a T at Gm16_35700223, a G at Gm16_35738081, an A at Gm16_36013043, a C at Gm16_36217195, a C at Gm16_36732450, an A at Gm16_36983033, a G at Gm16_37078478, and a G at Gm16_37209075. In certain embodiments, the marker is detected by a primer comprising a nucleotide sequence of any one of SEQ ID NOs: 20-21 and 86-103. In certain embodiments, the marker is detected by a primer pair comprising a nucleotide sequences of SEQ ID NOs: 20 and 21, SEQ ID NOs: 86 and 87, SEQ ID NOs: 88 and 89, SEQ ID NOs: 90 and 91, SEQ ID NOs: 92 and 93, SEQ ID NOs: 94 and 95, SEQ ID NOs: 96 and 97, SEQ ID NOs: 98 and 99, SEQ ID NOs: 100 and 101, and SEQ ID NOs: 102 and 103.

In certain embodiments, the method comprises detecting a marker linked to or associated with Rps14. In certain embodiments, the method comprises detecting in germplasm of a soybean plant at least one allele of a marker locus within a chromosomal interval comprising and flanked by Satt631 and BARCSOY_03_0266. In certain embodiments, the method comprises detecting in germplasm of a soybean plant at least one allele of a marker locus within a chromosomal interval comprising and flanked by BARCSOY_03_0226 and BARCSOY_03_0266. In certain embodiments, the method comprises detecting a marker selected from the group consisting of Satt631, BARCSOY_03_0209, BARCSOY_03_0219, BARCSOY_03_0226, BARCSOYSSR_03_229, BARCSOY_03_0266, InDel3971, InDe14033, InDel4263, InDel4330, a T at Gm03_829023, an A at Gm03_1671384, a G at Gm03_1718435, an A at Gm03_3087237, a G at Gm03_3907697, a C at Gm03_4487138, an A at Gm03_4509101, a C at Gm03_4665923, a C at Gm03_4782127, an A at Gm03_5165511, and a C at Gm03_5217414. In certain embodiments, the marker is detected by a primer comprising a nucleotide sequence of any one of SEQ ID NOs: 104-123. In certain embodiments, the marker is detected by a primer pair comprising a nucleotide sequences of SEQ ID NOs: 104 and 105, SEQ ID NOs: 106 and 107, SEQ ID NOs: 108 and 109, SEQ ID NOs: 110 and 111, SEQ ID NOs: 112 and 113, SEQ ID NOs: 114 and 115, SEQ ID NOs: 116 and 117, SEQ ID NOs: 118 and 119, SEQ ID NOs: 120 and 121, and SEQ ID NOs: 122 and 123.

In certain embodiments, the method further comprises crossing the Phytophthora resistant plant detected by the methods described herein with a second plant to produce progeny seed. In certain embodiments, the second plant is susceptible to Phytophthora. In certain embodiments, the second plant lacks the Rps11, Rps2b, Rps15, or Rps14 gene.

In certain embodiments, the progeny seed comprises the nucleotide sequence detected using the primers and probes. In certain embodiments, the progeny seed comprises the molecular marker linked to or associated with Rps11, Rps2b, Rps15, or Rps14. In certain embodiments, the progeny seed comprises the at least one allele of a marker locus associated with Rps2b within a chromosomal interval comprising and flanked by BARCSOY_16_1288 and BARCSOY 16_1312. In certain embodiments, the progeny seed and plant produced thereby comprises the at least one allele of a marker locus associated with Rps15 within a chromosomal interval comprising and flanked by Satt431 and InDel3668. In certain embodiments, the progeny seed comprises the at least one allele of a marker locus associated with Rps14 within a chromosomal interval comprising and flanked by BARCSOY_03_0226 and BARCSOY_03_0266. In certain embodiments, the progeny seed and plant produced thereby comprises the Rps11, Rps2b, Rps15, or Rps14 gene.

Any suitable detection method known in the art can be used to detect the polynucleotide. In some examples, the presence of the polynucleotide is directly detected in unamplified genomic DNA by performing a Southern blot on a sample of genomic DNA using probes to the marker loci. In other examples, amplification-based techniques are employed. PCR, RT-PCR, and LCR are in particularly broad use as amplification and amplification-detection methods for amplifying nucleic acids of interest, thus facilitating detection of the polynucleotide. Procedures for performing Southern blotting, amplification (PCR, LCR, or the like), and many other nucleic acid detection methods are well established and are taught, e.g., in Sambrook et al., Molecular Cloning-A Laboratory Manual (3d ed.), Vol. 1-3, Cold Spring Harbor Laboratory, Cold Spring Harbor, N.Y., 2000 (“Sambrook”); Current Protocols in Molecular Biology, F. M. Ausubel et al., eds., Current Protocols, a joint venture between Greene Publishing Associates, Inc. and John Wiley & Sons, Inc., (supplemented through 2002) (“Ausubel”)) and PCR Protocols A Guide to Methods and Applications (Innis et al. eds) Academic Press Inc. San Diego, Calif. (1990) (Innis). Additional details regarding detection of nucleic acids in plants can also be found, e.g., in Plant Molecular Biology (1993) Croy (ed.) BIOS Scientific Publishers, Inc.

“Primer” refers to an oligonucleotide (synthetic or occurring naturally), which is capable of acting as a point of initiation of nucleic acid synthesis or replication along a complementary strand when placed under conditions in which synthesis of a complementary strand is catalyzed by a polymerase. Typically, primers are oligonucleotides from 10 to 30 nucleic acids in length, but longer or shorter sequences can be employed. Primers may be provided in double-stranded form, though the single-stranded form is preferred. A primer can further contain a detectable label, for example a 5′ end label.

“Probe” refers to an oligonucleotide (synthetic or occurring naturally) that is complementary (though not necessarily fully complementary) to a polynucleotide of interest and forms a duplexed structure by hybridization with at least one strand of the polynucleotide of interest. Typically, probes are oligonucleotides from 10 to 50 nucleic acids in length, but longer or shorter sequences can be employed. A probe can further contain a detectable label. The terms “label” and “detectable label” refer to a molecule capable of detection, including, but not limited to, radioactive isotopes, fluorescers, chemiluminescers, enzymes, enzyme substrates, enzyme cofactors, enzyme inhibitors, chromophores, dyes, metal ions, metal sols, semiconductor nanocrystals, ligands (e.g., biotin, avidin, streptavidin, or haptens), and the like. A detectable label can also include a combination of a reporter and a quencher, such as are employed in FRET probes or TaqMan™ probes. The term “reporter” refers to a substance or a portion thereof which is capable of exhibiting a detectable signal, which signal can be suppressed by a quencher. The detectable signal of the reporter is, e.g., fluorescence in the detectable range. The term “quencher” refers to a substance or portion thereof which is capable of suppressing, reducing, inhibiting, etc., the detectable signal produced by the reporter. As used herein, the terms “quenching” and “fluorescence energy transfer” refer to the process whereby, when a reporter and a quencher are in close proximity, and the reporter is excited by an energy source, a substantial portion of the energy of the excited state nonradiatively transfers to the quencher where it either dissipates nonradiatively or is emitted at a different emission wavelength than that of the reporter.

Chromosomal interval” designates a contiguous linear span of genomic DNA that resides in planta on a single chromosome. The genetic elements or genes located on a single chromosomal interval are physically linked. The size of a chromosomal interval is not particularly limited. In certain embodiments, the genetic elements located within a single chromosomal interval are genetically linked, typically with a genetic recombination distance of, for example, less than or equal to 20 cM, or alternatively, less than or equal to 10 cM. That is, two genetic elements within a single chromosomal interval undergo recombination at a frequency of less than or equal to 20% or 10%.

The term “introgression” or “introgressing” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., a selected allele of a marker, a quantitative trait loci (QTL), a transgene, or the like. In any case, offspring comprising the desired allele can be repeatedly backcrossed to a line having a desired genetic background and selected for the desired allele, to result in the allele becoming fixed in a selected genetic background. For example, the chromosome 3, 7, or 16 locus described herein may be introgressed into a recurrent parent that is susceptible to Phytophthora. The recurrent parent line with the introgressed gene or locus then has increased resistance to Phytophthora.

As used herein, the term “linkage” or “linked” is used to describe the degree with which one marker locus is associated with another marker locus or some other locus (for example, a Phytophthora locus). The linkage relationship between a molecular marker and a phenotype is given as a “probability” or “adjusted probability”. Linkage can be expressed as a desired limit or range. For example, in some embodiments, any marker is linked (genetically and physically) to any other marker when the markers are separated by less than 50, 40, 30, 25, 20, or 15 map units for cM). In certain embodiments, it is advantageous to define a bracketed range of linkage, for example, between 10 and 20 cM, between 10 and 30 cM, or between 10 and 40 cM. The more closely a marker is linked to a second locus, the better an indicator for the second locus that marker becomes. Thus, “closely linked loci” such as a marker locus and a second locus display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci display a recombination frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10 (e.g., about 9%, 8%, 7%, 6%, 5%, 4%, 3%, 2%, 1%, 0.75%, 0.5%, 0.25%, or less) are also said to be “proximal to” each other. Since one cM is the distance between two markers that show a 1% recombination frequency, any marker is closely linked (genetically and physically) to any other marker that is in close proximity, e.g., at or less than 10 cM distant. Two closely linked markers on the same chromosome can be positioned 9, 8, 7, 6, 5, 4, 3, 2, 1, 0.75, 0.5 or 0.25 CM or less from each other.

“Locus” and “marker locus” are used interchangeably herein and mean a position on a chromosome where a gene and/or marker is located.

A “marker” is a nucleotide sequence or encoded product thereof (e.g., a protein) used as a point of reference. For markers to be useful at detecting recombinations, they need to detect differences, or polymorphisms, within the population being monitored. For molecular markers, this means differences at the DNA level due to polynucleotide sequence differences (e.g. SSRs, RFLPs, FLPs, SNPs). The genomic variability can be of any origin, for example, insertions, deletions, duplications, repetitive elements, point mutations, recombination events, or the presence and sequence of transposable elements. Molecular markers can be derived from genomic or expressed nucleic acids (e.g., ESTs) and can also refer to nucleic acids used as probes or primer pairs capable of amplifying sequence fragments via the use of PCR-based methods. A large number of soybean molecular markers are known in the art, and are published or available from various sources, such as the SoyBase internet resource.

Markers corresponding to genetic polymorphisms between members of a population can be detected by methods well-established in the art. These include, e.g., DNA sequencing, PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also known for the detection of expressed sequence tags (ESTs) and SSR markers derived from EST sequences and randomly amplified polymorphic DNA (RAPD).

A “marker allele”, alternatively an “allele of a marker locus”, can refer to one of a plurality of polymorphic nucleotide sequences found at a marker locus in a population that is polymorphic for the marker locus.

A “marker locus” is a specific chromosome location in the genome of a species when a specific marker can be found. A marker locus can be used to track the presence of a second linked locus, e.g., a linked locus that encodes or contributes to expression of a phenotypic trait. For example, a marker locus can be used to monitor segregation of alleles at a locus, such as a QTL or single gene, that are genetically or physically linked to the marker locus.

The term “molecular marker” may be used to refer to a genetic marker, as defined above, or an encoded product thereof (e.g., a protein) used as a point of reference when identifying a linked locus. A marker can be derived from genomic nucleotide sequences or from expressed nucleotide sequences (e.g., from a spliced RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “molecular marker probe” is a nucleic acid sequence or molecule that can be used to identify the presence of a marker locus, e.g., a nucleic acid probe that is complementary to a marker locus sequence. Alternatively, in certain embodiments, a marker probe refers to a probe of any type that is able to distinguish (i.e., genotype) the particular allele that is present at a marker locus. Nucleic acids are “complementary” when they specifically hybridize in solution, e.g., according to Watson-Crick base pairing rules. Some of the markers described herein are also referred to as hybridization markers when located on an indel region, such as the non-collinear region described herein. This is because the insertion region is, by definition, a polymorphism vis a via a plant without the insertion. Thus, the marker need only indicate whether the indel region is present or absent. Any suitable marker detection technology may be used to identify such a hybridization marker, e.g., SNP technology is used in the examples provided herein.

The following are examples of specific embodiments of some aspects of the invention. The examples are offered for illustrative purposes only and are not intended to limit the scope of the invention in any way.

In accordance with embodiment 1, a soybean plant or soybean seed is provided comprising a targeted genetic modification increasing expression of a polynucleotide encoding a polypeptide comprising an amino acid sequence having at least 80% sequence identity to any one of SEQ ID NOs: 2 or 4 as compared to a control plant not comprising the targeted genetic modification.

In accordance with embodiment 2 the soybean plant or soybean seed of embodiment 1 is provided, wherein the soybean plant or a plant grown from the soybean seed comprising the targeted genetic modification has improved resistance to Phytophthora infection as compared to the control plant.

In accordance with embodiment 3 the soybean plant or soybean seed of embodiment 1 or 2 is provided, wherein the soybean plant or a plant grown from the soybean seed comprising the targeted genetic modification has improved resistance at least one race of Phytophthora selected from the group consisting of Race1, Race 2, Race3, Race4, Race5, Race6, Race7, Race8, Race9 Race13, Race17, Race25, Race31 as compared to the control plant.

In accordance with embodiment 4 the soybean plant or soybean seed of any one of embodiments 1-3 is provided, wherein the targeted genetic modification introduces a polynucleotide sequence having at least 80% sequence identity to any one of SEQ ID NOs: 1 or 3.

In accordance with embodiment 5 a plant produced by the soybean seed of any one of embodiments 1˜4 is provided.

In accordance with embodiment 6 a method of plant breeding is provided comprising crossing the soybean plant of any one of embodiments 1-5 with a second soybean plant to produce a progeny seed.

In accordance with embodiment 7 the method of any one of embodiments 1-6 is provided, wherein the progeny seed comprises the targeted genetic modification and a plant produced from the seed has increased resistance to at least one race of Phytophthora.

In accordance with embodiment 8 the method of any one of embodiments 1-7 is provided, wherein the second soybean plant is susceptible to the at least one race of Phytophthora.

In accordance with embodiment 9 the method of any one of embodiments 1-9 is provided, wherein the the plant, seed or plant produced from the seed has increased resistance to at least one race of Phytophthora selected from Race1, Race 2, Race3, Race4, Race5, Race6, Race7, Race8, Race9 Race13, Race17, Race25, or Race31.

In accordance with embodiment 10 a method for generating a Phytophthora resistant soybean plant is provided, the method comprising:
(a) introducing in a regenerable soybean plant cell a polynucleotide encoding a polypeptide comprising an amino acid sequence having at least 80% sequence identity to any one of SEQ ID NOs: 2 or 4, and
(b) generating the plant wherein the plant expresses the polynucleotide and has increased resistance to Phytophthora as compared to a control plant not expressing the polynucleotide.

In accordance with embodiment 11 the method of embodiment 10 is provided, wherein the regenerable plant cell is isolated from a soybean plant susceptible to at least one race of Phytophthora and the plant generated has increased resistance to the at least one race of Phytophthora.

In accordance with embodiment 12 the method of embodiment 10 or 11 is provided, wherein the at least one race of Phytophthora is at least one of Race1, Race 2, Race3, Race4, Race5, Race6, Race7, Race8, Race9 Race13, Race17, Race25, Race31.

In accordance with embodiment 13 the method of any one of embodiments 10-12 is provided, wherein the polynucleotide is introduced in the regenerable soybean plant cell using a targeted genetic modification.

In accordance with embodiment 14 the method of embodiment 13 is provided, wherein the targeted genetic modification is introduced using a genome modification technique selected from the group consisting of polynucleotide-guided endonuclease, CRISPR-Cas endonucleases, based editing deaminases, a zinc finger nuclease, a transcription activator-like effector nuclease (TALEN), engineered site-specific meganucleases, or Argonaute.

In accordance with embodiment 15 the method of any one of embodiments 10-14 is provided, wherein the polynucleotide is introduced in the regenerable soybean plant cell by a recombinant DNA construct comprising the polynucleotide operably linked to at least one regulatory element.

In accordance with embodiment 16 the method of any one of embodiments 10-15 is provided, wherein the at least one regulatory element is a promoter.

In accordance with embodiment 17 the method of embodiment 17 is provided, wherein the promoter is a heterologous promoter.

In accordance with embodiment 18 a method for increasing resistance to Phytophthora infection in a soybean plant, the method comprising:
(a) introducing in a regenerable soybean plant cell a targeted genetic modification increasing the expression of a polynucleotide encoding a polypeptide comprising an amino acid sequence having at least 80% sequence identity to any one of SEQ ID NOs: 2 or 4, and
(b) generating the plant wherein the plant has increased expression of the polynucleotide and increased resistance to Phytophthora infection as compared to a control plant not comprising the targeted genetic modification.

In accordance with embodiment 19 the method of embodiment 18 is provided, wherein the targeted genetic modification is introduced using a genome modification technique selected from the group consisting of polynucleotide-guided endonuclease, CRISPR-Cas endonucleases, based editing deaminases, a zinc finger nuclease, a transcription activator-like effector nuclease (TALEN), engineered site-specific meganucleases, or Argonaute.

In accordance with embodiment 20 the method of embodiment 18 or 19 is provided, wherein the method comprises:
(a) providing a guide RNA, at least one polynucleotide modification template, and at least one Cas endonuclease to a soybean plant cell, wherein the at least one Cas endonuclease introduces a double strand break at a locus in the soybean plant cell, and wherein the polynucleotide modification template inserts a polynucleotide encoding a polypeptide comprising an amino acid sequence that is at least 80% identical to any one of SEQ ID NOs: 2 or 4;
(b) obtaining a soybean plant from the soybean plant cell; and
(c) generating a progeny plant that comprises the polynucleotide and has increased resistance to Phytophthora as compared to a control pant not comprising the targeted genetic modification.

In accordance with embodiment 21 the method of any one of embodiments 18-20 is provided, wherein the locus is an endogenous Rps locus.

In accordance with embodiment 22 the method of any one of embodiments 18-21, wherein the Cas endonuclease is Cas9.

In accordance with embodiment 23 the method of any one of embodiments 18-22 is provided, wherein the plant cell is isolated from a soybean plant susceptible to at least one race of Phytophthora and the progeny plant has increased resistance to the at least one race of Phytophthora.

In accordance with embodiment 24 the method of embodiment 23 is provided, wherein the at least one race of Phytophthora is at least one of Race1, Race 2, Race3, Race4, Race5, Race6, Race7, Race8, Race9 Race13, Race17, Race25, Race31.

In accordance with embodiment 25 a method for producing a soybean plant with increased resistance to Phytophthora, the method comprising:
(a) expressing in a regenerable plant cell a polynucleotide encoding a polypeptide comprising an amino acid sequence having at least 80% sequence identity to any one of SEQ ID NOs: 2 or 4, and
(b) generating a plant wherein the plant expresses the polynucleotide and has increased resistance to Phytophthora infection as compared to a control plant.

In accordance with embodiment 26 the method of embodiment 25 is provided, wherein the method comprises:
(a) providing a guide RNA, at least one polynucleotide modification template, and at least one Cas endonuclease to a soybean plant cell, wherein the at least one Cas endonuclease introduces a double strand break in the genome of the soybean plant cell, and wherein the polynucleotide modification template inserts a polynucleotide encoding a polypeptide comprising an amino acid sequence that is at least 80% identical to any one of SEQ ID NOs: 2 or 4;
(b) obtaining a soybean plant from the soybean plant cell; and
(c) generating a progeny plant that comprises the polynucleotide and has increased resistance to Phytophthora as compared to a control pant not comprising the targeted genetic modification.

In accordance with embodiment 27 the method of embodiment 18-26 is provided, wherein the double strand break is introduced at an endogenous Rps gene in the soybean plant cell.

In accordance with embodiment 28 the method of any one of embodiments 18-27 is provided, wherein the Cas endonuclease is Cas9.

In accordance with embodiment 29 the method of any one of embodiments 18-28 is provided, wherein the plant cell is isolated from a soybean plant susceptible to at least one race of Phytophthora and the progeny plant has increased resistance to the at least one race of Phytophthora.

In accordance with embodiment 30 the method of embodiment 29 is provided, wherein the at least one race of Phytophthora is at least one of Race1, Race 2, Race3, Race4, Race5, Race6, Race7, Race8, Race9 Race13, Race17, Race25, Race31.

In accordance with embodiment 31 a soybean plant or soybean seed comprising a recombinant DNA construct comprising a polynucleotide encoding a polypeptide comprising an amino acid sequence that is at least 80% identical to any one of SEQ ID NOs: 2 or 4, wherein the soybean seed or soybean plant has increased expression of the polynucleotide as compared to a control plant not comprising the polynucleotide.

In accordance with embodiment 32 the soybean plant or soybean seed of embodiment 31 is provided, wherein the polynucleotide is operably linked to the endogenous promoter.

In accordance with embodiment 33 the soybean plant or soybean seed of embodiment 31 or 32 is provided, wherein the soybean plant or a plant grown from the soybean seed comprising the recombinant DNA construct has improved Phytophthora resistance as compared to the control plant.

In accordance with embodiment 34 a plant produced by the soybean seed of any one of embodiments 31-33 is provided.

In accordance with embodiment 35 a method of plant breeding is provided comprising crossing the soybean plant of any one of embodiments 31-34 with a second soybean plant to produce a progeny seed.

In accordance with embodiment 36 the method of embodiment 35 is provided, wherein the second soybean plant is susceptible to at least one race of Phytophthora and a plant produced from the progeny seed is resistant or has increased resistance to the at least one race of Phytophthora.

In accordance with embodiment 37 a method for increasing resistance to Phytophthora infection in a soybean plant, the method comprising:
(a) expressing in a regenerable soybean plant cell a recombinant DNA construct comprising a polynucleotide encoding a polypeptide comprising an amino acid sequence having at least 80% sequence identity to any one of SEQ ID NOs: 2 or 4, and
(b) generating the plant wherein the plant has increased expression of the polynucleotide and increased resistance to Phytophthora infection as compared to a control plant not comprising the recombinant DNA construct.

In accordance with embodiment 38 the method of embodiment 37 is provided, wherein the polynucleotide is operably linked to the endogenous promoter.

In accordance with embodiment 39 a method for identifying a soybean plant that displays increased resistance to Phytophthora is provided, the method comprising detecting in a soybean plant or seed thereof a polynucleotide sequence encoding a polypeptide comprising an amino acid sequence that is at least 80% identical to any one of SEQ ID NOs: 2 or 4.

In accordance with embodiment 40 the method of embodiment 39 is provided, wherein the polynucleotide sequence is detected using a primer comprising a nucleotide sequence of any one of SEQ ID NOs: 12-19.

In accordance with embodiment 41 The method of embodiment 39 or 40, further comprising:
a. obtaining a first soybean plant comprising the polynucleotide;
b. crossing the first soybean plant to a second soybean plant;
c. evaluating the progeny for the at least one allele; and
d. selecting progeny plants that possess the at least one allele thereby selecting plants with increased Phytophthora resistance.

In accordance with embodiment 42 a method for identifying a soybean plant that displays increased resistance to Phytophthora, the method comprising detecting in a soybean plant or seed thereof at least one allele of a marker locus associated with Rps11, Rps2b, Rps15, or Rps14.

In accordance with embodiment 43 the method of embodiment 42 is provided, wherein:
e. the marker locus is located within a chromosomal interval comprising and flanked by SSR-07-286 and SSR-07-320;
f. the marker locus is located within a chromosomal interval comprising and flanked by InDel3437 and InDel3701;
g. the marker locus is located within a chromosomal interval comprising and flanked by Satt431 and BARCSOY 16_1322; and
h. the marker locus is located within a chromosomal interval comprising and flanked by Satt631 and BARCSOY 03_0266.

In accordance with embodiment 44 the method of embodiment 42 or 43 is provided, further comprising:
i. obtaining a first soybean plant comprising the at least one allele of the marker locus;
j. crossing the first soybean plant to a second soybean plant;
k. evaluating the progeny for the at least one allele; and
l. selecting progeny plants that possess the at least one allele thereby selecting plants with increased Phytophthora resistance.

### Example 1

This example demonstrates the determination of the spectrum of resistance of Rps11 to Phytophthora sojae.

To explore the resistance spectrum of Rps11, a sub population derived from the Rps11 donor line (PI 594527) and Williams, including 14 lines with Rps11 and 14 lines without Rps11, were inoculated with 16 isolates of P. sojae.

The mapping populations were generated from an initial cross between PI 594527 and Williams. In 2015, 2640 F₃ plants derived from heterozygous F2 individuals were screened for the identification of recombinants. In 2016 and 2017, two additional larger populations of 7680 and 6730 F4 plants, respectively, derived from heterozygous F3 individuals, were screened for additional recombinants.

About 30 seedlings from each recombinant were inoculated with various isolates of Phytophthora sojae using a protocol previously described (Dorrance et al. 2008; Lin et al. 2013). Recombinants, in which less than 25% of the progenies survived after inoculation, were classified as susceptible; recombinants, with more than 25% progeny survival, were classified as segregation. The evaluation was repeated twice for each recombinant. For the resistance spectrum test, lines in which less than 25% of the progenies survived after inoculation were classified as susceptible, lines in which 25% to 75% of the progenies survived after inoculation were classified as partially resistant, and lines in which more than 75% of the progenies survived after inoculation were classified as completely resistant.

As shown in Table 2 all the lines with Rps11 were resistant to all 15 isolates, while all lines without Rps11 were susceptible to 14 isolates except Race 31, suggesting that Rps11 was resistant to all 15 isolates and another gene in the donor line was specifically resistant to Race 31 (Table 2).

TABLE 2
Resistance Spectrum of Rps11

| | Rps11^{a} | | | | | | |
|---|---|---|---|---|---|---|---|
| Sample | genotype | Race4^{b} | Race7 | Race25 | Race3 | Race31 | OH001 |
| 1 | rps11 | S | S | S | S | S | S |
| 2 | rps11 | S | S | S | S | S | S |
| 3 | rps11 | S | S | S | S | H | S |
| 4 | rps11 | S | S | S | S | R | S |
| 5 | rps11 | S | S | S | S | H | S |
| 6 | rps11 | S | S | S | S | H | S |
| 7 | rps11 | S | S | S | S | S | S |
| 8 | rps11 | S | S | S | S | R | S |
| 9 | rps11 | S | S | S | S | H | S |
| 10 | rps11 | S | S | S | S | H | S |
| 11 | rps11 | S | S | S | S | R | S |
| 12 | rps11 | S | S | S | S | H | S |
| 13 | rps11 | S | S | S | S | S | S |
| 14 | rps11 | S | S | S | S | H | S |
| 15 | RPS11 | R | R | R | R | R | R |
| 16 | RPS11 | R | R | R | R | R | R |
| 17 | RPS11 | R | R | R | R | R | R |
| 18 | RPS11 | R | R | R | R | R | R |
| 19 | RPS11 | R | R | R | R | R | R |
| 20 | RPS11 | R | R | R | R | R | R |
| 21 | RPS11 | R | R | R | R | R | R |
| 22 | RPS11 | R | R | R | R | R | R |
| 23 | RPS11 | R | R | R | R | R | R |
| 24 | RPS11 | R | R | R | R | R | R |
| 25 | RPS11 | R | R | R | R | R | R |
| 26 | RPS11 | R | R | R | R | R | R |
| 27 | RPS11 | R | R | R | R | R | R |
| 28 | RPS11 | R | R | R | R | R | R |

| | | | MIN1 | MIN1 | MIN1 | MIN1 | MIN1 | MIN1 |
|---|---|---|---|---|---|---|---|---|
| | | | 2004 | 2004 | 2002 | 2002 | 2005 | 2002 |
| Sample | OHC2S1 | OH003 | Mar. 1 | Jan. 1 | Jan. 5 | May 1 | Jul. 2 | May 5 |
| 1 | S | S | S | S | S | S | S | S |
| 2 | S | S | S | S | S | S | S | S |
| 3 | S | S | S | S | S | S | S | S |
| 4 | S | S | S | S | S | S | S | S |
| 5 | S | S | S | S | S | S | S | S |
| 6 | S | S | S | S | S | S | S | S |
| 7 | S | S | S | S | S | S | S | S |
| 8 | S | S | S | S | S | S | S | S |
| 9 | S | S | S | S | S | S | S | S |
| 10 | S | S | S | S | S | S | S | S |
| 11 | S | S | S | S | S | S | S | S |
| 12 | S | S | S | S | S | S | S | S |
| 13 | S | S | S | S | S | S | S | S |
| 14 | S | S | S | S | S | S | S | S |
| 15 | R | R | R | R | R | R | R | R |
| 16 | R | R | R | R | R | R | R | R |
| 17 | R | R | R | R | R | R | R | R |
| 18 | R | R | R | R | R | R | R | R |
| 19 | R | R | R | R | R | R | R | R |
| 20 | R | R | R | R | R | R | R | R |
| 21 | R | R | R | R | R | R | R | R |
| 22 | R | R | R | R | R | R | R | R |
| 23 | R | R | R | R | R | R | R | R |
| 24 | R | R | R | R | R | R | R | R |
| 25 | R | R | R | R | R | R | R | R |
| 26 | R | R | R | R | R | R | R | R |
| 27 | R | R | R | R | R | R | R | R |
| 28 | R | R | R | R | R | R | R | R |

^{a}RPS11 represents homozygous Rps11 genotype, rps11 means homozygous Williams genotype (No Rps11)
^{b}S represents susceptible, R represents resistant, H represents heterozygous (segregation)

These results demonstrated that Rps11 has a broad resistance spectrum to Phytophthora sojae.

### Example 2

This example demonstrates the identification of the Rps11 sequence.

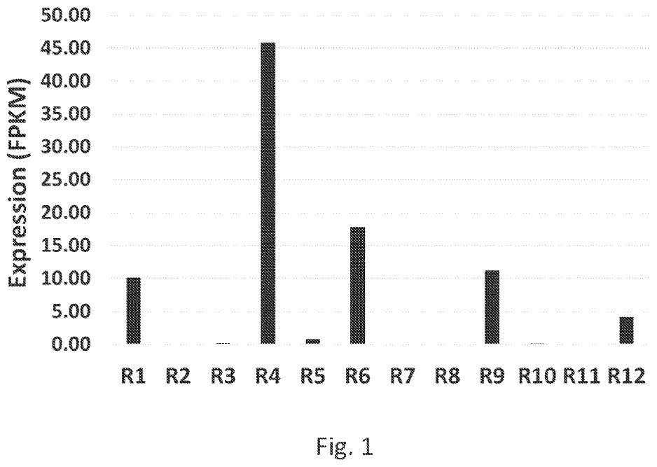

Whole genome sequencing and assembly was performed to obtain an assembly of the Rps11 region in the donor line. The genome was built with 34 kb PacBio reads which assembled into 424 contigs with a contig N50 of 13.8 Mb. These contigs were polished with PacBio and Chromium 10X data. The contigs were scaffolded with 45 BioNano maps with a map N50 of 26.7 Mb into 43 hybrid scaffolds with a scaffold N50 of 26.4 Mb, essentially 1-2 scaffolds per chromosome. Gene annotation identified 510 NBS-LRR genes across the entire genome, and twelve NBS-LRR genes were annotated at the Rps11 region, designated as R1 to R12. However, only five (R1, R4, R6, R9 and R12) out of the twelve NBS-LRR genes at Rps11 region were expressed based on RNA-seq analysis (FIG. 1).

In order to fine map the gene underlying Rps11 locus, 17,050 progenies derived from heterozygous F₂ or F₃ individuals were screened using flanking markers. In total, 43 recombinants were identified, and seedlings derived from these recombinants were inoculated with Race 1 isolate. The combination of additional markers and phenotypic data mapped Rps11 to a 151 kb genome interval, defined by maker_176 kb and InDel_327 kb, harboring 4 intact NBS-LRR genes (R5, R6, R7 and R8), but only R6 was expressed during inoculation, indicating R6 was the best candidate gene responsible for the resistance of Rps11.

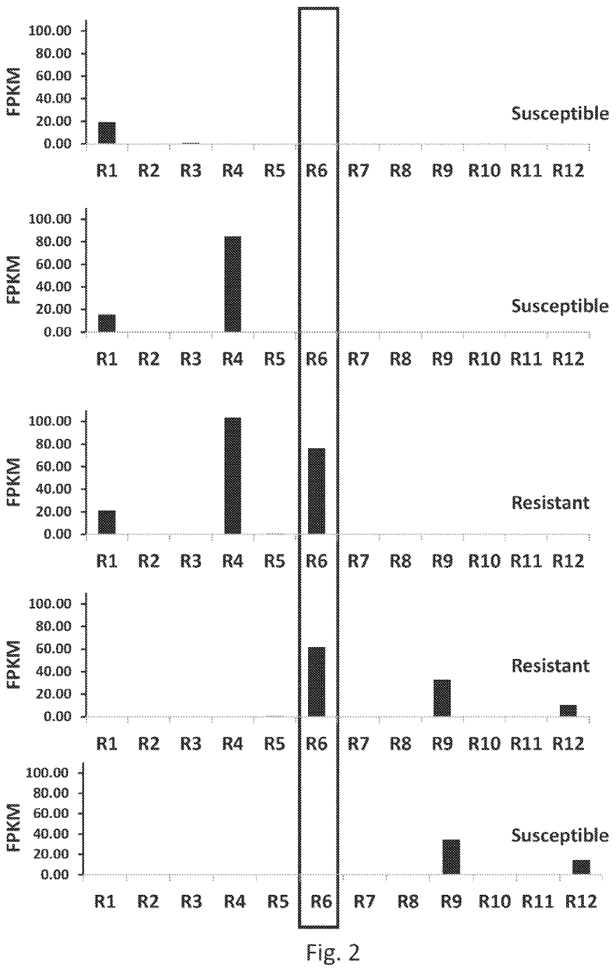

Expression of the NBS-LRR genes was further examined in 9 key recombinants that have different combinations of NBS-LRR genes. These studies determined that recombinants with R1 alone, a combination of R1 and R4, or a combination of R9 and R12 were susceptible, whereas the recombinants carrying R6 were resistant (FIG. 2).

These data indicate that R6, which encodes an NBS-LRR protein composed of 2,463 amino acids, is the gene underlying Rps11 locus.

### Example 3

This example demonstrates expression of the Rps11 in plants to increase resistance to Phytophthora sojae.

Gene constructs of the Rps11 candidate gene, R6 were made using the coding sequence of R6 (SEQ ID NO: 1) from the Rps11 donor line (PI 594527) for overexpression in soybean plants using the Ochrobactrum-mediated soybean embryonic axis transformation as previously described (US20180216123A1; WO2020/005933A1; WO2020/092494A1). The R6 gene construct was transformed into a proprietary soybean line that did not carry the Rps11 gene. Single copy TO transgenic events were selected based on the PCR analysis with primers specific to the cloning vector. T1 seed was harvested for phenotypic confirmation and expression of the Rps11 in plants. Since the T1 plants are expected to segregate for the transgene the screening for Phytophthora sojae races was used as an initial confirmation of the expression of the Rps11 candidate gene.

About 20 seedlings from three T1 families were inoculated with four isolates of Phytophthora sojae. Each of these isolates were previously tested on the soybean line used for the transformation to confirm that this line is not resistant to these isolates. For the resistance test, lines with less than 25% seedling survival after inoculation were classified as susceptible, lines with 25% to 75% seedling survival after inoculation were classified as heterozygous, and lines with more than 75% seedling survival after inoculation were classified as resistant.

As shown in Table 3 all the T1 families for the Rps11 candidate transgene construct were segregating or resistant to all the isolates, while the transformable line (TG) without Rps11 was susceptible and the Rps11 donor line, PI 594527 was resistant to all the isolates, suggesting that the candidate gene R6 confers increased resistance expression in the soybean plants that were otherwise susceptible to the Phytophthora sojae isolates and is therefore the Rps11 gene (Table 3).

For final confirmation of the Rps11 transgene expression in plants for resistance to Phytophthora sojae, T2 seed was generated by selfing the T1 plants. Homozygous, hemizygous and null T2 plants for the Rps11 candidate transgene were selected based on the PCR analysis. Phenotypic screening of the T2 plants was done using 3 different Phytophthora sojae isolates, 25, 31 and 06-03, using same method as was done for the T1 screening. As shown in Table 4, the phenotypic screening results showed significantly higher survival rate in homozygous plants as compared to the null plants with mean percent survival rate after inoculation with Phytophthora sojae isolate 31 ranging from 93.5% to 98.8% in homozygous plants and 0% to 11.33% in null plants. Similar level of Phytophthora disease resistance response was also observed when inoculated with isolates 31 and 06-03. Dosage effect of the R6 transgene was also observed with hemizygous plants showing intermediate level of resistance as compared to the homozygous and nulls (Table4). These results confirm that the expression of the Rps11 R6 gene provides increased resistance to Phytophthora sojae.

TABLE 3
Resistance response of T1 families to Phytophthora sojae isolates

| Genotype^{a} | R31 | R25 | 06-03 | OH002 |
|---|---|---|---|---|
| T1-Fam1 | NE | NE | H | H |
| T1-Fam2 | H | S^{b} | NE | NE |
| T1-Fam3 | R | R | R | H |
| TG | S | S | S | S |
| PI 594527 | R | R | R | R |

^{a}T1-Fam represents segregating genotype for Rps11 event; TG- Soybean line used for transformation
^{b}Single replication data for 6 plants only
^{c} S represents susceptible, R represents resistant, H represents heterozygous (segregation)

TABLE 4
Resistance response in T2 families to Phytophthora sojae isolates

| | | | Percent Survival Rate | | | |
|---|---|---|---|---|---|---|
| Event | N | Race | Homozygous | Hemizygous | Null | p-value |
| Event | 11 | 31 | 93.5^{A} (6) | 69.0^{A} (2) | 11.33^{B} (3) | 7.872e−06 |
| 1 | | 25 | 93.33^{A} | 82.0^{A} | 40.33^{B} | 5.02e−09 |
| | | 06-03 | 74.83^{A} | 61.50^{A} | 16.67^{B} | 2.29e−04 |
| Event | 13 | 31 | 93.71^{A} (7) | 56.67^{B} (3) | 0.00^{C} (3) | 1.395e−09 |
| 2 | | 25 | 97.43^{A} | 52.0^{A} | 26.67^{B} | 1.42e−07 |
| | | 06-03 | 85.0^{A} | 25.0^{B} | 2.33^{B} | 6.73e−09 |
| Event | 8 | 31 | 98.8^{A} (5) | NA | 8.33^{B} (3) | 7.306e−07 |
| 3 | | 25 | 92.2^{A} | NA | 15.0^{B} | 5.461e−05 |
| | | 06-03 | 84.6^{A} | NA | 0.00^{B} | 5.657e−06 |

N = total no. of T2 lines screened for each event. No. of lines in each genotypic class denoted in parenthesis for race 31. Same no. of lines in each genotypic class was screened for race 31 and 06-03.

### Example 4

This example demonstrates the identification of the Rps2b sequence.

PI 594549 C is a soybean landrace identified as a potential source of novel resistance to Phytophthora sojae in soybean according to USDA Germplasm Information Network (GRIN).

To determine the resistance of PI 594549 C to various races of Phytophthora sojae, disease evaluation was performed using the standard hypocotyl method for all the inoculation experiments. Briefly, 7-day-old seedlings growing in a greenhouse (˜25° C.) were tested by injecting 14-day-old Phytophthora sojae culture grown on ½ LBA into the hypocotyl of the seedling. In the first day of inoculation, trays holding injected soybean plants were covered by transparent plastic lids to maintain moisture for infection. Disease would further grow for another 5 to 7 days before scoring phenotypes.

For a single plant, if the plant continued to grow after inoculation, it was recorded as resistant, while susceptible would be counted for a dead brown hypocotyl seedling. For a single family, 12 to 36 seedlings were tested dependent on amounts of harvested seeds. If 75% or more seedlings in a family were resistant, then the family was classified as homozygous resistant (R), or homozygous susceptible(S) when less than 25% seedlings were resistant. The remaining families were counted as heterozygous resistant (Rs).

As shown in Table 5, the hypocotyl inoculation studies determined that PI 594594 C was resistant to all Phytophthora sojae isolates tested including race 1, race 3, race 4, race 7, race 13, race 17, race 25, four novel isolates from Minnesota (MIN12001.01.05, MIN12001.03.01, MIN12004.01.01 and MIN12005.07.02) and two novel isolates from Indiana (ISA19A-1, ISA71D-1).

TABLE 5
Responses of Soybean Landrace PI 5945493
to Different P. sojae Isolates

| | | No. of | No. of | |
|---|---|---|---|---|
| | Virulence | plants | plants | |
| P. sojae isolate | pathotype | examined | survived | Phenotype |
| Race 1 | 7 | 12 | 12 | Resistant |
| Race 3 | 1a, 7 | 9 | 10 | Resistant |
| Race 4 | 1a, 1c, 7 | 11 | 10 | Resistant |
| Race 7 | 1a, 2, 3a, 3c, 4, 5, | 10 | 10 | Resistant |
| | 6, 7 | | | |
| Race 13 | 4, 6, 7 | 11 | 9 | Resistant |
| Race 17 | 1b, 1d, 2, 3a, 3b, | 25 | 22 | Resistant |
| | 3c, 4, 5, 6, 7, 8 | | | |
| Race 25 | 1a, 1b, 1c, 1k, 7 | 10 | 9 | Resistant |
| ISA19A-1 | 1a, 1b, 1k, 4, 6, 7 | 10 | 10 | Resistant |
| ISA71D-1 | 1a, 1c, 1d, 7 | 11 | 10 | Resistant |
| MIN12001.01.05 | NA | 10 | 10 | Resistant |
| MIN12004.01.01 | NA | 10 | 10 | Resistant |
| MIN12004.03.01 | NA | 10 | 10 | Resistant |
| MIN12005.07.02 | NA | 11 | 11 | Resistant |

A single isolate ISA 124C-1 (race 1) was used to characterize the P. sojae resistance pattern found in PI 594549 C. By crossing PI 594549 C with the susceptible Williams cultivar, 173 F₂ individuals from self-pollination of F₁ were generated for a resistance test. For 59 F₂ individuals, 50 were resistant and 9 were susceptible, and the null hypothesis that resistance was carried by single dominant locus cannot be rejected (x²=2.89, p=0.09) (Table 6). Single locus inheritance pattern was further confirmed by evaluating the 104 F_{2:3} families against P. sojae race 1. The segregation of R (homozygous resistant): H (heterozygous resistant): S (homozygous susceptible) observed for resistance to race 1 was 26:48:30, which fits the expected 1:2:1 ratio (x²=0.92, p=0.63) (Table 6). In all, these results indicate that the resistance for P. sojae race 1 in PI 594549C is carried by a single Rps locus.

TABLE 6
Segregation Ratios of Phenotypes in the Mapping Population

| | | | | χ² goodness | | |
|---|---|---|---|---|---|---|
| | | | | of fit test | | |
| Parental lines and | Observed numbers | | | Expected | | |
| mapping population | R | Rs | S | ratio | χ² | p |
| PI 594549C | 12 | — | 0 | | | |
| Williams | 0 | — | 12 | | | |
| (PI 594549C × Williams) | 50 | — | 9 | 3:1 | 2.89 | 0.09 |
| F₂ plants | | | | | | |
| (PI 594549C × Williams) | 26 | 48 | 30 | 1:2:1 | 1.47 | 0.63 |
| F_{2:3} families | | | | | | |

R—homozygous resistant,
Rs—heterozygous resistant,
S—homozygous susceptible

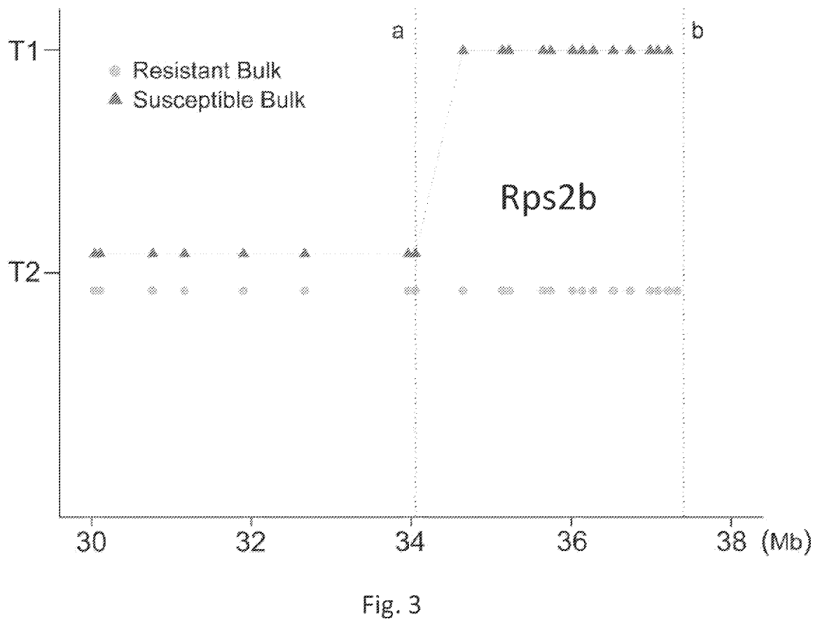

To detect chromosomal segments linked to the resistance gene for P. sojae race 1 in PI 594549C, ten resistant F_{2:3} families and ten susceptible F_{2:3} families were selected for bulked segregant analysis (BSA). Resistant and susceptible bulks were formed by pooling DNA samples of 10 resistant families and 10 susceptible families separately. The pooled samples were genotyped by the SoySNP6K BeadChip comprising 6210 effective SNP markers together with two parental lines. A total of 2,420 SNPs distributed along the 20 chromosomes were polymorphic between both parents. According to the principle of BSA, in the susceptible bulks, the SNP makers closely linked with the rps locus would be homozygous nucleotides that are the same as shown in the susceptible parental line Williams; whereas in the resistant bulk, such SNPs would be heterozygous nucleotides from both parental lines. Meanwhile, there will be no differences between resistant and susceptible bulks by the SNP markers unlinked with the Rps/rps locus and heterozygous nucleotides would be shown in both bulks. After comparison of the genotypes of the two bulks, a roughly 2-Mb region spanning from about 35 Mb to the end of chromosome 16 (FIG. 3) was found. This region also harbors a previously identified Rps2 and RpsUN2, thus the gene was designated as Rps2b (Rps2 Corteva Agriscience).

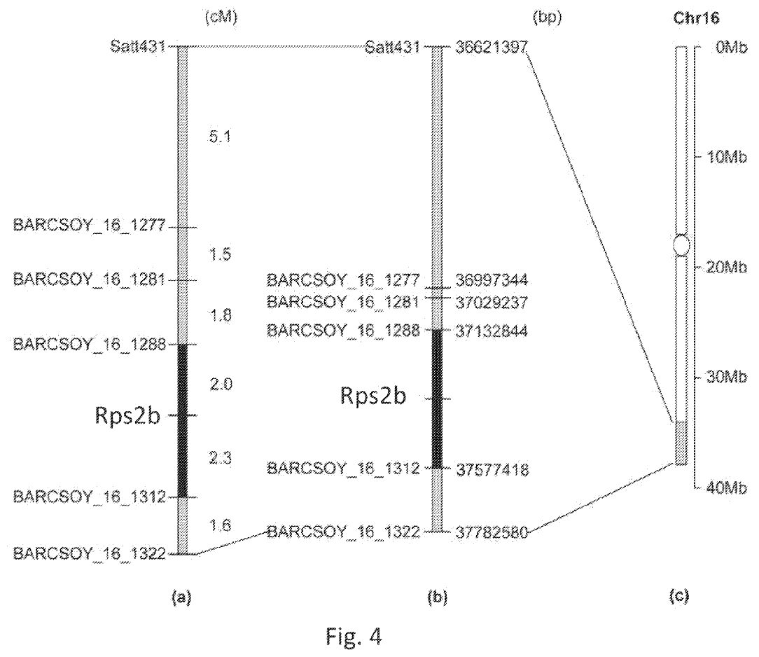

According to initial mapping using the BSA method, six polymorphic SSR markers Satt431 (SEQ ID NOs: 20 and 21), BARCSOYSSR_16_1277 (SEQ ID NOs: 22 and 23), BARCSOYSSR_12_1281 (SEQ ID NOs: 24 and 25), BARCSOYSSR_12_1288 (SEQ ID NOs: 26 and 27), BARCSOYSSR_16_1312 (SEQ ID NOs: 32 and 33), BARCSOYSSR_16_1322 (SEQ ID NOs: 34 and 35) from a total of 36 SSR markers were selected for the construction of a linkage map. All markers were observed at the expected 1:2:1 segregation ratio (Table 7), and 18, 11, 10, 9, 8 and 12 recombinants between each of these six markers and the Rps2b locus were defined, respectively. Among these 6 markers, Rps2b is more tightly linked with BARCSOYSSR_16_1288 and BARCSOYSSR_16_1312 compared with the rest of the four markers. A linkage map was constructed with these 6 makers and all markers were consistent with their order annotated on the Williams 82 reference genome (FIG. 4). On the map, Rps2b was defined to a 4.3-cM region flanked by BACSOYSSR_16_1288 and BARCSOYSSR_16_1312, and the physical distance between the two markers is about 444.6 kb.

TABLE 7
Chi-Square (χ²) Goodness of Fit Test for the
Markers in the F₂ Population Deduced Based on the
F_{2:3} Progenies Derived from PI 594549c × Williams

| | Observed number^{b} | | | χ² goodness of fit test | |
|---|---|---|---|---|---|
| Marker^{a} | a | h | b | χ²^{_{1:2:1}} | p |
| Satt431 | 30 | 46 | 28 | 1.50 | 0.48 |
| BARCSOYSSR_16_1288 | 26 | 47 | 31 | 1.44 | 0.49 |
| BARCSOYSSR_16_1294 | 26 | 47 | 31 | 1.44 | 0.49 |
| BARCSOYSSR_16_1297 | 23 | 51 | 30 | 4.39 | 0.11 |
| BARCSOYSSR_16_1312 | 26 | 49 | 28 | 0.98 | 0.61 |
| BARCSOYSSR_16_1322 | 26 | 50 | 28 | 0.23 | 0.89 |

^{a}SSR markers were obtained from Song et al. (2010)
^{b}a means homozygous for the marker allele from the resistant PI 594549C; b means homozygous for the marker allele from the susceptible Williams; h means heterozygous for the marker alleles from both parents

In order to narrow the mapping region of Rps2b, an F₃ population consisting of 1212 individuals and an F₄ population containing about 12,000 individuals were developed. Polymorphic KASP markers between PI 594549C and the Williams cultivar were developed from the soybean reference genome Wm82.al and were used for genotyping recombinants. The whole genome sequence of PI 594549C was sequenced by PacBio single molecule sequencing platform. No contig harbors both BARCSOYSSR_16_1288 and BARCSOYSSR_16_1312 in the sequencing library. A contig (tig14) harboring BARCSOYSSR_16_1288, BARCSOY, BARSCOYSSR_16_1294, BARCSOYSSR_16_1297 and BARCSOYSSR_16_1302 was used as a reference sequence for finer mapping of the Rps2b gene since it includes all R-genes in the mapping region. The KASP markers which can be uniquely mapped to tig14 were kept for accurate genotyping. There are 25 recombinants defined by BARCSOYSSR_16_1288 and BARCSOYSSR_16_1302, including 3 from the F₃ population and 22 from heterozygous-susceptible recombinants of the F₄ population. Phenotypes of these recombinants were identified from their derived families by hypocotyl inoculation methods. 2 recombinants (664, 7855) defined Rps2b downstream of KASP marker Gm16_36745870, while 7 recombinants (2123, 3600, 5303, 131-4, 10675, 5643, 31-1) defined the Rps2b upstream of KASP maker Gm16_36789567. For the remaining 16 recombinants, 15 of them were consistent with the 9 key recombinants, while the remaining recombinant defined Rps2b downstream of KASP marker Gm16_36789567. The physical distance between Gm16_36745870 and Gm 36789567 is 32.6 kb on tig14 and 7 genes are located is this region including 4 NBS type R-genes. Rps2 was mapped physically downstream of RpsUN2. Using RpsUN2 as a reference, Rps2b is also a different gene compared with Rps2, but likely to be a new locus of RpsUN2.

By genotyping with two flanking markers, BARCSOYSSR_16_1288 and BARCSOYSSR_16_1312, eight homozygous resistance families were tested with eight homozygous susceptible families as control (Table 8). Rps2b had resistance to 11 of 13 P. sojae isolates, and was susceptible to race 7 and race 17. By comparing Rps2 and RpsUN2, which is also mapped to the end of chromosome 16, Rps2b showed broader resistance as Rps2 showed complete resistance to only 7 of the isolates and RpsUN2 showed resistance to only 6 of the isolates. The results further indicate that Rps2b is a novel Rps gene.

TABLE 8
Marker Assisted Resistance Spectrum Analysis of Rps2b to Isolates of P. sojae

| P. sojae | | F_{2:3} families selected^{a} | | Parental lines | | Rps genes on Chr16 | |
|---|---|---|---|---|---|---|---|
| Isolate | Race | Rps2b | Rps2b | PI594549C | Williams | Rps2 | RpsUN2 |
| 124C-1 | 1 | R^{b} | S | R | S | R | R |
| 94-14-432(2) | 3 | R | S | R | S | R | R |
| 94-13p-197 | 4 | R | S | R | S | R | I |
| 95-11-117 (4) | 7 | S | S | R | S | S | S |
| pmg(13)-1 | 13 | R | S | R | S | S | I |
| pmg(17)-1 | 17 | S | S | R | S | S | S |
| pmg(25)-1 | 25 | R | S | R | S | R | R |
| ISA 19A-1 | N/A | R | S | R | S | I | I |
| ISA 71D-1 | N/A | R | S | R | S | I | I |
| MIN1 | N/A | R | S | R | S | R | R |
| 2001 Jan. 5 | | | | | | | |
| MIN1 | N/A | R | S | R | S | I | R |
| 2004 Jan. 1 | | | | | | | |
| MIN1 | N/A | R | S | R | S | R | R |
| 2004 Mar. 1 | | | | | | | |
| MIN1 | N/A | R | S | R | S | R | S |
| 2005 Jul. 2 | | | | | | | |

^{a}BARCSOYSSR_16_1288 and BARCSOYSSR_16_1312 are two molecular markers used for selections. The resistance pattern of Rps2b against each P. sojae isolate was scored by the proportion of resistance progenies in 8 selected homozygous resistant F_{2:3} families. The reaction of Rpscas was evaluated from 8 homozygous susceptible F_{2:3} families.
^{b}A family was recorded as resistant if >75% of seedlings survived after inoculation, susceptible if <25% of seedlings survived, and intermediate resistant if the proportion of resistant seedlings was between 25% and 75%.

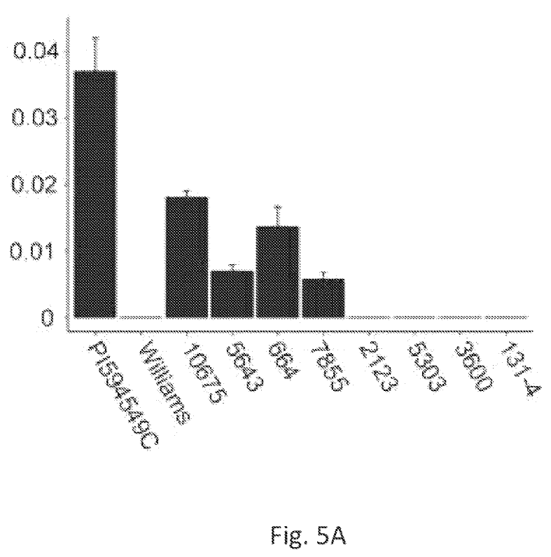

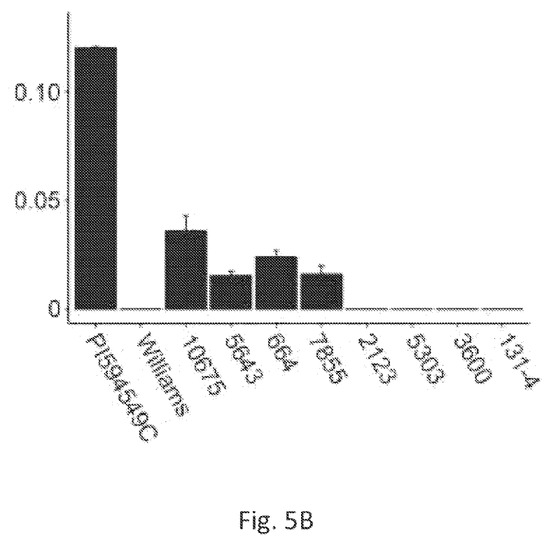

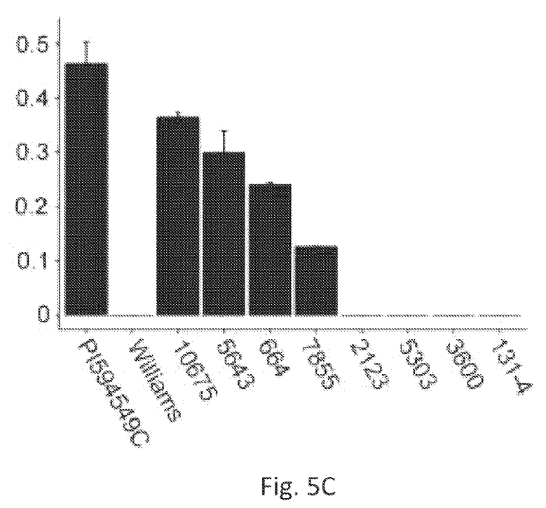

The first of the 4 NBS type R-gene in the fine-mapping region on tig14 was completely shared by PI 594549C and Williams with 100% sequence identity, while the remaining 3 R-genes were not found in the mapping region of Williams 82 reference genome. In recombinants whose phenotypes are susceptible, expression of these 3 genes is not detected, while for recombinants whose genotypes are heterozygous resistant, all these three genes are expressed (FIGS. 5A-5C).

The gene-structure of these three NBS type R-genes were further analyzed and of the three genes only R-gene3 is a complete TIR-NBS-LRR type R-gene. For Rgene2, though the length of its transcript is 4410 bp and shares 99% sequence identity with Glycine soja TMV resistance gene in newly sequenced wild soybean W05 genome, it has a premature stop codon. As a result, the length of its potential longest open reading frame is only 1926 bp which encodes incomplete 641-amino acid R gene carrying only a TIR-NBS domain. For Rgene4, the length of its longest ORF is 1152 bp and also encodes 383-amino acid R gene with only a TIR-NBS domain. Taken together these results indicate that Rgene3 is likely to be the candidate gene for Rps2b.

### Example 5

This example demonstrates expression of Rps2b in plants to increase resistance to Phytophthora sojae.

A gene construct comprising the Rps2b candidate gene (Rgene3) was made using the CDS of Rgene3 from the Rps2b donor line (PI 594549 C). The Rps2b gene construct was transformed into the susceptible Williams soybean line and single copy TO transgenic events were selected based on the PCR analysis with primers specific to the cloning vector. Two independent transgenic lines were obtained for further testing.

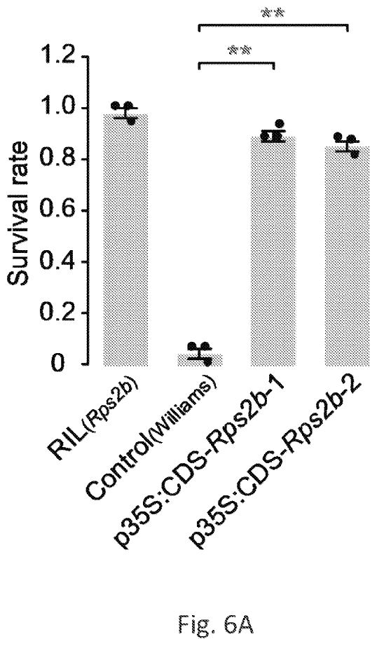

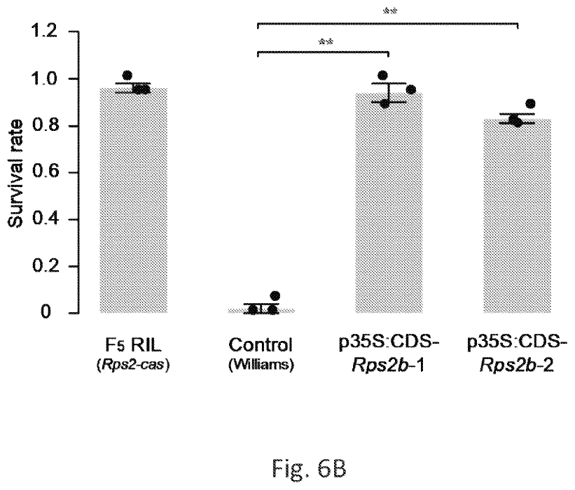

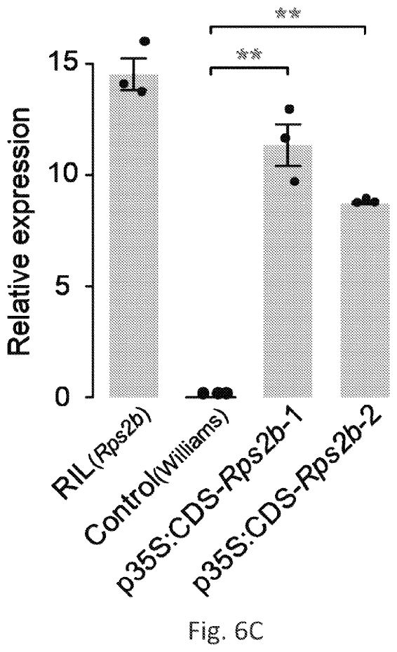

T2 plants expressing the Rps2b gene, the soybean line F₅ RIL (positive control line), and the native Williams line (negative control) were inoculated with P. sojae Race 1 and Race 25. As shown in FIGS. 6A and 6B, transgenic lines expressing the Rps2b gene had similar levels of resistance to Race 1 (FIG. 6A) and Race 25 (FIG. 6B) as compared with the F₅ RIL line. The expression level of Rps2b in the T2 transgenic lines were similar to the expression level of Rps2b in the F₅ RIL line (FIG. 6C), suggesting that the resistance does not result from autoimmunity triggered by the over-expression of an NBS-LRR gene.

Taken together, these results indicate that Rgene3 is the Rps2b gene. \

### Example 6

This example demonstrates the identification of the Rps15 sequence.

PI 594592 carries resistance to P. sojae races 1, 3, 7 and 25. Evaluation of P. sojae resistance/susceptibility to other Phytophthora sojae races was examined using a modified hypocotyl inoculation. 7-day-old soybean seedlings growing in a greenhouse (about 25° C.) were inoculated with mycelial slurry from 14-day-old isolates maintained on ½ LBA media. A small incision was made approximately 1 cm below the cotyledon for insertion of mycelium into the wound. After inoculation, each tray holding seedlings was covered with a transparent plastic lid for one day to create an environment with a high humidity level to promote infection. Before scoring phenotypes, disease growth was maintained for another 5-7 days after removal of the lid.

For a single plant, if the seedling kept growing after inoculation, it was recorded as resistant, and if the seedlings died with necrosis in the hypocotyl it was marked as susceptible. 20 to 36 progeny was analyzed for phenotype scoring for each family. A family was classified as homozygous resistant (R) if no less than 75% progenies survived, segregating (Rs) if 25-75% of progenies survived, or heterozygous susceptible(S) if less than 25% of the seedlings were alive.

As shown in Table 9, resistance to P. sojae races 1, 3, 7 and 25 was confirmed and it was further determined that PI 594592 carries resistance to P. sojae races 4, 13, 17, and two newly found predominant isolates collected from soybean fields in Indiana (ISA19A-1, ISA17D-1) and four isolates collected from soybean fields in Minnesota (MIN12001.01.05, MIN12004.01.01, MIN12004.03.01 and MIN12005.07.02).

TABLE 9
Responses of Soybean Landrace PI 594592
to Different P. sojae Isolates

| | | No. of | No. of | |
|---|---|---|---|---|
| | Virulence | plants | plants | |
| P. sojae isolate | pathotype | examined | survived | Phenotype |
| Race 1 | 7 | 10 | 10 | Resistant |
| Race 3 | 1a, 7 | 11 | 11 | Resistant |
| Race 4 | 1a, 1c, 7 | 10 | 10 | Resistant |
| Race 7 | 1a, 2, 3a, 3c, 4, 5, | 12 | 11 | Resistant |
| | 6, 7 | | | |
| Race 13 | 4, 6, 7 | 11 | 11 | Resistant |
| Race 17 | 1b, 1d, 2, 3a, 3b, | 12 | 11 | Resistant |
| | 3c, 4, 5, 6, 7, 8 | | | |
| Race 25 | 1a, 1b, 1c, 1k, 7 | 10 | 10 | Resistant |
| ISA19A-1 | 1a, 1b, 1k, 4, 6, 7 | 11 | 11 | Resistant |
| ISA71D-1 | 1a, 1c, 1d, 7 | 11 | 11 | Resistant |
| MIN12001.01.05 | NA | 10 | 10 | Resistant |
| MIN12004.01.01 | NA | 10 | 10 | Resistant |
| MIN12004.03.01 | NA | 11 | 11 | Resistant |
| MIN12005.07.02 | NA | 10 | 10 | Resistant |

To determine the inheritance pattern of P. sojae resistance in PI 594592, 60 F₂ plants derived from a cross between PI 594592 and the Williams cultivar were tested using race 1. Of the 60 plants, 46 were resistant to race 1, while 14 were susceptible. This observation suggests that resistance to P. sojae race 1 is carried by a single locus in PI 594592 since the segregation fits a phenotypic ratio of 3: 1 (x²=0.09, p=0.77) (Table 10). 200 F_{2:3} families from F₂ plants were subsequently developed by self-pollination, and tested for resistance to race 1. Among these 200 families, 47 were homozygous resistant (R), 107 showed heterozygous resistance, and 50 were homozygous susceptible, which fit a phenotypic ratio of 1:2:1 ((x²=0.09, p=0.77). This observation further indicates the resistance to P. sojae race1 is conferred by a single locus.

TABLE 10
Segregation Ratios of Phenotypes in the Mapping Population

| | | | | χ² goodness | | |
|---|---|---|---|---|---|---|
| | | | | of fit test | | |
| Parental lines and | Observed numbers | | | Expected | | |
| mapping population | R | Rs | S | ratio | χ² | p |
| PI 594592 | 12 | — | 0 | | | |
| Williams | 0 | — | 12 | | | |
| (PI 594592 × Williams) | 46 | — | 14 | 3:1 | 0.09 | 0.77 |
| F₂ plants | | | | | | |
| (PI 594592 × Williams) | 43 | 107 | 50 | 1:2:1 | 1.47 | 0.48 |
| F_{2:3} families | | | | | | |

R—homozygous resistant,
Rs—heterozygous resistant,
S—homozygous susceptible

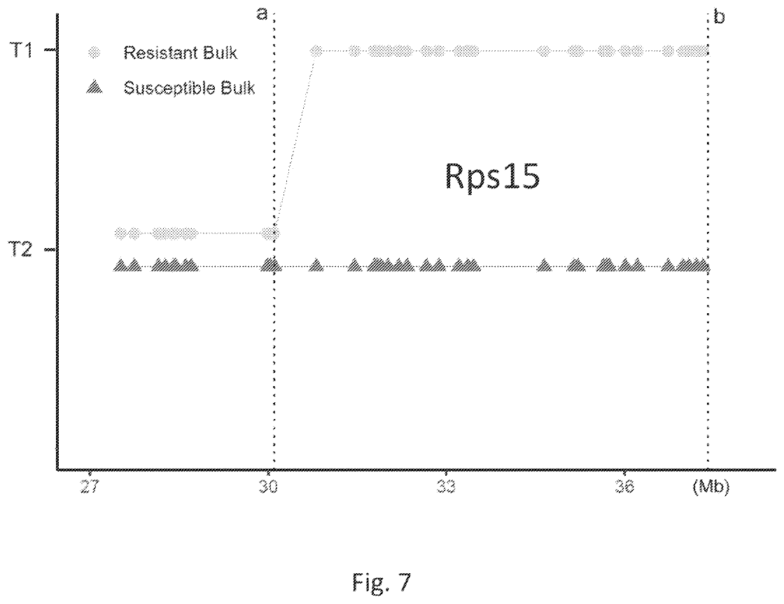

10 completely susceptible F_{2:3} families and 10 completely resistant F_{2:3} families were selected to make up susceptible and resistant bulks. The resistant and susceptible bulks, along with the two parental lines Williams and PI 594592, were genotyped with the SoySNP6K Infinium BeadChips consisting of 6000 SNP markers. In total, there are 2509 polymorphic SNPs distributed along all 20 chromosomes between the two parental lines. If there were differences detected between resistant and susceptible bulks, and the SNP markers detected homozygous nucleotides that are same as shown in the resistant parental line PI 594592, then the SNP markers are believed to closely linked to Rps locus. Based on this principle, a ˜6.5 Mb genomic region on chromosome 16 showing homozygous SNP sites in resistant bulk and heterozygous SNP sites in susceptible bulk was identified, starting from ˜30.8 Mb to the end of chromosome16 according to soybean reference genome Wm82.al (Table 11 and FIG. 7).

TABLE 11
Genotypic Comparison Between the Resistant and Susceptible
Bulks at the SNP Sites Detected Between the Two
Parental Lines at the End of Chromosome 16

| | | | | Suscep- | Resis- | |
|---|---|---|---|---|---|---|
| | Chromo- | Posi- | Wil- | tible | tant | |
| SNP ID | some | tion(bp) | liams | Bulk | Bulk | PI 594592 |
| SNP1912 | Gm16 | 28266706 | TT | TC | TC | CC |
| SNP1913 | Gm16 | 28407237 | AA | AC | AC | CC |
| SNP1914 | Gm16 | 28443553 | TT | TC | TC | CC |
| SNP1915 | Gm16 | 28613278 | CC | TC | TC | TT |
| SNP1916 | Gm16 | 28706800 | AA | AG | AG | GG |
| SNP1917 | Gm16 | 29985920 | AA | AG | AG | GG |
| SNP1918 | Gm16 | 30038668 | GC | TG | TG | TT |
| SNP1919 | Gm16 | 30108889 | CC | AC | AC | AA |
| SNP1920 | Gm16 | 30813568 | GG | TG | TT | TT |
| SNP1921 | Gm16 | 31454423 | GG | AG | AA | AA |
| SNP1922 | Gm16 | 31787658 | TT | TC | CC | CC |
| SNP1923 | Gm16 | 31837545 | CC | TC | TT | TT |
| SNP1924 | Gm16 | 31899513 | GG | AG | AA | AA |
| SNP1925 | Gm16 | 32017661 | AA | AC | CC | CC |
| SNP1926 | Gm16 | 32200441 | AA | AC | CC | CC |
| SNP1927 | Gm16 | 32340079 | GG | AG | AA | AA |
| SNP1928 | Gm16 | 32665742 | TT | TC | CC | CC |
| SNP1929 | Gm16 | 32876100 | AA | AG | GG | GG |
| SNP1930 | Gm16 | 33210540 | TT | TC | CC | CC |
| SNP1931 | Gm16 | 33360539 | TT | TC | CC | CC |
| SNP1932 | Gm16 | 33457667 | TT | TT | GG | GG |
| SNP1933 | Gm16 | 34645180 | AA | AG | GG | GG |
| SNP1934 | Gm16 | 35148803 | TT | TG | GG | GG |
| SNP1935 | Gm16 | 35218386 | GG | TG | TT | TT |
| SNP1936 | Gm16 | 35643452 | CC | TC | TT | TT |
| SNP1937 | Gm16 | 35700223 | GG | TG | TT | TT |
| SNP1938 | Gm16 | 35738081 | AA | AG | GG | GG |
| SNP1939 | Gm16 | 36013043 | AA | AC | AA | CC |
| SNP1940 | Gm16 | 36217195 | TT | TC | CC | CC |
| SNP1941 | Gm16 | 36732450 | TT | TT | CC | CC |
| SNP1942 | Gm16 | 36983033 | CC | CC | AA | AA |
| SNP1943 | Gm16 | 37078478 | AA | AA | GG | GG |
| SNP1944 | Gm16 | 37209075 | TT | TT | GG | GG |

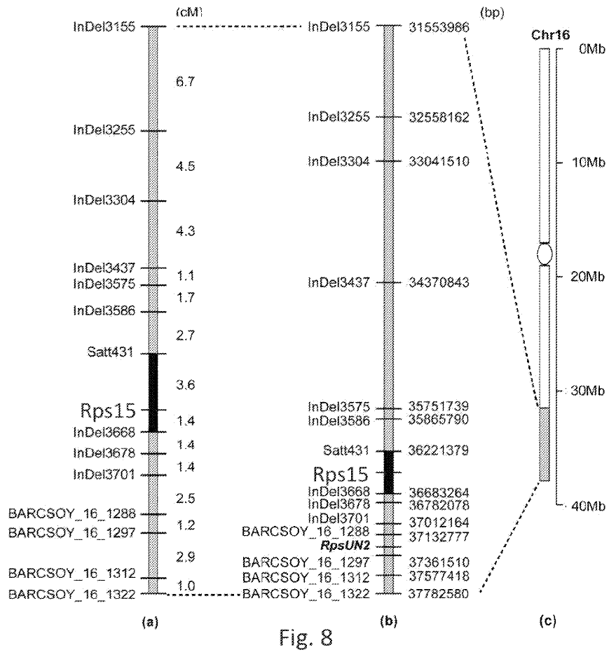

In order to fine map the Rps gene, the re-sequencing data of the two parental lines combined with the reference genome was used in marker design. 10 InDel markers in the defined region were designed and confirmed for polymorphism between the two parental lines. 6 potential SSR markers that are closely linked with two previously identified Rps genes, Rps2 and RpsUN2 were also tested. These markers also showed polymorphism between the two parental lines (Table 12). These 16 markers are evenly distributed within the defined ˜6.5 Mb Rps15 region and were used for genotyping 200 F_{2:3} families. Each of these markers revealed a 1:2:1 ratio for three possible genotypes (R, Rs, S) in the mapping population (Table 13). Combining all the genotypic and phenotypic data from the F_{2:3} families, a linkage map comprising all the sixteen markers in the Rps15 locus (FIG. 8) was constructed. In the map, Rps15 is flanked by SSR marker Satt43land InDel marker InDel3618, and the genetic distance to Rps15 were 3.6 and 1.4 cM, respectively. According to the soybean Williams82 reference genome, the 5 cM region corresponds to a roughly 462 kb region harboring 58 different genes (Table 14).

TABLE 12
Polymorphic Insertion/Deletion (InDel) and SSR Markers
Identified Between PI594592 and Williams

| SSR Marker | Chr | Forward primer | Reverse primer |
|---|---|---|---|
| InDel3155 | 16 | SEQ ID NO: 86 | SEQ ID NO: 87 |
| InDel3255 | 16 | SEQ ID NO: 88 | SEQ ID NO: 89 |
| InDel3304 | 16 | SEQ ID NO: 90 | SEQ ID NO: 91 |
| InDel3437 | 16 | SEQ ID NO: 92 | SEQ ID NO: 93 |
| InDel3575 | 16 | SEQ ID NO: 94 | SEQ ID NO: 95 |
| InDel3586 | 16 | SEQ ID NO: 96 | SEQ ID NO: 97 |
| InDel3668 | 16 | SEQ ID NO: 98 | SEQ ID NO: 99 |
| InDel3678 | 16 | SEQ ID NO: 100 | SEQ ID NO: 101 |
| InDel3701 | 16 | SEQ ID NO: 102 | SEQ ID NO: 103 |
| Satt431 | 16 | SEQ ID NO: 20 | SEQ ID NO: 21 |
| BARCSOYSSR_16_1288 | 16 | SEQ ID NO: 26 | SEQ ID NO: 27 |
| BARCSOYSSR_16_1294 | 16 | SEQ ID NO: 28 | SEQ ID NO: 29 |
| BARCSOYSSR_16_1297 | 16 | SEQ ID NO: 30 | SEQ ID NO: 31 |
| BARCSOYSSR_16_1312 | 16 | SEQ ID NO: 32 | SEQ ID NO: 33 |
| BARCSOYSSR_16_1322 | 16 | SEQ ID NO: 34 | SEQ ID NO: 35 |

TABLE 13
Chi-Square (χ²) Goodness of Fit Test for the
Markers in the F₂ Population Deduced Based on the
F_{2:3} Progenies Derived From Pi 594592 × Williams

| | Observed number^{a} | | | χ² goodness of fit test | |
|---|---|---|---|---|---|
| Marker | a | h | b | χ²^{_{1:2:1}} | p |
| | 43 | 107 | 50 | 1.47 | 0.48 |
| InDel3155 | 48 | 103 | 49 | 0.19 | 0.91 |
| InDel3255 | 50 | 98 | 52 | 0.12 | 0.94 |
| InDel3304 | 47 | 106 | 47 | 0.72 | 0.70 |
| InDel3437 | 48 | 105 | 47 | 0.51 | 0.77 |
| InDel3575 | 44 | 109 | 47 | 1.71 | 0.43 |
| InDel3586 | 44 | 109 | 47 | 1.71 | 0.43 |
| Satt431 | 47 | 104 | 49 | 0.36 | 0.84 |
| InDel3668 | 45 | 106 | 49 | 0.88 | 0.64 |
| InDel3678 | 45 | 109 | 46 | 1.63 | 0.44 |
| InDel3701 | 43 | 110 | 47 | 2.16 | 0.34 |
| BARCSOYSSR_16_1288 | 40 | 113 | 47 | 3.87 | 0.14 |
| BARCSOYSSR_16_1294 | 41 | 113 | 46 | 3.63 | 0.16 |
| BARCSOYSSR_16_1297 | 41 | 112 | 47 | 3.24 | 0.20 |
| BARCSOYSSR_16_1312 | 42 | 112 | 46 | 3.04 | 0.22 |
| BARCSOYSSR_16_1322 | 44 | 110 | 46 | 2.04 | 0.36 |

^{a}a means homozygous for the marker allele from the resistant PI 594592; b means homozygous for the marker allele from the susceptible Williams; h means heterozygous for the marker alleles from both parents

TABLE 14
Gene Annotation in the Mapped Region According
to the Soybean Reference Genome (Wm82.a2.v1)

| Genes | Annotation |
|---|---|
| Glyma.16g200900 | MEMBER OF ‘GDXG’ FAMILY OF LIPOLYTIC ENZYMES |
| Glyma.16g201000 | MEMBER OF ‘GDXG’ FAMILY OF LIPOLYTIC ENZYMES |
| Glyma.16g201100 | Helix-loop-helix DNA-binding domain |
| Glyma.16g201200 | LEUCINE-RICH REPEAT RECEPTOR-LIKE PROTEIN KINASE |
| Glyma.16g201300 | Helix-loop-helix DNA-binding domain |
| Glyma.16g201400 | Helix-loop-helix DNA-binding domain |
| Glyma.16g201500 | LEUCINE-RICH REPEAT RECEPTOR-LIKE PROTEIN KINASE |
| Glyma.16g201600 | EXOSTOSIN (HEPARAN SULFATE GLYCOSYLTRANSFERASE)-RELATED |
| Glyma.16g201700 | PROPROTEIN CONVERTASE SUBTILISIN/KEXIN |
| Glyma.16g201800 | Transferase family |
| Glyma.16g201900 | cysteine-rich RLK (RECEPTOR-like protein kinase) 25 |
| Glyma.16g202000 | Embryo-specific protein 3 |
| Glyma.16g202100 | cysteine-rich RLK (RECEPTOR-like protein kinase) 25 |
| Glyma.16g202200 | LEUCINE-RICH REPEAT RECEPTOR-LIKE PROTEIN KINASE |
| Glyma.16g202300 | HXXXD-type acyl-transferase family protein |
| Glyma.16g202400 | LEUCINE-RICH REPEAT RECEPTOR-LIKE PROTEIN KINASE |
| Glyma.16g202500 | Embryo-specific protein 3 |
| Glyma.16g202600 | F-box family protein |
| Glyma.16g202700 | 40S RIBOSOMAL PROTEIN S14/30S RIBOSOMAL PROTEIN S11 |
| Glyma.16g202800 | F-box family protein |
| Glyma.16g202900 | F-box family protein |
| Glyma.16g203000 | F-box family protein |
| Glyma.16g203100 | Iron-binding zinc finger CDGSH type |
| Glyma.16g203200 | POTASSIUM/PROTON ANTIPORTER-RELATED |
| Glyma.16g203300 | LEUCINE-RICH REPEAT RECEPTOR-LIKE PROTEIN KINASE |
| Glyma.16g203400 | BOX C/D SNORNA PROTEIN 1 |
| Glyma.16g203500 | RING FINGER DOMAIN-CONTAINING |
| Glyma.16g203600 | 1-AMINOCYCLOPROPANE-1-CARBOXYLATE SYNTHASE |
| Glyma.16g203700 | GLUTAMATE DEHYDROGENASE/NADP-SPECIFIC GLUTAMATE |
| | DEHYDROGENASE 1-RELATED |
| Glyma.16g203800 | No functional annotations |
| Glyma.16g203900 | DNA REPAIR/RNA PROCESSING CPSF FAMILY/CLEAVAGE AND |
| | POLYADENYLATION SPECIFICITY FACTOR SUBUNIT 1 |
| Glyma.16g204000 | ATP-DEPENDENT CLP PROTEASE |
| Glyma.16g204100 | ATP-DEPENDENT CLP PROTEASE |
| Glyma.16g204200 | BED zinc finger/hAT family C-terminal dimerisation region |
| Glyma.16g204300 | PROTEIN REGULATOR OF CYTOKINESIS 1 PRC1-RELATED |
| Glyma.16g204400 | No functional annotations |
| Glyma.16g204500 | integral component of membrane |
| Glyma.16g204600 | Enolase, C-terminal TIM barrel domain/Enolase, N-terminal domain |
| Glyma.16g204700 | nucleic acid binding |
| Glyma.16g204800 | Tetratricopeptide repeat (TPR)-like superfamily protein |
| Glyma.16g204900 | SPFH domain/Band 7 family |
| Glyma.16g205000 | No functional annotation |
| Glyma.16g205100 | LEUCINE-RICH REPEAT RECEPTOR-LIKE PROTEIN KINASE |
| Glyma.16g205200 | CHLOROPHYLL A/B BINDING PROTEIN |
| Glyma.16g205300 | integral component of membrane |
| Glyma.16g205400 | No functional annotation |
| Glyma.16g205500 | APO PROTEIN 1, CHLOROPLASTIC |
| Glyma.16g205600 | No functional annotation |
| Glyma.16g205700 | AAA domain (Cdc48 subfamily)/C-terminal, D2-small domain, of ClpB protein |
| Glyma.16g205800 | No functional annotation |
| Glyma.16g205900 | No functional annotation |
| Glyma.16g206000 | Pentatricopeptide repeat (PPR) superfamily protein |
| Glyma.16g206100 | Trypsin and protease inhibitor |
| Glyma.16g206200 | Hsp20/alpha crystallin family |
| Glyma.16g206300 | Trypsin and protease inhibitor |
| Glyma.16g206400 | chloroplast relocation |
| Glyma.16g206500 | Ribosomal Proteins L2, C-terminal domain |
| Glyma.16g206600 | ATP binding; nucleic acid binding; helicases |

Previously, RpsUN2 was fine mapped downstream of BARCSOY_SSR_1288 and Rps15 is 7.7 cM upstream of the same marker. Meanwhile, Rps2 was mapped 12.2 cM downstream to Satt431, which means the genetic distance between Rps15 and Rps2 is about 8.6 cM. This result suggests that Rps15 is more likely to be a novel Rps gene rather than a new allele for Rps2 or RpsUN2.

Of the 13 isolates of P. sojae used, Rps15 conferred resistance to 12 isolates and only showed susceptibility to Race 7 (Table 15). Rps2 was resistant to 7 isolates, partially resistant to 4, and susceptible to 3 (Race7, Race 13 and Race 17). RpsUN2 was resistant to 6 isolates, with intermediate resistance to 4 and susceptibility to 4. (Race 7, Race 17 and MIN12005.07.02). These results indicated that Rps15 had a much broader resistance spectrum compared with Rps2 and RpsUN2.

TABLE 15
Marker Assisted Resistance Spectrum Analysis of Rps15 to Isolates of P. sojae

| P. sojae | | F_{2:3} families selected^{a} | | Parental lines | | Rps genes on Chr16 | |
|---|---|---|---|---|---|---|---|
| Isolate | Race | Rps15 | Rps15 | PI 594592 | Williams | Rps2 | RpsUN2 |
| 124C-1 | 1 | R^{b} | S | R | S | R | R |
| 94-14-432(2) | 3 | R | Segregation | R | S | R | R |
| 94-13p-197 | 4 | R | S | R | S | R | I |
| 95-11-117 (4) | 7 | S | S | R | S | S | S |
| pmg(13)-1 | 13 | R | Segregation | R | S | S | I |
| pmg(17)-1 | 17 | R | Segregation | R | S | S | S |
| pmg(25)-1 | 25 | R | S | R | S | R | R |
| ISA 19A-1 | N/A | R | S | R | S | I | I |
| ISA 71D-1 | N/A | R | S | R | S | I | I |
| MIN1 | N/A | R | S | R | S | R | R |
| 2001 Jan. 5 | | | | | | | |
| MIN1 | N/A | R | S | R | S | I | R |
| 2004 Jan. 1 | | | | | | | |
| MIN1 | N/A | R | S | R | S | R | R |
| 2004 Mar. 1 | | | | | | | |
| MIN1 | N/A | R | S | R | S | R | S |
| 2005 Jul. 2 | | | | | | | |

^{a}Satt431 and InDel3668 are two molecular markers used for selections. The resistance pattern of Rps15 against each P. sojae isolate was scored by the proportion of resistance progenies in 8 selected homozygous resistant F_{2:3} families. The reaction of Rps15 was evaluated from 8 homozygous susceptible F_{2:3} families.
^{b}A family was recorded as resistant if >75% of seedlings survived after inoculation, susceptible if <25% of seedlings survived, and intermediate resistant if the proportion of resistant seedlings was between 25% and 75%.

To further determine the candidate gene for Rps15, NBS type R-genes, such as those provided in Table 14, will be identified in the Rps15 locus and analyzed. The Rps15 genomic interval will be further fine mapped using a large F₃ mapping population derived from a cross between PI 594592 and the susceptible parental Williams cultivar. The mapping population will be genotyped with molecular markers to identify recombinant lines. The recombinants will be phenotyped with Phytophthora sojae isolate Race 1. Based on the fine mapping, the Rps15 QTL region will be narrowed down to a smaller genomic interval comprising a few candidate genes for further confirmation. To identify candidate genes for Rps15, a whole genome sequence of PI 594592 will be generated using the PacBio sequencing platform. A contig harboring the flanking markers for the Rps15 genomic interval will be used as a reference sequence for comparing to the sequence data from the susceptible lines, characterizing the genes and identifying candidate genes based on the NBS type R-genes as candidates for Rps15. The NBS type R-genes will be further analyzed for their gene structure to determine if it is a complete NBS type gene or truncated. Gene expression analysis will also be done to determine the expression profile of the NBS type R-genes in the recombinants. Together, with these analyses candidate genes will be expected to be identified for Rps15. Constructs comprising the candidate genes will be generated and transformed into soybean lines that do not carry the Rps15 candidate gene to confirm the Rps15 candidate gene and determining if expression of Rps15 candidate gene in plants increases resistance to Phytophthora sojae.

Single copy TO transgenic events will be selected based on the PCR analysis with primers specific to the cloning vector. Final confirmation of the Rps15 gene expression in plants for resistance to Phytophthora sojae will be done in the homozygous T2 plants for the Rps15 candidate gene. T2 plants homozygous for the Rps15 candidate gene will be expected to increase resistance to Phytophthora sojae isolates as compared to plants not carrying the Rps15 gene. These results would confirm the Rps15 gene.

### Example 7

This example demonstrates the identification of the Rps14 sequence.

PI 340029 carries broad resistance to P. sojae, including P. sojae races 1, 2, 3, 4, 5, 6, 7, 8, and 9. To determine if PI 340029 carries resistance to other races of P. sojae hypocotyl inoculation, as described above, was performed. As shown in Table 16, the hypocotyl inoculation studies determined PI 340029 also shows resistance to race 13, race 17, race 25, and two other isolates (ISA19A-1, ISA71D-1) from Indiana soybean fields and four isolates (MIN12001.01.05, MIN12004.01.01, MIN12004.03.01 and MIN12005.07.02) from Minnesota whose pathotypes that do not match any known P. sojae race designations. These findings demonstrate that PI 340029 can be a new source for broad P. sojae resistance.

TABLE 16
Responses of Soybean Landrace PI 340029 to Different P. sojae Isolates

| | | No. of | No. of | No. of | Proportion | |
|---|---|---|---|---|---|---|
| P. sojae | Virulence | plants | plants | plants | of | |
| Isolate | Patholotype | examined | survived | killed | resistant | Phenotype |
| Race1 | 7 | 12 | 12 | 0 | 1.00 | Resistant |
| Race17 | 1b, 1d, 2, | 24 | 24 | 0 | 1.00 | Resistant |
| | 3a, 3b, 3c, 4, | | | | | |
| | 5, 6, 7, 8 | | | | | |
| Race25 | 1a, 1b, 1c, | 12 | 12 | 0 | 1.00 | Resistant |
| | 1k, 7 | | | | | |
| Race13 | 4, 6, 7 | 8 | 8 | 0 | 1.00 | Resistant |
| ISA 19A-1 | 1a, 1b, 1k, | 10 | 10 | 0 | 1.00 | Resistant |
| | 4, 6, 7 | | | | | |
| ISA 71D-1 | 1a, 1c, 1d, 7 | 7 | 7 | 0 | 1.00 | Resistant |
| MIN1 | NA | 10 | 10 | 0 | 1.00 | Resistant |
| 2001 Jan. 5 | | | | | | |
| MIN1 | NA | 9 | 9 | 0 | 1.00 | Resistant |
| 2004 Jan. 1 | | | | | | |
| MIN1 | NA | 10 | 10 | 0 | 1.00 | Resistant |
| 2004 Mar. 1 | | | | | | |
| MIN1 | NA | 10 | 10 | 0 | 1.00 | Resistant |
| 2005 Jul. 2 | | | | | | |
| *Race2 | 1a, 7 | — | — | — | — | Resistant |
| *Race3 | 1a, 2, 3a, 3c, | — | — | — | — | Resistant |
| | 4, 5, 6, 7 | | | | | |
| *Race4 | 1a, 1c, 5, 7 | | | | | Resistant |
| *Race5 | 1a, 1c, 2, 4, 6, 7 | | | | | Resistant |
| *Race6 | 1a, 1d, 3a, 6, 7 | | | | | Resistant |
| *Race7 | 1a, 2, 3a, 3c, | | | | | Resistant |
| | 4, 5, 6, 7 | | | | | |
| *Race8 | 1a, 1d, 6, 7 | | | | | Resistant |
| *Race9 | 1a, 6, 7 | | | | | Resistant |

*P. sojae isolates previously used to evaluate PI 340029

By crossing PI 340029 with the susceptible Williams cultivar, 167 F₂ individuals were produced from self-pollination of the F₁. 57 F2 plants were tested for resistance to P. sojae race 1, of the 57 F2 plants, 48 were identified as resistant and 9 were identified as susceptible (Table 17). The null hypothesis that resistance was carried by a single locus cannot be rejected (x²=2.58, p=0.11). In order to get more accurate phenotypes, F_{2:3} families were tested for resistance to P. sojae race 1, the isolate avirulent to most known Rps genes. After harvesting seeds from the remaining F₂ plants, 20 to 36 F3 seedlings were tested. Among 110 F_{2:3} families, the segregation ratio of R (homozygous resistant): Rs (segregating): S (homozygous susceptible) observed for response to P. sojae race 1 was 28:48:34, which fits the expected ratio for 1:2:1 (x²=3.4, p=0.30) (Table 17). This indicates that resistance for P. sojae race 1 in PI 340029 is carried by a single Rps locus.

TABLE 17
Segregation Ratios of Phenotypes in the Mapping Population

| | | | | χ² goodness | | |
|---|---|---|---|---|---|---|
| | | | | of fit test | | |
| Parental lines and | Observed numbers | | | Expected | | |
| mapping population | R* | Rs | S | ratio | χ² | p |
| PI 340029 | 12 | — | 0 | | | |
| Williams | 0 | — | 12 | | | |
| (PI 340029 × Williams) | 48 | — | 9 | 3:1 | 2.58 | 0.11 |
| F₂ plants | | | | | | |
| (PI 340029 × Williams) | 28 | 48 | 34 | 1:2:1 | 3.4 | 0.30 |
| F_{2:3} families | | | | | | |

*R—homozygous resistant,
Rs—heterozygous resistant,
S—homozygous susceptible

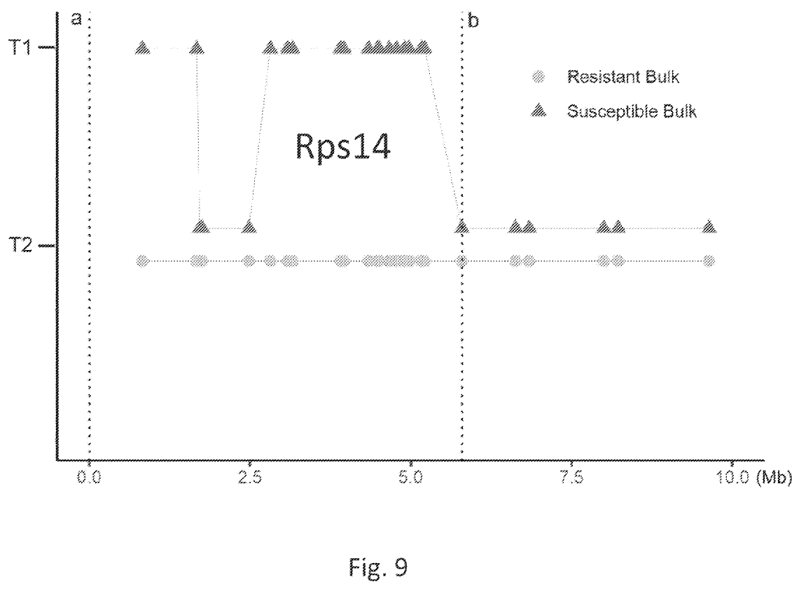

To detect the genetic region linked to the resistance gene in PI 340029, bulked segregant analysis was performed to initially map the Rps gene on a chromosomal segment. 10 completely susceptible F_{2:3} families and 10 completely resistant F_{2:3} families were selected to make up the susceptible and resistant bulks. SoySNP6K BeadChip consisting of 7039 SNPs was used to genotype both bulks and the two parental lines. There are 1983 SNPs markers in all 20 chromosomes showing polymorphisms between the two parental lines, which were effective and reliable for BSA analysis. After comparing the genotypes of the two bulks, a ˜5.8 Mb region was found at the beginning of chromosome 3, on which homozygous nucleotides that are the same as the susceptible parental Williams cultivar were found, while these nucleotides were found heterozygous from both parental lines in the resistance bulk (Table 18). Since no other regions on other chromosomes were found to be different between the two bulks, the chromosomal segment on chromosome 3 is considered closely linked with the rps locus according to the principle of BSA (FIG. 9). Since the region overlaps with some of previously defined Rps1 alleles/genes, the gene on this region was designated Rps14.

TABLE 18
Illustration of Genotypic Comparison Between the Resistant
and Susceptible Bulks at the SNP Sites Detected Between
the Two Parental Lines at the Beginning of Chromosome 3

| | | | | Resis- | Suscep- | |
|---|---|---|---|---|---|---|
| | Chromo- | Posi- | | tant | tible | Wil- |
| SNP ID | some | tion(bp) | PI340029 | Bulk | Bulk | liams |
| SNP178 | Gm03 | 829023 | TT | TG | GG | GG |
| SNP179 | Gm03 | 1671384 | AA | AC | CC | CC |
| SNP180 | Gm03 | 1718435 | GG | AG | AA | AA |
| SNP181 | Gm03 | 1758253 | CC | TC | TC | TT |
| SNP183 | Gm03 | 2818076 | CC | TC | TC | TT |
| SNP184 | Gm03 | 3087237 | AA | AG | GG | GG |
| SNP185 | Gm03 | 3907697 | GG | AG | AA | AA |
| SNP186 | Gm03 | 4487138 | CC | AC | AA | AA |
| SNP187 | Gm03 | 4509101 | AA | AG | GG | GG |
| SNP188 | Gm03 | 4665923 | CC | TC | TT | TT |
| SNP189 | Gm03 | 4782127 | CC | TC | TT | TT |
| SNP190 | Gm03 | 4903317 | GG | AG | AG | AA |
| SNP191 | Gm03 | 5165511 | AA | AC | CC | CC |
| SNP192 | Gm03 | 5217414 | CC | TC | TT | TT |
| SNP193 | Gm03 | 5796468 | AA | AG | AG | GG |
| SNP194 | Gm03 | 6631189 | GG | AG | AG | AA |
| SNP195 | Gm03 | 6844115 | CC | AC | AC | AA |
| SNP196 | Gm03 | 8003327 | TT | TC | TC | CC |
| SNP197 | Gm03 | 8228940 | AA | AG | AG | GG |
| SNP198 | Gm03 | 9641204 | AA | AC | AC | CC |

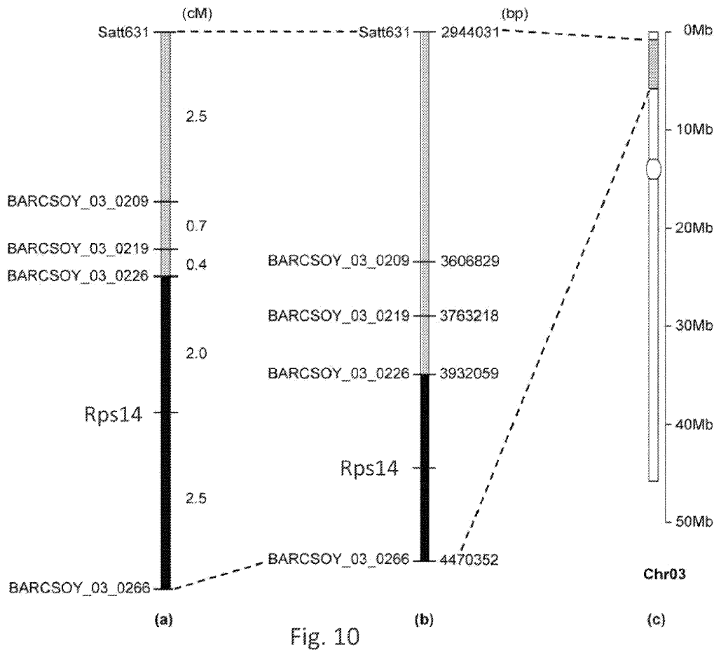

Based on the initial mapping results, a series of potential SSR markers located on the region mapped by BSA were screened and 5 markers, Satt631, BARCSOYSSR_03_0209, BARCSOYSSR_03_0219, BARCSOYSSR_03_0226 and BARCSOYSSR_03_0226, which showed clear polymorphisms between the two parental lines were identified (Table 19). These 5 markers were chosen to genotype all 110 F_{2:3} families. As expected, all markers observed a 1:2:1 segregation ratio, and 9, 4, 4, 2 and 6 recombinants between each of these five markers and the Rps14 locus were defined, respectively (Table 20). Among these 5 markers, Rps14 is more closely linked to BARCSOYSSR_03_0226 compared with the other four markers. A linkage map was constructed with these five markers and all the five markers were in good agreement with their order annotated on the Williams 82 reference genome (FIG. 10). The Rps14 locus was narrowed to a 4.5 cM region flanked by BARCSOYSSR_03_0226 and BARCSOYSSR_03_0266.

TABLE 19
Polymorphic Insertion/Deletion and SSR Markers Identified Between
PI340029 and Williams

| SSR Marker | Ch | Forward primer | Reverse primer |
|---|---|---|---|
| Satt631 | 03 | SEQ ID NO: 104 | SEQ ID NO: 105 |
| BARCSOYSSR_03_0209 | 03 | SEQ ID NO: 106 | SEQ ID NO: 107 |
| BARCSOYSSR_03_0219 | 03 | SEQ ID NO: 108 | SEQ ID NO: 109 |
| BARCSOYSSR_03_0226 | 03 | SEQ ID NO: 110 | SEQ ID NO: 111 |
| BARCSOYSSR_03_0229 | 03 | SEQ ID NO: 112 | SEQ ID NO: 113 |
| BARCSOYSSR_03_0266 | 03 | SEQ ID NO: 114 | SEQ ID NO: 115 |
| InDel3971 | 03 | SEQ ID NO: 116 | SEQ ID NO: 117 |
| InDel4033 | 03 | SEQ ID NO: 118 | SEQ ID NO: 119 |
| InDel4263 | 03 | SEQ ID NO: 120 | SEQ ID NO: 121 |
| InDel4330 | 03 | SEQ ID NO: 122 | SEQ ID NO: 123 |

TABLE 20
Chi-Square (X²) Goodness of Fit Test for the Markers in the F₂
Population Deduced Based on the F_{2:3} Progenies Derived From Pi
594592 × Williams

| | Observed numberª | | | χ² goodness of fit test | |
|---|---|---|---|---|---|
| Marker | a | h | b | χ²_{1:2:1} | p |
| Satt631 | 25 | 50 | 34 | 2.23 | 0.33 |
| BARCSOYSSR_03_0209 | 25 | 50 | 35 | 2.73 | 0.26 |
| BARCSOYSSR_03_0219 | 25 | 50 | 35 | 2.73 | 0.26 |
| BARCSOYSSR_03_0226 | 25 | 50 | 35 | 2.73 | 0.26 |
| BARCSOYSSR_03_0266 | 30 | 46 | 34 | 3.24 | 0.20 |

^{a}a means homozygous for the marker allele from the resistant PI 594592; b means homozygous for the marker allele from the susceptible Williams; h means heterozygous for the marker alleles from both parents

In order to narrow the mapping region of Rps14, two SSR markers, BARCSOYSSR_03_0219 and BARCSOYSSR_03_0266, were selected to genotype F₃ plants and 21 recombinants were identified. Subsequently, the F_{3: 4} families derived from these recombinants were tested with P. sojae race 1. One additional SSR polymorphism marker and four InDel markers developed from comparison between re-sequencing data of two parental lines to genotype these recombinants by pooling of F₄ seedlings from F_{3: 4} families was also used. Four recombinants (205-1, 83-3, 161-1, 174-2) defined the Rps14 locus to downstream of marker InDe14033, while 3 recombinants (59-8, 12-2, 152-5) defined the Rps14 locus to upstream of marker InDel4263. For the remaining 16 recombinants, genotypes and phenotypes were all consistent with the seven key recombinants. Therefore, the Rps14 was further mapped to the region flanked by InDel4033 and InDel4263, which defined a ˜229 kb region based on the Williams 82 soybean reference genome.

Previously, RpsUN1 was fine mapped to a ˜151 kb region defined by BARCSOYSSR_03_0233 and BARCSOYSSR_03_0246 (Li et al. 2016). The Rps14 region is ˜137 kb overlapped with fine mapped RpsUN1 region based on Williams 82 reference genome. There are 7 genes in that region including three predicted R genes with NBS_LRR domains (Table 21).

TABLE 21
Gene Annotation in the Mapped Region According
to the Soybean Reference Genome (Wm82.a2.v1)

| Genes | Annotation |
|---|---|
| Glyma.03g034500 | NB-ARC domain-containing disease resistance |
| | protein |
| Glyma.03g034600 | AAA-type ATPase family protein |
| Glyma.03g034700 | zinc ion binding; nucleic acid binding |
| Glyma.03g034800 | NB-ARC domain-containing disease resistance |
| | protein |
| Glyma.03g034900 | LRR and NB-ARC domains-containing disease |
| | resistance protein |
| Glyma.03g035000 | Nucleic acid-binding, OB-fold-like protein |
| Glyma.03g035100 | PIF1-like helicase |

RNA-seq revealed that only three genes expressed in the P. sojae infection process in the resistance donor line PI 340029, namely Glyma.03g034500, Glyma.03g034800 and Glyma.03g034900 according to reference genome Wms82.v2. al. All these three genes were annotated to encode NBS_LRR proteins. In the susceptible donor line Williams, these three genes show no or extremely low levels of expression in the process of P. sojae infection. Therefore, these three genes are all candidate genes for Rps14.

Previously, Rps1 (Rps1a, Rps1b, Rps1c, Rps1d, Rps1k), RpsUN1, Rps7 and Rps9 were also mapped to short arm of chromosome 3 and may overlap or be adjacent to the Rps14 region. To determine whether the Rps14 loci is distinct from these Rps loci, 204 accessions showing resistance to P. sojae according to USDA germplasm collection, 9 ancestral lines for Rps genes on chromosome 3 and the Rps14 donor line PI 340029 were selected for haplotype analysis. SNP genotypic data on the 540 kb region defined by SSR marker BARCSOYSSR_03_0226 and BARCSOYSSR_03_0266 were extracted. The generation of SNP data was previously described (Li et al., 2016), and a total of 31 SNPs in the defined region were called from these 213 different soybean accessions. Based on the topology of the neighbor-joining tree, the donor line of Rps14, PI 340029, belongs to a distinct branch compared with ancestral lines for other known Rps genes on chromosome 3.

By genotyping with two flanking markers (InDel4033 and InDel4263), eight homozygous resistance families were tested with eight homozygous susceptible families as control. Rps14 was resistant to all 13 P. sojae isolates, with all homozygous susceptible families and Williams showing intolerance to the disease (Table 22).

TABLE 22
Marker Assisted Resistance Spectrum Analysis of Rps14 to Isolates of P. sojae

| P. sojae | F_{3:4} families selected^{a} | | Parental lines | | Rps genes on Chr03 | | | | | |
|---|---|---|---|---|---|---|---|---|---|---|
| Isolate | Rps14 | Rps14 | PI 340029 | Williams | 1-a | 1-b | 1-c | 1-d | 1-k | UN1^{c} |
| Race1 | R^{b} | S | R | S | R | R | R | R | R | R |
| Race3 | R | S | R | S | S | R | R | R | R | I |
| Race4 | R | S | R | S | S | R | S | S | R | S |
| Race7 | R | S | R | S | S | R | R | R | R | R |
| Race13 | R | S | R | S | R | R | R | I | R | R |
| Race17 | R | S | R | S | S | S | R | S | R | R |
| Race25 | R | S | R | S | S | S | S | R | S | S |
| ISA19A-1 | R | S | R | S | S | S | R | S | S | R |
| ISA71D-1 | R | S | R | S | S | I | S | S | S | S |
| MIN1 | R | S | R | S | R | R | S | S | R | — |
| 2001 Jan. 5 | | | | | | | | | | |
| MIN1 | R | S | R | S | S | S | S | I | S | — |
| 2004 Jan. 1 | | | | | | | | | | |
| MIN1 | R | S | R | S | S | S | S | S | S | — |
| 2004 Mar. 1 | | | | | | | | | | |
| MIN1 | R | S | R | S | S | S | R | S | S | — |
| 2005 Jul. 2 | | | | | | | | | | |

^{a}InDel4033 and InDel4261 are two molecular markers used for selections. The resistance pattern of Rps14 against each P. sojae isolate was scored by the proportion of resistance progenies in 8 selected homozygous resistant F_{2:3} families. The reaction of Rps14 was evaluated from 8 homozygous susceptible F_{2:3} families.
^{b}A family was recorded as resistant if >75% of seedlings survived after inoculation, susceptible if <25% of seedlings survived, and intermediate resistant if the proportion of resistant seedlings was between 25% and 75%.
^{c}Phenotypes is based on Lin et al. 2013.

Rps14 was initially mapped to Rps1 region also harboring Rps1a, Rps1b, Rps1c, Rps1d, Rps1k and RpsUN1. However, none of these Rps1 genes showed similar resistance pattern as Rps14. The resistance pattern combined with the haplotype analysis demonstrate that Rps14 is likely a novel gene rather than a novel Rps1 allele.

### Example 8

This example demonstrates expression of the Rps14 in plants to increase resistance to Phytophthora sojae.

The Rps14 genomic interval will be further fine mapped using a large F₃ mapping population derived from a cross between PI 340029 and the susceptible Williams cultivar. The mapping population will be genotyped with molecular markers to identify recombinant lines. The recombinants will be phenotyped with Phytophthora sojae isolate Race 1. Based on the fine mapping the Rps14 QTL region will be narrowed down to a smaller genomic interval comprising candidate genes for further confirmation. For identifying candidate genes for Rps14, the whole genome sequence of PI 340029 will be generated using the PacBio sequencing platform. Contig harboring the flanking markers for the Rps14 genomic interval will be used as a reference sequence for comparing to the sequence data from the susceptible lines in order to characterize the genes and identify candidate genes based on the NBS type R-genes. The NBS type R-genes will be further analyzed for their gene structure to determine if it is a complete NBS type gene or truncated. Gene expression analysis will also be done to determine the expression profile of the NBS type R-genes in the recombinants. Together, with these analyses candidate genes will be expected to be identified for Rps14. Constructs comprising the candidate genes will be generated and transformed into soybean lines that do not carry the Rps14 candidate gene to confirm the Rps14 candidate gene and determining if expression of Rps14 candidate gene in plants increases resistance to Phytophthora sojae.

Single copy TO transgenic events will be selected based on the PCR analysis with primers specific to the cloning vector. Final confirmation of the Rps14 gene expression in plants for resistance to Phytophthora sojae will be done in the homozygous T2 plants for the Rps14 candidate gene. T2 plants homozygous for the Rps14 candidate gene will be expected to increase resistance to Phytophthora sojae isolates as compared to plants not carrying the Rps14 gene. These results would confirm the Rps14 gene.

All publications and patent applications in this specification are indicative of the level of ordinary skill in the art to which this invention pertains. All publications and patent applications are herein incorporated by reference to the same extent as if each individual publication or patent application was specifically and individually indicated by reference. Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting.

Terms used in the claims and specification are defined as set forth below unless otherwise specified. It must be noted that, as used in the specification and the appended claims, the singular forms “a,” “an” and “the” include plural referents unless the context clearly dictates otherwise.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

What is claimed is:

1. A method for identifying a soybean plant that displays increased resistance to Phytophthora, the method comprising detecting in a soybean plant or seed thereof at least one allele of a marker locus associated with Rps2b, Rps15, or Rps14.

2. The method of claim 1, wherein:
a. the marker locus is located within a chromosomal interval comprising and flanked by Satt431 and BARCSOY_16_1322;
b. the marker locus is located within a chromosomal interval comprising and flanked by InDe13437 and InDel3701; and
c. the marker locus is located within a chromosomal interval comprising and flanked by Satt631 and BARCSOY_03_0266.

3. The method of claim 2 wherein the at least one allele of the marker locus is detected by
i) contacting a sample of genomic DNA isolated from said soybean plant with a probe to the marker loci and screening for specific binding of the probe; or
ii) contacting a sample of genomic DNA isolated from said soybean plant with a pair of PCR primers, conducting PCR amplification and detecting the formation of an amplicon.

4. The method of claim 2, further comprising:
a. obtaining a first soybean plant comprising the at least one allele of the marker locus;
b. crossing the first soybean plant to a second soybean plant;
c. evaluating the progeny for the at least one allele; and
d. selecting progeny plants that possess the at least one allele thereby selecting plants with increased Phytophthora resistance as compared to a control plant lacking the at least one allele.

5. The method of claim 4, wherein the second soybean plant is susceptible to the at least one race of Phytophthora.

6. The method of claim 5, wherein the at least one race of Phytophthora is at least one of Race1, Race 2, Race3, Race4, Race5, Race6, Race7, Race8, Race9, Race13, Race17, Race25, or Race 31.


SEQUENCE LISTING (123 sequences)

SEQ ID NO: 1 - DNA, 14034 bp, Glycine max
  source 1..14034: mol_type = genomic DNA
atggacgctgtgtcatccgcactactagagccagtaactaattctgtgttggatctgcttaaaaagcaagtggattacatccgttacaggcgaaactttgatgaactagacgagtgtgttaagcagcttaaacataaaaaggagatagtagatcatcaatgtgaggaagctgtcaaaaatggacacgaaattgaaggtaaggttagagaatggttagggaaagtgggtaaatttgagacagaagtggagaagtattggaacgatgatggccacaaaaagacacggttttccaactatttatttccttactttaggcatagactaggcagactagcaaagaagatggcagttgagggtaaaaagataaccgatgattgcccaaagtctgatgaaattgcctatagggtatacgtaacatctaattatgccattttgtctaataatgaccttatggattttggttctagaaaatccataatggaacaaataatggcaacacttgttgaagatcccactgtgaaaatgattggagtgtatggacgaagtggggtgggtaagagcactttaatcaaagcaattgctaaaattgctcgagacaagaagttgtttaatgtggtggctttttcagaaataacagacaaccccaatctaaaacaagtccaggaagatattgcttaccctttgggattgaaattggaaggagaaggtgagaatgtaagagctgatcatctacgaaggaggttaaagaaagagaaagagaacacccttataatcttggatgacctttgggacagattagacttgaataggttgggaattccacttgatggtgatgttgatgataaacagggtcccaaagggccgacaaaagaaaaatctcttgctgattataagggttgcaaaattttgctaacttcaaggaaacgaaatgtattaacggataaaatggaagttaaattaactttctgtgtagaggaattagatgaaaaagatgctctgaagttgtttcggaaggaggctgcaatacaaggtgaaatgtccaagtctaaaaaagaaattgttaagaagtattgtgctgggttacctatggcaatagttacagttggaagggcattaagagacaagagcgactcagagtgggaaaaacttaaaaaccaagaactggtgggagttcagaatccgatggagatttctgtaaaaatgagttatgaccatctagaaaatgaggagctcaagtccattttctttctttgtgctcaaatgggtcatcaacccctaattatggacttggtgaagtattgctttggtttgggaatacttgaaggggtctactcgcttggggaagctcgggacagaatatctacatcaatcaaaaagctgaaagactcaggtttggtgttggatggaagttctagtattcatttcaatatgcacgatctggttcgagatgctgctttatctatagcacagaacgagcaaaatgtatttactttgagaaatgggaaacttaatgattggcctgaactcaagaggtgcacttctatttctatatgcaatagtgatatcattgatgagcttcctaatgttatgaactgtcctcaacttaaatttttccaaattgacaatgatgatccatctttaaaaatacctgagagtttttttaagagaatgaaaaaactcagagtgttaatattgactggctttcatctatcaagcttaccatcatcaattaagtgcctatcagacctcagattgctttgtttggagcgatgcactttagatcacaacttatccatcatagggaagctgaaaaaattaagaattctcagcttttctggatctcgaattgaaaatttgccagctgagttgaaggacttggataaactacaattactagacatcagcaattgttcaatagtcaccatgattccacctaatcttatatcaaggttgacttcgttggaagagctgtatgtaagaaagtgtttcatggaagtgtcggaggaaggagagagaaaccaaagtcaaaattcatttatttctgaactaaagcatttgcatcaattgcaagtggtggacttaagcattccatgtgctgaattttttgcaaaggaattgttctttgacaacttaagtgattacaagattgagattgggaacttcaaaactctttcagctggagatttcagaatgcctaataagtatgaaaatttcaaatctttggcattggagctgaaggatgacactgacaatattcactctcagacaggaataaagttgttgtttgaaacagttgaaaatttgttgttgggagagctgaatggtgttcaagatgttgttaatgagttgaatttgaatggatttccacatctgaaacacttttccatcgtaaacaaccctagcatcaaatatatcatcaactcaaaggatttgttttatcctcaggatgtttttcccaagttggaatctctatgcctctacaaactaaaagagatagagatgatatactttagttcaggtacagagatgatatgctttagtccatttacagattgctcattcaccaaattaaaaaccatcaaggtcgagaagtgtgatcaattgaagaatcttttctccttttgcatggttaaattgcttgcgagtcttgaaacaattggtgtttccaattgtggttctttagaggagatcattaaaataccagacaattctgataagattgagtttcttaagttgatgtctttgtcacttgaatcattatcatcattcactagtttttataccacagtagaggggtcttctacaaacagagatcagatacaaattactgttatgactcctcctctttttggtgaactggtatgatagtgcatatttttcctttaaaaattttaattagtctgtgactacttatttttcagttgcaaatatgttaattgtgtatgtgcttgcaggttgaaataccaaacttagagaacttgaatttaatctcaatgaacaagatccagaagatatggagcgaccagcccccgtcaaacttctgctttcagaacttaataaaattagttgtgaaagattgtcaaaatttgagatatttgtgttcattgtccgtggccagcagtttgaggaaactgaaaggcctctttgtaagcaactgtaaaatgatggagaagatttttagcacagaaggaaatagtgcagacaaggtgtgtataggtgtaaataagtaaatttttatagttgactgaatgttatgcatttctgtttagtttcattaccaaataatggcttgttttcatcgaagtgcaggtttgcgtctttcctaagttggaggaaattcacctcgaccaaatggatgagttaacagacatatggcaagctgaagtgagtgctgattccttttctagtctcacttctgtgaacattcgtagttgtcataaactagacaaaatttttccgagtcacatggaaggatggtttgcgagtttgaacagcttgaaggtttctttttgtgagtcagtggaagtgatttttgaaatcaaagattctcagcaagtagatgcatctggtgggatagacacaaatttgcaggttgtttatgtaagtgaactcccaaagttggagcaggtgtggagcagggatccaggaggaattcttaacttcaaaaaactgcagagtatagagatggatgattgtgaaagactgaggaatgtatttccagcttctgtgggcaaagatgttccaaagcttgaatacatgtcggtcatagagtgtgatggaattgtggaaattgttgcctgtgaagatggatccgaaacaaacactgaacaattagtgtttcctgaactaaccgacatggaattatgttacctatcaagcatccagcatttctacagggggagacatcctatagagtgtccaaaattgaagaagttgtcagtagggaaatgtaacgagaagctaaaaacattcggaaccggagaaaggagcaatgaagaagatgaagcagttatgtcagctgaaaaggtaagtcatatagtgagacataaatggagagtgatattgtaagatgtgtggcatacttaaaaaagatatagccctgctaaatataatatggattattaatttaacgtctctcgtcccctaatccctctaaatccaaattcctcattgtgccttaaaattttatttagaatttagaattgaagggaatgtttgattaattattataatttaagagcattttttagttttgatgggacccgagagtaaccaatgtgcaggcaaggcgtaaaataatggaaaggggcgctccatcagggataagcttccttcttcatagtttactatttacttttattttatttttctgtagatattccccaacttggagtatttggatattcactttgacgaagcacagaagtggttattgagcaacactgtgaagcatcgaatgcaccgtttaaaaaagcttaggttaagcgaagttaatgatggtgaacgtctctgtcaaattctgtacagaatgccaaatctagaaaagttatacttgccgatggctaaacatttgcttaaagagtcgtcggagtcccgtttgggaaccctattacagctgaaggaattggatttgtggaggtcggagataaaggatataggatttgaacgagaaccagttctacagagactagagcttttgagcttatttaagtgccataaattgaggaatttgggtcctccctcggtatcattggcttacttgacaaatttgaaagtagagtattgttatggattaaggaatttaatggcatcctcaacggcaaaaagcttggttcaacttaagtccatgaagataagaaaatgttgtaaattagaggaaatagtaagcgatgagggaaatgaagaagaagagcaaatagtgtttggcaaattgattactatagaacttgaggggctaaaaaagctgaaaagtttttgcagttccaagaactatgaattcaaattcccgtcattggaaggattgattgtgagaaaatgcccaatgatgcatacattcacggagggtgacgcaagagcaccaaagttagaaaacacagttactgctaaagaagaaggaaaagaggaagccaaatggcagtgggaaggagacttgaattccaccatacaaaaaggtttcaacaaggtacttattcatttatttatgatttaaatatatttttatttatcttgttagtatttttttattcttccttttttatttttgttcttcaaagttattatttttgttaattttagtctttttaggttatttcattcatttttaatacgttaattttttttaaaattttagtctctttaatattttaaaatattcatttataatcctccgcacgaaattaaaattaaaaaatataaacttataaaataaaaaatgagtaaaacatattacggagatcaaaattaaaaaagcataaactttcaacgactaaaattaaaaaaaaaaaacttaccagagacaaaaattaaaaaatattaacttacatttatcaaatacatatttaaacttatatttattacaaattctcctagttttattaacttttcttctttccttacaaaaacaaacttttcttctttcatgagataaaaaaaattacttgccatgaatggaaattataaatccttgtttttcccataaaaagccttaaccagcaagtatgttaaatttcttaagatataaatatgttagctcaaaaaaccagcatttatataaatttattaatcatttttgttttaaaaaaattcattcataaaatttaaaataatacgaatgtgactgcttcatgtagccagcgtagtcataaagaaaacattccctcatcttacaacttttttttttaattataataaataaacttcaattcatatttgattatcatccattaattttttgttaacatttttagtccttttagaaatttttgctcattattaatcctattgtttatgttatatttaagaaaaaaaaacgactaaaaatggatcgatgaacacatttttagagggattaaaactttaaagaaagattgaaatttgtaaaaaaattgagggggaaaaattagaatataaaaaaatgaggagttaaaaacttaattacatttgactttacatatataaaataacatataaacgtgataataaattaacaaacttatccatataatttgtgactatttatatatctataaaataaaagtcacatgagacgttattataacttacaaggttgattttgtagttggaaaaaagaatttgaagggaaaaggatgaaagaggttttaagttacgatactttcactaatattttaataaaaaactaaaactaacaagtaatattaattgatgaaaaaaatgtcttttatacatacggaaaaataaggatgaaatgtatttttttttctttttttgttaaaatataaaatatcgtgaaaataaaatgacattattacacctaagagtaaatgataaaatatttcttaatcgaataatccataatttcatcattgataaaaataaattatcatgttatttacttatatattttttgtttaatcttttgtgcagctatccatatttagtattttagcacatgaatggcagaaagtaaatatttatttatacaacaagattgtaatttataataaataaatattttaagtatataaattatattataattaaattttctaatatataatttttaacatataaattacattttaaatttatgaaaaataatttatgttaggatgtattattattttaaataattgatgttttcttttaaaaatatttaaattaagttagaaacaaggatgccgtcaagttttcttacagcctacatttatcgtgatgaaggaaggaaggtgttttaatggtcccctgatgatgaaacttggaatgtaattttataataaacaacggacataattgctccctccatattaatatctgtttgccacacaactcagtcccatcattatttggcgttgacttcttcatcttataccaagatagcaagagtccatggattcattttttattattaattattaattatataaaaatttatttatatttattatccatttgactattgagttttaagatattaggtttttttcattaaaatataataaatatatttttaattaatatgataatctcaaagtaaaaaaattattatatttattattaagaatttttcattgaaagtttaaaatattgttaacaactaaaaattttcacttcaattaatttaattaatttaataatgtcatccacatgtgaattatttatatatataatttaataaataagattaattatttttcttcaataaagacggttatggatacattaatctgtctccattattattatcttatttcattataatcttatgattaaaaataaaatagattaaaattataaaagaattgattagtattattattataatttctatcatatagcaagtgtttaattttaatccagatctatcaagttaacaatttaataataaataaataataatatttttatcaaaattaataatatgatggattagatctttaacatacatatttaattctagcaatacacacatatatatatatatatatatatatatatatatatatatatatatatatatatatatataattttacatttttacttatggtttatctaaaaataataaaataaatctatgatcttatagtttatattataaatagctttctttattaattaataatattttttataatatagagctattttttcatttgttaaacccatgtttgtttgaatttttattcaaatttttctatgatttttttctacaaattgttataattttgatagagaaaaatatgattatcttctttaagttcattaaaaaatccatagatttttttccctgtatcctcacacaaatattttgtatataaacaaacaaactaaatgtttcaggttcattttttcacacaaaaaaatggaaatgaaatttttaatttttcagacaaaagtaaaaaaaaaaattatactatataaaaaatattttggttcctccaatattttttttctcaaagttccgccactgatttcatgcgtctctatgtctaatatcaagttcctctctttttctttgtctttttattaactaaattcagtattaagacagtaactgatttatgactaatctatctatttgttataatgtaatcgccagctatgaaactgacatattagccttttgtatgcaatatcctattttgcatgcacagcttttggagtctgcaagtactgaatcatctcttagtctcatagatagcccactacaagtgatatggcttgactcacggcggatcccaaagtcgtgcttcagtaacttgacggaattgactgtgcacggatgccaatttttaacagatgttgtcatacccttctatttacttcctttcttaactaatttgcaacaattacaagtctcggactgtcgttctgtgaaaagcatatttgacgtgaaaacagctatgggattgggagcagcagccttccctagacctctccctttttccctcaagaaattgactttagagtggctgccaaaactggagaatgtctggaatgaagatcctcatggaattctaaccatgcaacttctacaacatgtaaaggttaaaaattgtaaatgccttacaagtgtgtttccggcatcattagccaaagatcttgaaaaactagttgtcaaagactgtgagggattgatagaaattgttgcagaggataatgcagatccaagagaagcaaatctggagcttacgttcccttgtccctgtgtgagctcattgaaactacaaggtttgcccaagttcaagtatttttactactgctcactgcagtgtgacatgttccagacacctaccaaggatgaaatggtactattgtgttactactttgtccttactgcataatctcatgcctgcaaattaaatataattgcactacttcatttttattgttcaacatgagtgatatgaacattctcttcaaattaaaatgtgagaatttcacgtacattgctatatatatatgccgtgttctatcttctatacctattaactcccatttttcatgttttacataaataagtaaaagaatacacttaagggtacatgttgcattcatgacacaacaatgacattttaggtttgagctttattcttattaatacagacaattacatagaatcttacacatgccattttactttatcaaatacaatgtatggatggttttcaaatctttataaacaatctccctaccatataaaggatcgaatttttcatcattttcttctccgttggaaccactactacctcccttaaggtctcctttatcaccttttccatctaaaattttaaactacaaataaataaatttgtttaggcacaaaccgtatcaaaatcccttccaccttattaatagctagctagccaggttcatctcttttataacctcactaacctgcttttgcttggttatcggtttctcttggtagcctacatccaacttacagtgcctgtcactcggtgaaaaaggactggagatgatcaagcgtgcagaatttcagagaaacttcttacacaagttacaagttcttactctgtgctttcatattgggtcgaatgtatttccatatgaaattctacaactggcgcccaatatagagaagcttgtggtgtgtgatggttccttcaaggagattttctgctttgatagtcttaatgtggatgaggctggactcctattacagctcaaagtcttatgcttggagtcccttccagagcttgtttccattgggttagagaactcttggattcagcccttactgggaaatctagaaaccttggaagtaataggttgttctagtttaaaagacttggtaccatctacagtgtctttttccaatctgacatatttggaagtagaaagatgccattgcctgctatatttgttcacatcctccacagcaagaagtttgggtcaactcaaaagaatggagataaaatggtgtggttcaattgaagaggtagtagtctctaaggagggggatgaatcacatgaggaggagattatatttccgcagctcaattgtttgaaacttgaatatttatcaaagctgagaagcttctataaaggaagtttattaagtttcccatcattggaggaattgtcagtaatccgttgcgagtggatggaaacattatgtccaggtacccttaaagcagacaagttggttcaagttcaacttaaggagagttattcttggaggcactcagatcctatcaaattggaaaatgacctgaactctaccatgcgggaggcattttggaaaaaggtatgttttcaattattttgattaaatatgattgatattggtgtatgttgttcttataatgcaaaatatacaccgttaggttgattagattcatcttcaaattaacttgggcctcatccaatttatcttggattttcagcgtcgtccaatttttcggaaaaccaaaaaactcacagaaacaacatttatgtgccaatccaatttggctaatattaattttttacgttaaactcataatatttaaataatttaatttattttctatgtactttatcaatgctacttaacttttatatatattaataatataaaatgaatccaaactctttcaagtcattgtaaaaatgatttgaaaattatttaatttttattttatttatattaatcctattaaataaaagaaactatcaatcttttgtaatccaatcttatatacatattcaaaatatttatcttttagcaattttaactatataaaaaaattatcttctaggattaagatactatataattatatttaaaatctagattaggtagttaaaatgttagttgttcatttttttttaattttctttaactttttgttatttcttcatttatacataaaaataaattaaataaatattaaaaaaattagagtgacttgaagaagtttagattcactgtatattattattattattaattactaatacaaataaaagttaagtagtataagtaaggtatataaaaaataattatttaaatattagtgagtttagcttactaaaaaattaagagattctttttttagtataaggtaagaggttaatataagtcaaataattttgtttgtcaacttaattgtatgacgtgactgcaagctcaatctaatggtatatattgtgttggagagaaaagtatgttcaagagcatctggatcttatattttaacggagattaaatgtgttgtatttattagtattcattgcatatatgaattttttataagaaatggtcattaaatttgtgcgattttcttataaataactcaccagttgagactaataaatatgctaaatttttcttaatttttctaagtaagataaaaaaatactcgatcgactatctagctttgatcatgcgtatgttttgtcaaatgatgaattttgaattttgaaaaataacctactctttcttatacaaatatctagaaacttttttatgagtgcaacaaagacatataaaaaatattctggaaaatactttcattgtgtgttgataaatttagaatgaatgtaccagaatcaactagttgaaaactattatctcaggcgcattcatccagaaatcaactcaaggaggagctgattttttttcctttacaaattatttaaataattttgaaggcagattttgattagagagcatccacgattaacggaaatctggactataatcagaaatttgttacttttttaatttattgttttaaaaataattacactttaaaaattgagagcacattagttatttggtgcgattggggatgcatttcttcctacagaataatactactctttttttttattcttttacgtttgcagttatggaagtctgcagatacggaattcagtattgacctcaaagatagcccagtacaagagatatggcttaggcttcactcactgcatatccccccacacttctgcttccctaagttacacaccttgattgtggacggctgccattttttatcagatgcggtcttacccttctctttacttcctttattacctaatttgaaaacattggaagttcgaaactgtgattttgtgaaaatcatatttgatgtgacaactatgggaccactcccttttgccctcaagacattggcactgtgtgatctgccaaatctggagaatgtttggaattcaaatgttgagcttacgttcccccaagtcaagtcattggcactctgtgatctgccaaagttaaagtatgacatcttgaagccatttacacatctagaaccacatactctaaatcaagtctgtattcaaaaggtatcattactacttctatatatatgaactaaattgccatcattagtgttctttactacttctatatatatgaactaaattgccatcattagtgttctttactacttctatatatatgaacaaattgccatcctgttaaaattaaaaagttgtgaacctttcccttatacagttatatatgctatgttgagtcttctatccatcttctacgcatattggcttccatttttctattttttatttatctacataagtcagataatacggctacagctactattcaatattgatcatgatgaatttataatggaacatgttgcattcatgatgtgaaacagcatttgaggtgcgatccttattctcattattttcttctacaaccttactaataatctcatctatcacttttcccatcttaaaatattaaagtcgacatacggtgtgaatatcctttctccaccttattctttttatggtatccttttccaccttataaatagctagcctagttcgtctttttttcttttttggtttttaagtaaactgatttcttttaaattgacttgtttgcacctatttaattgaattggttaatttgtcattaattattggacttattttagcgtgaaagttcattatttcaattatataaaaagagctggatctataacttccctaacctgctttttcttcattgttggtttctcttggtagcttacacccaacatagagcacctgacactcggtgaacatgaactcaacatgattttgagtggagaattccagggaaaccacttaaacgagttaaaagtgcttgctctgttctttcatattgaatccgatgtatttctacaacgggtgcccaatatagagaagcttgaggtgcgtgatggttccttcaaagagattttctgctttgatagccttaatgtggatgaggatggattggtttcacagctgaaagtgatatgcccggactcccttccagagcttgtttccattgggtcagagaactctgggattgtgccctttctcagaaatctagaaacattgcaagtaatcagctgtttcagttcaataaatctggtaccatgcacagtgtctttttccaatctgacatatttgaaagtagaaagttgcaagagtctgctatatttgttcacatcctcaacagcaagaagtttgggtcaactcaaaacaatggagataagttggtgtaattcaattgaagagatagtgtcttcaacagaggaaggggatgaatcagatgagaatgagataatatttcagcagctcaattgtttgaaacttgaatttttatttaagctgagaaggttctacaaagggagtttaagtttcccgtccttggaggaattcacagtatggcgttgcgagaggatggaaagtttgtgtgcaggtacagtcaaaacagacaagctgttacaagtgaatactaattggggcggagatgttatcccattggaaactgatctgaactctgccatgcaaaaccgatag

SEQ ID NO: 2 - protein, 2463 aa, Glycine max
MDAVSSALLEPVTNSVLDLLKKQVDYIRYRRNFDELDECVKQLKHKKEIVDHQCEEAVKNGHEIEGKVREWLGKVGKFETEVEKYWNDDGHKKTRFSNYLFPYFRHRLGRLAKKMAVEGKKITDDCPKSDEIAYRVYVTSNYAILSNNDLMDFGSRKSIMEQIMATLVEDPTVKMIGVYGRSGVGKSTLIKAIAKIARDKKLFNVVAFSEITDNPNLKQVQEDIAYPLGLKLEGEGENVRADHLRRRLKKEKENTLIILDDLWDRLDLNRLGIPLDGDVDDKQGPKGPTKEKSLADYKGCKILLTSRKRNVLTDKMEVKLTFCVEELDEKDALKLFRKEAAIQGEMSKSKKEIVKKYCAGLPMAIVTVGRALRDKSDSEWEKLKNQELVGVQNPMEISVKMSYDHLENEELKSIFFLCAQMGHQPLIMDLVKYCFGLGILEGVYSLGEARDRISTSIKKLKDSGLVLDGSSSIHFNMHDLVRDAALSIAQNEQNVFTLRNGKLNDWPELKRCTSISICNSDIIDELPNVMNCPQLKFFQIDNDDPSLKIPESFFKRMKKLRVLILTGFHLSSLPSSIKCLSDLRLLCLERCTLDHNLSIIGKLKKLRILSFSGSRIENLPAELKDLDKLQLLDISNCSIVTMIPPNLISRLTSLEELYVRKCFMEVSEEGERNQSQNSFISELKHLHQLQVVDLSIPCAEFFAKELFFDNLSDYKIEIGNFKTLSAGDFRMPNKYENFKSLALELKDDTDNIHSQTGIKLLFETVENLLLGELNGVQDVVNELNLNGFPHLKHFSIVNNPSIKYIINSKDLFYPQDVFPKLESLCLYKLKEIEMIYFSSGTEMICFSPFTDCSFTKLKTIKVEKCDQLKNLFSFCMVKLLASLETIGVSNCGSLEEIIKIPDNSDKIEFLKLMSLSLESLSSFTSFYTTVEGSSTNRDQIQITVMTPPLFGELVEIPNLENLNLISMNKIQKIWSDQPPSNFCFQNLIKLVVKDCQNLRYLCSLSVASSLRKLKGLFVSNCKMMEKIFSTEGNSADKVCVFPKLEEIHLDQMDELTDIWQAEVSADSFSSLTSVNIRSCHKLDKIFPSHMEGWFASLNSLKVSFCESVEVIFEIKDSQQVDASGGIDTNLQVVYVSELPKLEQVWSRDPGGILNFKKLQSIEMDDCERLRNVFPASVGKDVPKLEYMSVIECDGIVEIVACEDGSETNTEQLVFPELTDMELCYLSSIQHFYRGRHPIECPKLKKLSVGKCNEKLKTFGTGERSNEEDEAVMSAEKIFPNLEYLDIHFDEAQKWLLSNTVKHRMHRLKKLRLSEVNDGERLCQILYRMPNLEKLYLPMAKHLLKESSESRLGTLLQLKELDLWRSEIKDIGFEREPVLQRLELLSLFKCHKLRNLGPPSVSLAYLTNLKVEYCYGLRNLMASSTAKSLVQLKSMKIRKCCKLEEIVSDEGNEEEEQIVFGKLITIELEGLKKLKSFCSSKNYEFKFPSLEGLIVRKCPMMHTFTEGDARAPKLENTVTAKEEGKEEAKWQWEGDLNSTIQKGFNKLLESASTESSLSLIDSPLQVIWLDSRRIPKSCFSNLTELTVHGCQFLTDVVIPFYLLPFLTNLQQLQVSDCRSVKSIFDVKTAMGLGAAAFPRPLPFSLKKLTLEWLPKLENVWNEDPHGILTMQLLQHVKVKNCKCLTSVFPASLAKDLEKLVVKDCEGLIEIVAEDNADPREANLELTFPCPCVSSLKLQGLPKFKYFYYCSLQCDMFQTPTKDEMPTSNLQCLSLGEKGLEMIKRAEFQRNFLHKLQVLTLCFHIGSNVFPYEILQLAPNIEKLVVCDGSFKEIFCFDSLNVDEAGLLLQLKVLCLESLPELVSIGLENSWIQPLLGNLETLEVIGCSSLKDLVPSTVSFSNLTYLEVERCHCLLYLFTSSTARSLGQLKRMEIKWCGSIEEVVVSKEGDESHEEEIIFPQLNCLKLEYLSKLRSFYKGSLLSFPSLEELSVIRCEWMETLCPGTLKADKLVQVQLKESYSWRHSDPIKLENDLNSTMREAFWKKLWKSADTEFSIDLKDSPVQEIWLRLHSLHIPPHFCFPKLHTLIVDGCHFLSDAVLPFSLLPLLPNLKTLEVRNCDFVKIIFDVTTMGPLPFALKTLALCDLPNLENVWNSNVELTFPQVKSLALCDLPKLKYDILKPFTHLEPHTLNQVCIQKLTPNIEHLTLGEHELNMILSGEFQGNHLNELKVLALFFHIESDVFLQRVPNIEKLEVRDGSFKEIFCFDSLNVDEDGLVSQLKVICPDSLPELVSIGSENSGIVPFLRNLETLQVISCFSSINLVPCTVSFSNLTYLKVESCKSLLYLFTSSTARSLGQLKTMEISWCNSIEEIVSSTEEGDESDENEIIFQQLNCLKLEFLFKLRRFYKGSLSFPSLEEFTVWRCERMESLCAGTVKTDKLLQVNTNWGGDVIPLETDLNSAMQNR

SEQ ID NO: 3 - DNA, 2700 bp, Glycine max
  source 1..2700: mol_type = genomic DNA
atggctgcaaaaacacgttcccttgcatccatctatgatgtgttcctcagcttcagaggtttagacacacgccatggtttcactgacaatctctacaaagctcttgatgacaggggaatctacactttcattgatgatcaggagtttcccagaggagacgaaataacacctgcactttccaaggcaattcaagagtccaggattgctattactgtgctttctcaaaactatgcttcttcctcgttttgtttagatgaacttgtaaccatccttcactgcaagagtgaagggctgttggttataccggtcttttataaggtagatccttctgatgtcagacaccagaaaggtagttatggagaagcaatggctaagcatcagaagaggttcaaagctaagaaggagaagctgcagaaatggaggatggctttgaaacaagtagctgacttgtctggctatcatttggaagatgggtataaaaccatactaatatattttactttatggtttttattggattaggttttacttgtctattgatttaactagtaaaattcaaataggaaagaaatgttcttgttaaccttgacaattattgtacctatcgagacatggatgcaaggattttaggctgacttcatcagaactttttttgtttgtttgaaacagagatgcatatgaatacgaatttattgggagtattgttgaggaggtctgtaggaagattagtcgtgcttctttacatgctgcggattatccagttgatctagagtcacaagtgacaaaggtaatgaagcttttgtatcttggatcccatgatgttgtccacatcatagggatccatggaatgggcgggttaggaaaaacaacacttgctcgggcagtttataatttgattgctctccattttgatgaatcatgttttcttcaaaacgtgagagaagaatcaaataaacatgggttaaaacaccttcaaagcacccttctttcaaaattacttggtgaaaaggacatcaccttaacaagttggcaagaaggagcttcaatgatacaacataggctccgaagaaagaagattctcttgattttagatgatgttgacaaggacgagcaattaaaggctattgttggaagacctgattggtttggtcccggtagcagagtcatcattaccactcgggacaaacatctgctaaaatgtcacgaggttgaaataacttttgaggtgaatgatttgaattacaaagctgtaacaaaatgggaatctgctgttttatatttttataaaagaattcccagtgatgaaatccaagagatactaaaagtaagctttgatgctttggaggaagaacaaaagaatgtttttcttgatattgcttatttcttgaaagggtataaatggaaagaggttgatgatatactccgtgctctttatggtaactgcaagaagcatcatattggggagttggttgaaaaatctctcataaagcttaactgctatgatggtggtactgttgaaatgcacgacctgcttcaggacatgggtagagaaattgggcggcagagatcaccagaagagccctggaagtgcaagagattatggtcaccaaaagatataattcaagttttaaaacacaacacggtgagtgagctcatgaatagttgaattttttttgtctatctcatatttacatcatggttaactttttcttgataatttgtgaatttctctaagcgagtcaattgatatttcacacaaattgtattgtggttgattcgtgcctttttgcattagcagggaactagtaaaattgaaatcatatgtctggattgctccatatctgagaaagaagaaacagtggaatggaatgaaaacgccttcatgaagatggaaaaccttaaaatacttattattagaaatggcaaattttccaaagctcccagttattttccagaaggtttgagagtactggaatggcacagatatccttcaaattgtttaccatctaactttaatccgaacaaccttgttatatgcaacttacctgacagttccatgacgtcatttgagttccatggctcatcgaaggcatgtttaaagagtatattttcctcattgcatggctcatcgaaggcaagtttaaagagtatattttcctcattccatgaattaaatttattcatttgtttcttatttcttttcttttttctttttttttgcctttgcagaagctggggcatctaacagttttgaattttgactggtgcaaatttttaacccagatacctgatgtatctgatcttccaaatttgagggaactttcatttcgatggtgtgagagtttagttgcagttgatgagtcaattggttttctgaagaaacttaaaatattgaatgctgctggttgcaggaagcttacgagttttccgcctctcaacttgacctctcttgaaacacttgaactttctcactgttccagtcttgagtattttccagaaatattaggagagatggaaaacataaaggcacttcatttggaaggccttcccataaaagaattgccattttcatttcaaaatcttattggactccgtgagataaccctgaggaggtgtagaattgttcggttacgatgtagcttagccatgatgcccaatctgtttcgattccaaattagaaattgcaacagctggcaataa

SEQ ID NO: 4 - protein, 750 aa, Glycine max
MAAKTRSLASIYDVFLSFRGLDTRHGFTDNLYKALDDRGIYTFIDDQEFPRGDEITPALSKAIQESRIAITVLSQNYASSSFCLDELVTILHCKSEGLLVIPVFYKVDPSDVRHQKGSYGEAMAKHQKRFKAKKEKLQKWRMALKQVADLSGYHLEDGDAYEYEFIGSIVEEVCRKISRASLHAADYPVDLESQVTKVMKLLYLGSHDVVHIIGIHGMGGLGKTTLARAVYNLIALHFDESCFLQNVREESNKHGLKHLQSTLLSKLLGEKDITLTSWQEGASMIQHRLRRKKILLILDDVDKDEQLKAIVGRPDWFGPGSRVIITTRDKHLLKCHEVEITFEVNDLNYKAVTKWESAVLYFYKRIPSDEIQEILKVSFDALEEEQKNVFLDIAYFLKGYKWKEVDDILRALYGNCKKHHIGELVEKSLIKLNCYDGGTVEMHDLLQDMGREIGRQRSPEEPWKCKRLWSPKDIIQVLKHNTGTSKIEIICLDCSISEKEETVEWNENAFMKMENLKILIIRNGKFSKAPSYFPEGLRVLEWHRYPSNCLPSNFNPNNLVICNLPDSSMTSFEFHGSSKACLKSIFSSLHGSSKKLGHLTVLNFDWCKFLTQIPDVSDLPNLRELSFRWCESLVAVDESIGFLKKLKILNAAGCRKLTSFPPLNLTSLETLELSHCSSLEYFPEILGEMENIKALHLEGLPIKELPFSFQNLIGLREITLRRCRIVRLRCSLAMMPNLFRFQIRNCNSWQ

SEQ ID NO: 5 - DNA, 2661 bp, Glycine max
  source 1..2661: mol_type = genomic DNA
tgtcattgttaccatatgtcgtgttactctgcgtaataataaacattttgtatgctatctttttcatatttgttcaagtttgattctacactcccaccatatcaaccataatcatactaaacttttttcttctaataatggctgcaaaaacacgttcccttgcatccatctatgatgtgttcctcagcttcagaggtttagacacacgccatggtttcactgacaatctctacaaagctcttgatgacaggggaatctacactttcattgatgatcaggagtttcccagaggagacgaaataacacctgcactttccaaggcaattcaagagtccaggattgctattactgtgctttctcaaaactatgcttcttcctcgttttgtttagatgaacttgtaaccatccttcactgcaagagtgaagggctgttggttataccggtcttttataaggtagatccttctgatgtcagacaccagaaaggtagttatggagaagcaatggctaagcatcagaagaggttcaaagctaagaaggagaagctgcagaaatggaggatggctttgaaacaagtagctgacttgtctggctatcatttggaagatggagatgcatatgaatacgaatttattgggagtattgttgaggaggtctgtaggaagattagtcgtgcttctttacatgctgcggattatccagttgatctagagtcacaagtgacaaaggtaatgaagcttttgtatcttggatcccatgatgttgtccacatcatagggatccatggaatgggcgggttaggaaaaacaacacttgctcgggcagtttataatttgattgctctccattttgatgaatcatgttttcttcaaaacgtgagagaagaatcaaataaacatgggttaaaacaccttcaaagcacccttctttcaaaattacttggtgaaaaggacatcaccttaacaagttggcaagaaggagcttcaatgatacaacataggctccgaagaaagaagattctcttgattttagatgatgttgacaaggacgagcaattaaaggctattgttggaagacctgattggtttggtcccggtagcagagtcatcattaccactcgggacaaacatctgctaaaatgtcacgaggttgaaataacttttgaggtgaatgatttgaattacaaagctgtaacaaaatgggaatctgctgttttatatttttataaaagaattcccagtgatgaaatccaagagatactaaaagtaagctttgatgctttggaggaagaacaaaagaatgtttttcttgatattgcttatttcttgaaagggtataaatggaaagaggttgatgatatactccgtgctctttatggtaactgcaagaagcatcatattggggagttggttgaaaaatctctcataaagcttaactgctatgatggtggtactgttgaaatgcacgacctgcttcaggacatgggtagagaaattgggcggcagagatcaccagaagagccctggaagtgcaagagattatggtcaccaaaagatataattcaagttttaaaacacaacacgggaactagtaaaattgaaatcatatgtctggattgctccatatctgagaaagaagaaacagtggaatggaatgaaaacgccttcatgaagatggaaaaccttaaaatacttattattagaaatggcaaattttccaaagctcccagttattttccagaaggtttgagagtactggaatggcacagatatccttcaaattgtttaccatctaactttaatccgaacaaccttgttatatgcaacttacctgacagttccatgacgtcatttgagttccatggctcatcgaaggcatgtttaaagagtatattttcctcattgcatggctcatcgaagaagctggggcatctaacagttttgaattttgactggtgcaaatttttaacccagatacctgatgtatctgatcttccaaatttgagggaactttcatttcgatggtgtgagagtttagttgcagttgatgagtcaattggttttctgaagaaacttaaaatattgaatgctgctggttgcaggaagcttacgagttttccgcctctcaacttgacctctcttgaaacacttgaactttctcactgttccagtcttgagtattttccagaaatattaggagagatggaaaacataaaggcacttcatttggaaggccttcccataaaagaattgccattttcatttcaaaatcttattggactccgtgagataaccctgaggaggtgtagaattgttcggttacgatgtagcttagccatgatgcccaatctgtttcgattccaaattagaaattgcaacagctggcaataagtagaatcggaagcaggtgaagaaaaagtccctccttcctacaaaccatagctttggggacacgcaagttttccgtagcttttttcttatttttttcatgttttttattttattattttggtttcagttgtcaaaaattcacttaacccaactgatgttcaccctatcttcatcacgtacttttgattcatttatattcatctttcacgactttatgcattttctatatataacctttcaagttttatctttctatatatttttatagtta

SEQ ID NO: 6 - DNA, 2279 bp, Glycine max
  source 1..2279: mol_type = genomic DNA
atgctggtgccccacttaacattttctatactatctttttcatatttgttcaagtttgattctacactcccaccatatcaattaaccattatcatactaaacttgtttcttctaataatggctgcaacgacacgttcccttgcatccatctatgatgtgttcctcaacttcagaggggaagacacgcgctatggttttactggcaatctctacaaggctctttgtgacaagggatttcataccttctttgacgaagacaagcttcacagcggagaggaaataacacctgcacttttgaaggcaattcaagattccaggattgctattattgtgctttctgaaaactatgctttttcctcattttgtttagatgaacttgtagccatcctacactgcaagagggaagggctgttggttataccggtcttttacaaagtagatccttctgatgttagacaccagaaaggtagttatggagaagcaatgactaagcatcacgaaaggttcaaagataagatggagaagctgcagaaatggaggatggcattgcatcaagtagctgacttgtctggcaaacatttcaaagatgggtatacaatcatactaatatattttactttatggtttttattggattatggtttacttgtctattgatttaacttgtaaaatataaagagaaaataaatgcttgttaatcttgacaattaatgtacctatcaagacatggatgcaaggcttttaggctgacttcatcagaactttttttgtttgtttgaaacagagatgcatatgaatacaagtttattgggagtattgttgaggagctctctaggaagattaatcgtgcttctttacatgttgcggattatcctgttggtttagagtcacaagtgacagaggtaatgaagcttttggatgttggatccgatgatgttgtccacatcatagggatccatggaatgggcgggttaggaaaaacaacacttgctctggcagtatataatttcattgctctccattttgatgaatcctgttttcttcaaaacgtgagagaagaatcaaataaacatgggttaaaacaccttcaaagcatccttctttcaaaattacttggtgagaaggacatcatcttaacaagttggcaagaaggagcttcaatgatacaacataggctccgacgaaagaaggttctcttgattttagacgatgttgacaagtgtgagcaattaatggctattgttggaagacctgattggtttggtcccggtagcagagtcatcattaccactcgggacgaacatctgctaaaatgtcacgaggttgaaagaacttatgaggtgaaggttttgaatcacaatgctgctcttcaactgcttactaggaatgcttttaaaagagaaaaaattgatccaagttatgaggacgtcttgaatcgtgtagtagcttatgcttctggccttccattggctttggaagtcataggctcggacttgtttggaaaaactgtagcagaatgggaatctgctgtggaacattataaaagaattcccagggataaaatccaagagatactaaaagtaagctttgatgctttgggggaagaacaaaagaatgtgtttcttgacatcgcttgttgcttcaaagggtataaatggacagaggttgatgatctactccatgttctttatggtaactgcaagaagcatcatattggggtgttggttgaaaaatctctcataaagtactatgatgatactgttgaaatgcacgacctgattcaggacatgggtagagaaattgagcggcagagatcaccagatgagccagggaagcgcaagagattatggtcaccacaagatataattcaagttttaaaacacaacacggtgagtgagctcatgaatagttgaatttttttttgtctatctcatatatttacatcatggttaactttttctttataattcgtaatattttctaagcgagtcaattgatatttcacacaagttgaattgtggttgattcgtgcctttttgcattagcagggaacttgtaaaattgaaatcatatgtttggatccctccatatctgacaaagaagaaacagtggaatggaatgaaaacgccttcatgaagatggaaaaccttaaaatacttattattagaaatggcaatttttcgataggtcccaataattttccagaaggtttgagagtactggaatggcaaagatatccttcaaattgtttaccgtctaactttgatccgatcaactga

SEQ ID NO: 7 - protein, 641 aa, Glycine max
MLVPHLTFSILSFSYLFKFDSTLPPYQLTIIILNLFLLIMAATTRSLASIYDVFLNFRGEDTRYGFTGNLYKALCDKGFHTFFDEDKLHSGEEITPALLKAIQDSRIAIIVLSENYAFSSFCLDELVAILHCKREGLLVIPVFYKVDPSDVRHQKGSYGEAMTKHHERFKDKMEKLQKWRMALHQVADLSGKHFKDGDAYEYKFIGSIVEELSRKINRASLHVADYPVGLESQVTEVMKLLDVGSDDVVHIIGIHGMGGLGKTTLALAVYNFIALHFDESCFLQNVREESNKHGLKHLQSILLSKLLGEKDIILTSWQEGASMIQHRLRRKKVLLILDDVDKCEQLMAIVGRPDWFGPGSRVIITTRDEHLLKCHEVERTYEVKVLNHNAALQLLTRNAFKREKIDPSYEDVLNRVVAYASGLPLALEVIGSDLFGKTVAEWESAVEHYKRIPRDKIQEILKVSFDALGEEQKNVFLDIACCFKGYKWTEVDDLLHVLYGNCKKHHIGVLVEKSLIKYYDDTVEMHDLIQDMGREIERQRSPDEPGKRKRLWSPQDIIQVLKHNTGTCKIEIICLDPSISDKEETVEWNENAFMKMENLKILIIRNGNFSIGPNNFPEGLRVLEWQRYPSNCLPSNFDPIN

SEQ ID NO: 8 - DNA, 4410 bp, Glycine max
  source 1..4410: mol_type = genomic DNA
ttttaatttatctgtgttaacttctttctttacatagaatttattagtgttaactaagaactgcatatatgctcaaagacatacacatgtcctcatatattaaaatccttccttgttattaggggtggaaataggtcaggccaaatcgctttgaaaggtgcttaacctatgatgaatctttgaggccagaacttgacctatagcctatcaacgacttttattttggctcggtctgacggtcttttataagcctggtctgacctgatagtctttttaaatcttcacaataaatatttaatatttcgtggagtagaaacgtaataggaaagttaaagaaaaaggaaagtcaatcaaaataataatgaacaatataaaatgacacatgactcacacctaaattgtaattaaagtcaactttgataagatgtgtacgcgttactagttattgtcgttattatcgtttagcctcatatgtcattgtcactctgcctaataataaacatttgtgtgaccgcgtcaaataatgctggtgccccacttaacattttctatactatctttttcatatttgttcaagtttgattctacactcccaccatatcaattaaccattatcatactaaacttgtttcttctaataatggctgcaacgacacgttcccttgcatccatctatgatgtgttcctcaacttcagaggggaagacacgcgctatggttttactggcaatctctacaaggctctttgtgacaagggatttcataccttctttgacgaagacaagcttcacagcggagaggaaataacacctgcacttttgaaggcaattcaagattccaggattgctattattgtgctttctgaaaactatgctttttcctcattttgtttagatgaacttgtagccatcctacactgcaagagggaagggctgttggttataccggtcttttacaaagtagatccttctgatgttagacaccagaaaggtagttatggagaagcaatgactaagcatcacgaaaggttcaaagataagatggagaagctgcagaaatggaggatggcattgcatcaagtagctgacttgtctggcaaacatttcaaagatggagatgcatatgaatacaagtttattgggagtattgttgaggagctctctaggaagattaatcgtgcttctttacatgttgcggattatcctgttggtttagagtcacaagtgacagaggtaatgaagcttttggatgttggatccgatgatgttgtccacatcatagggatccatggaatgggcgggttaggaaaaacaacacttgctctggcagtatataatttcattgctctccattttgatgaatcctgttttcttcaaaacgtgagagaagaatcaaataaacatgggttaaaacaccttcaaagcatccttctttcaaaattacttggtgagaaggacatcatcttaacaagttggcaagaaggagcttcaatgatacaacataggctccgacgaaagaaggttctcttgattttagacgatgttgacaagtgtgagcaattaatggctattgttggaagacctgattggtttggtcccggtagcagagtcatcattaccactcgggacgaacatctgctaaaatgtcacgaggttgaaagaacttatgaggtgaaggttttgaatcacaatgctgctcttcaactgcttactaggaatgcttttaaaagagaaaaaattgatccaagttatgaggacgtcttgaatcgtgtagtagcttatgcttctggccttccattggctttggaagtcataggctcggacttgtttggaaaaactgtagcagaatgggaatctgctgtggaacattataaaagaattcccagggataaaatccaagagatactaaaagtaagctttgatgctttgggggaagaacaaaagaatgtgtttcttgacatcgcttgttgcttcaaagggtataaatggacagaggttgatgatctactccatgttctttatggtaactgcaagaagcatcatattggggtgttggttgaaaaatctctcataaagtactatgatgatactgttgaaatgcacgacctgattcaggacatgggtagagaaattgagcggcagagatcaccagatgagccagggaagcgcaagagattatggtcaccacaagatataattcaagttttaaaacacaacacgggaacttgtaaaattgaaatcatatgtttggatccctccatatctgacaaagaagaaacagtggaatggaatgaaaacgccttcatgaagatggaaaaccttaaaatacttattattagaaatggcaatttttcgataggtcccaataattttccagaaggtttgagagtactggaatggcaaagatatccttcaaattgtttaccgtctaactttgatccgatcaactgataggaacacagagaagacaacaacaaaggcttacacaggggtcgaaatagcagaacaggaggtgcagaagcaagccaaacccaaatggagaattacgaaaccatcttacctcagtgagtatgtctgaggaagaatggtaaatgaatagcggaaagcaccaaattaggaaaagggaacatccttgctgagtgtgtcttcattagggggacagcactaacagaattacaattgccaaaccattctagaagctttttctgttactagggaaacttatatatatatatgtaaataacacagcaaggaattaatgggaaatacttcaattaccttctttctctgtgccttcttcttcttcttctgtgtcttcttcttctcattttcaggggagtgctaacatcaaccttgttatatgcaagttacctgatagttccattacgtcatttgagttccatggctcatccaagaagttggggcatctaacagttctgaattttgacaagtgcaaatttttaacacagatacctgatgtatctgatcttccaaatttgagggaactttcatttcgatggtgtgagagtttagttgcagttgatgactcaattggttttctgaataaacttaaagaattgagtgctgatggttgcaggaagcttacgagttttccgcctctcaacttgacctctcttgaaacacttgaaatttctgagtgttccagtcttagtattttccagaaatattaggagagatggaaaacatacagaaacttcgtttgactgaccttcccataaaaaaattgccattttcaattcaaaatcttactggactccaatggttaaccctggggagctgtggaattgttcagttaccatgtagcttatccatgatgcccaaactgttggaattcaatattagagattgcaacaggtggcaatgggtagaatcggaagaaggtgaagaaaaagtgggcccaacacagtcttcaacggaacattgctttggtgccacgaagtgcaatttatgtgatgatttttttttaacaggttccaagaggtttgctcttgtaacatttttagatctatcggagaataatttcaccatccttcccgaattcttcaaagaattggaatttttacaaactcttttcgtgagtgattgcgagcatcttcaggaaattagagggcttccaccaaacttagagtattttgatgcaagaaactgtgcatccttgacttcctcgactaaaagcatgcttttaaatcaggaactgcatgaggctagaccaacccagtttgtgtttccaggaacaagaataccagagtggttcgatcagcaaaagagtggacattcaatttctttctggtttcgtaataaactcccagccaaacttctttgtcttcttattgcactgtctactgagaattttatcctatctccagacgtgttcatcaatggaaaatttgaagaattggagccctataagacggacgaaatagaaagtatgtcgaaattggatcatacttatatcttttatctccaaaagttacctttcaaaaatggtaatctgtttgaagaagtggctagggaaaaggaatggaaccatgtggaggttagatatgaaagtgtgttagagttagagagctcactcatcaaaggaagtggaatccatatattcagagaagaaggcagtatggaggaagatattcgatttgatgatccatatctcagcagctctgcatcagaaagcccctccttcctacaaaccatagctttggggacacgcaagttttccattgcttttttcttatttttttcatgtttttttattttattattttggtttcagtcttcaaaaaacgacttaatccaactggtgttcgccctatcttcataatgtacttttgactcatttatattcatctttcatgactttatgcattttctatatataacctttcaagttt

SEQ ID NO: 9 - DNA, 558 bp, Glycine max
  source 1..558: mol_type = genomic DNA
atgaagcttttggatgttggatccgatgatgttgtccatataatagggatccatgggatgcgtgggttaggaaaaacaacccttgctctagctgtttataattcgattgctggtcattttgatgaagcctgttttcttgaaaacgtgagagaagaatcaagtaaacatgggttaaaacaccttcaaagcatcattatttcaaaattacttggtgagaaggacatcaacttagcaagttggcaagaaggagcttcaatgatacaaagtaggctccgacgaaagaaggttctcttgattttagacgatgtcaacaagcgcgagcaattaaaggctattgttggaagatctgattggtttggtcccggtagcagagtcatcattaccactcgggacaaatatctgctaaaagatcacgaggttgaaagaacttatggggtgaattataaattgtttggaaaaactgtagcaaaatgggaatctgctgtcggtgattatacaaagctcttgatatataagacaggggaatctacactttcaatgatagaaggatggctttga

SEQ ID NO: 10 - protein, 185 aa, Glycine max
MKLLDVGSDDVVHIIGIHGMRGLGKTTLALAVYNSIAGHFDEACFLENVREESSKHGLKHLQSIIISKLLGEKDINLASWQEGASMIQSRLRRKKVLLILDDVNKREQLKAIVGRSDWFGPGSRVIITTRDKYLLKDHEVERTYGVNYKLFGKTVAKWESAVGDYTKLLIYKTGESTLSMIEGWL

SEQ ID NO: 11 - DNA, 1993 bp, Glycine max
  source 1..1993: mol_type = genomic DNA
tcaagtttgattctccctcctaccatatcaaccataaccatactaaactttgttcttctaataatggctgcaacgacacgttcccttgcatccatctatgatgtgttcctcagcttcagagggacagacacacgctatggttttactggcaatctctacaaggctctttgtgacaagggatttcataccttctttgacgaagacaagcttcacagcggagaggaaataacacctgcacttttgaaggcaattcaagattccagggttgctattattgtgctttctgaaaactatgctttttcctcattttgtttagatgaacttgtaaccatctttcactgcaagagggaagggctgttggttataccggtcttttacaaagtcgatccttcttatgtcagacaccagaaaggtagttatggagaagcaatgactaagcatcaggaaaggttcaaagataagatggagaagctgcaggaatggaggatggctttgaaacaagtagctgacttgtctggctctcatttcaaagatggagacgcatatgaatacaagtttattgtgaatattgttgaggaggtctctaggaagattggtcgtggttctttacatgttgcggattatccggttggtcaagcgtcacaagtgacagaggtaatgaagcttttggatgttggatccgatgatgttgtccatataatagggatccatgggatgcgtgggttaggaaaaacaacccttgctctagctgtttataattcgattgctggtcattttgatgaagcctgttttcttgaaaacgtgagagaagaatcaagtaaacatgggttaaaacaccttcaaagcatcattatttcaaaattacttggtgagaaggacatcaacttagcaagttggcaagaaggagcttcaatgatacaaagtaggctccgacgaaagaaggttctcttgattttagacgatgtcaacaagcgcgagcaattaaaggctattgttggaagatctgattggtttggtcccggtagcagagtcatcattaccactcgggacaaatatctgctaaaagatcacgaggttgaaagaacttatggggtgaattataaattgtttggaaaaactgtagcaaaatgggaatctgctgtcggtgattatacaaagctcttgatatataagacaggggaatctacactttcaatgatagaaggatggctttgaaacaagtagctgacttgtctggctctcatttcaaagatgggtatacaatcatactaatatattttactttatggtttttattggattaggttttacttgtctattgatttaactagtcaaatttaaatagaaaagaaatgctcttgttaaccttgacaattaatgtacctatcaagacatggatgcaaggattttaggctgacttcatcattaccactcgggacaaatatctgctaaaagatcacgaggttgaaagaacttatggggtgaattataaattgtttggaaaaactgtagcaaaatgggaatctgctgtcggtgattatacaaagctcttgatatatatgacaggggaatctacactttcattgatgatgaggagcttccgagaggagacaaaataacacctgcacttttcagttttttaaatcctcaacgtcttcgtaatttctgtatctttttgcttttaattctttcagtttttgtatttgcagttcataatttgaaaagaaaacatcacttcctaataattaagcatgaataactatgaataactgctaaataatttttttaaagacattttcactttatttttcattatgaaaaacacatttatttaacagttattaatgcataggcctttctccgcaaattgttaggatcccccagttgtaggtttgaatcttaagctgctcttttttgaaacctgtaagagtttaagatcagaaaagattaccatatgtataccattagaataaatttcatcttaattcatatt

SEQ ID NO: 12 - DNA, 20 bp, Glycine max
  source 1..20: mol_type = other DNA
tgtgaacattcgtagttgtc

SEQ ID NO: 13 - DNA, 20 bp, Glycine max
  source 1..20: mol_type = other DNA
ttccactgactcacaaaaag

SEQ ID NO: 14 - DNA, 20 bp, Glycine max
  source 1..20: mol_type = other DNA
tggctcatcgaaggcatgtt

SEQ ID NO: 15 - DNA, 20 bp, Glycine max
  source 1..20: mol_type = other DNA
gaggtcaagttgagaggcgg

SEQ ID NO: 16 - DNA, 22 bp, Glycine max
  source 1..22: mol_type = other DNA
tcccagggataaaatccaagag

SEQ ID NO: 17 - DNA, 20 bp, Glycine max
  source 1..20: mol_type = other DNA
ggtgatctctgccgctcaat

SEQ ID NO: 18 - DNA, 24 bp, Glycine max
  source 1..24: mol_type = other DNA
cactttcaatgatagaaggatggc

SEQ ID NO: 19 - DNA, 20 bp, Glycine max
  source 1..20: mol_type = other DNA
tcctctcggaagctcctcat

SEQ ID NO: 20 - DNA, 22 bp, Glycine max
  source 1..22: mol_type = other DNA
gcgtggcacccttgataaataa

SEQ ID NO: 21 - DNA, 24 bp, Glycine max
  source 1..24: mol_type = other DNA
gcgcacgaaagtttttctgtaaca

SEQ ID NO: 22 - DNA, 20 bp, Glycine max
  source 1..20: mol_type = genomic DNA
ttatgaccaatttttccccc

SEQ ID NO: 23 - DNA, 20 bp, Glycine max
  source 1..20: mol_type = other DNA
gaaattttgactgccacggt

SEQ ID NO: 24 - DNA, 20 bp, Glycine max
  source 1..20: mol_type = other DNA
tgccatatgcaaattaccca

SEQ ID NO: 25 - DNA, 21 bp, Glycine max
  source 1..21: mol_type = other DNA
aaacattcagtcccgtcaaaa

SEQ ID NO: 26 - DNA, 20 bp, Glycine max
  source 1..20: mol_type = other DNA
ccactggtaaatttgggtca

SEQ ID NO: 27 - DNA, 25 bp, Glycine max
  source 1..25: mol_type = other DNA
caaccatgttgttaatatgaatgga

SEQ ID NO: 28 - DNA, 20 bp, Glycine max
  source 1..20: mol_type = other DNA
ccctcttcatttcccttggt

SEQ ID NO: 29 - DNA, 22 bp, Glycine max
  source 1..22: mol_type = other DNA
cctcccaatatctttgggataa

SEQ ID NO: 30 - DNA, 21 bp, Glycine max
  source 1..21: mol_type = other DNA
gcacataacatcacccactga

SEQ ID NO: 31 - DNA, 20 bp, Glycine max
  source 1..20: mol_type = other DNA
aaacagagaggcccgaagtt

SEQ ID NO: 32 - DNA, 26 bp, Glycine max
  source 1..26: mol_type = other DNA
gcgtcttagtcgtgctgacactactc

SEQ ID NO: 33 - DNA, 30 bp, Glycine max
  source 1..30: mol_type = other DNA
gcgagtttgtttagctcaatctttcactca

SEQ ID NO: 34 - DNA, 20 bp, Glycine max
  source 1..20: mol_type = other DNA
tgtgttcttgaatgaggcga

SEQ ID NO: 35 - DNA, 21 bp, Glycine max
  source 1..21: mol_type = other DNA
tcagctgatgtttgaacgaaa

SEQ ID NO: 36 - DNA, 203 bp, Glycine max
  source 1..203: mol_type = other DNA
ttaaaataaaattcaagatgctttatatttgcggaaagtttgatagtctttgaactccggattttctggcgttgatttgtgaacacgatataccctttgccaatgaatttccagcaacttcaaattaacgtaaactttaaatgtttgagaatgtctgtggtagtcttaattgtgatggagataatttttatggagacacgtgc

SEQ ID NO: 37 - DNA, 203 bp, Glycine max
  source 1..203: mol_type = other DNA
ttaaaataaaattcaagatgctttatatttgcggaaagtttgatagtctttgaactccggattttctggcgttgatttgtgaacacgatataccctttgccgatgaatttccagcaacttcaaattaacgtaaactttaaatgtttgagaatgtctgtggtagtcttaattgtgatggagataatttttatggagacacgtgc

SEQ ID NO: 38 - DNA, 203 bp, Glycine max
  source 1..203: mol_type = other DNA
aggtagcctatgttatggataaatttatgttatcccgcgtaatactcagtgttatgggcagttcttttgggagaagaaaaaattgagatatgtttaatctctacttctggaattcaaatttcagccttatatcaaaacctgttaaagtttcacaaccagatgtttcaaaggcattgaatatgatttcgtatatcagtggctat

SEQ ID NO: 39 - DNA, 203 bp, Glycine max
  source 1..203: mol_type = other DNA
aggtagcctatgttatggataaatttatgttatcccgcgtaatactcagtgttatgggcagttcttttgggagaagaaaaaattgagatatgtttaatctcaacttctggaattcaaatttcagccttatatcaaaacctgttaaagtttcacaaccagatgtttcaaaggcattgaatatgatttcgtatatcagtggctat

SEQ ID NO: 40 - DNA, 203 bp, Glycine max
  source 1..203: mol_type = other DNA
ttattccccgacagatttaaatgtcctacacgagcaaaggtcttgaaacctgttaaaaaaaaatcatcacatagattgcaatccttggccgaaaaccgatgtgcctttgaagatggtattgagcccacttttttttcaccttcttccgattctacccattgccacctgttgcaatattccatatggaattcaaacagttcggg

SEQ ID NO: 41 - DNA, 203 bp, Glycine max
  source 1..203: mol_type = other DNA
ttattccccgacagatttaaatgtcctacacgagcaaaggtcttgaaacctgttaaaaaaaaatcatcacatagattgcaatccttggccgaaaaccgatgcgcctttgaagatggtattgagcccacttttttttcaccttcttccgattctacccattgccacctgttgcaatattccatatggaattcaaacagttcggg

SEQ ID NO: 42 - DNA, 203 bp, Glycine max
  source 1..203: mol_type = other DNA
ctctgttttgccgaggagagtgtcactgcaagcataactggtacgacggagccatcagaaccaccatatagcattttgtctttttctcttccttcctccccgctctttctcctttccttctctttcttaccttctccgttgatgatggatttcagatcttcaattgcttcttgcaccaccatcaaccacgactttcgcatcta

SEQ ID NO: 43 - DNA, 203 bp, Glycine max
  source 1..203: mol_type = other DNA
ctctgttttgccgaggagagtgtcactgcaagcataactggtacgacggagccatcagaaccaccatatagcattttgtctttttctcttccttcctccccactctttctcctttccttctctttcttaccttctccgttgatgatggatttcagatcttcaattgcttcttgcaccaccatcaaccacgactttcgcatcta

SEQ ID NO: 44 - DNA, 203 bp, Glycine max
  source 1..203: mol_type = other DNA
tggaagtgaagaaataaagatattttttagatgcaagaaggaggaaactgaaaacgaagcaagtagtactactgtggtgtggtgttattttgtttggttgtttgataataattgtaagcatcagtaatgggcagtgggtggcagcacatggccgccgtgctccaccaatcaccaatccaacttcgctttcttaaatcatatcg

SEQ ID NO: 45 - DNA, 204 bp, Glycine max
  source 1..204: mol_type = other DNA
tggaagtgaagaaataaagatattttttagatgcaagaaggaggaaactgaaaacgaagcaagtagtactactgtggtgtggtgttattttgtttggttgtcctgataataattgtaagcatcagtaatgggcagtgggtggcagcacatggccgccgtgctccaccaatcaccaatccaacttcgctttcttaaatcatatcg

SEQ ID NO: 46 - DNA, 203 bp, Glycine max
  source 1..203: mol_type = other DNA
atataattaaattattttacaagaaatttatatccctatttttttataaaaataatccaaaatcatcaatttcttcccttcccaatgtactgttgacatccctaatatattttcaagtatttttcagtaactactgaattttttgtttcacataacaaattaaaccacactgaaactctggcatgattatatggaattattta

SEQ ID NO: 47 - DNA, 203 bp, Glycine max
  source 1..203: mol_type = other DNA
atataattaaattattttacaagaaatttatatccctatttttttataaaaataatccaaaatcatcaatttcttcccttcccaatgtactgttgacatccttaatatattttcaagtatttttcagtaactactgaattttttgtttcacataacaaattaaaccacactgaaactctggcatgattatatggaattattta

SEQ ID NO: 48 - DNA, 203 bp, Glycine max
  source 1..203: mol_type = other DNA
atccattggaaacaaataagctttgtgttcatgttcgttttcctgaatatttttctagatctccgcagtgaaacattccgattgttgttgagtagaacaccccgagtacgaaaatcgttctcgaagttagtttttcgtgttgagacgaaatcagaaacgaagccaagggcatattgggcataatgttttttaatttttaatta

SEQ ID NO: 49 - DNA, 203 bp, Glycine max
  source 1..203: mol_type = other DNA
atccattggaaacaaataagctttgtgttcatgttcgttttcctgaatatttttctagatctccgcagtgaaacattccgattgttgttgagtagaacaccgcgagtacgaaaatcgttctcgaagttagtttttcgtgttgagacgaaatcagaaacgaagccaagggcatattgggcataatgttttttaatttttaatta

SEQ ID NO: 50 - DNA, 203 bp, Glycine max
  source 1..203: mol_type = other DNA
tgcactttttttttttttttgaaagaaagggtctccctaattatttttgattgtaaacttagccattcattgagctgaaataaagtatcttcaatcattggtgaaaaaggcaaagaatataaattatgaatagaaagcctcgttaaccccgcttcacgggattccattttatgtcaggtatgacaaattaaactgcgttcagt

SEQ ID NO: 51 - DNA, 203 bp, Glycine max
  source 1..203: mol_type = other DNA
tgcactttttttttttttttgaaagaaagggtctccctaattatttttgattgtaaacttagccattcattgagctgaaataaagtatcttcaatcattggagaaaaaggcaaagaatataaattatgaatagaaagcctcgttaaccccgcttcacgggattccattttatgtcaggtatgacaaattaaactgcgttcagt

SEQ ID NO: 52 - DNA, 203 bp, Glycine max
  source 1..203: mol_type = other DNA
aattgaataaagaaaaacaaaaaaattgcaaataagttgaaggatatataattgaaagcggcaggacgccgtttggtggagacccaccaatatgatgtagccctcaggtcattagtggcaacaactccattgttatttgttaccacaacctagctatatcttaatagcctcaatgtccccatccttaggcttcaagagtacta

SEQ ID NO: 53 - DNA, 203 bp, Glycine max
  source 1..203: mol_type = other DNA
aattgaataaagaaaaacaaaaaaattgcaaataagttgaaggatatataattgaaagcggcaggacgccgtttggtggagacccaccaatatgatgtagctctcaggtcattagtggcaacaactccattgttatttgttaccacaacctagctatatcttaatagcctcaatgtccccatccttaggcttcaagagtacta

SEQ ID NO: 54 - DNA, 203 bp, Glycine max
  source 1..203: mol_type = other DNA
tttatggtttgagtactttccagtaatttttgggattatctgttgaaatatacttatctttttttctcatcttttattctgacaaaattatcagaattagcaataacaacttctggattggttctcgttgatgacaaatgtaactaacccactagtattttttttgttctaactcagagccacaaaagttctccaaagatggt

SEQ ID NO: 55 - DNA, 203 bp, Glycine max
  source 1..203: mol_type = other DNA
tttatggtttgagtactttccagtaatttttgggattatctgttgaaatatacttatctttttttctcatcttttattctgacaaaattatcagaattagcgataacaacttctggattggttctcgttgatgacaaatgtaactaacccactagtattttttttgttctaactcagagccacaaaagttctccaaagatggt

SEQ ID NO: 56 - DNA, 203 bp, Glycine max
  source 1..203: mol_type = other DNA
cactgtaagtaatacagcataacttgaaaaatgttcaaagagtactaggaaaatggctactgaaggtactttgagtttgagggtcatgccgcctcccactccaaatctttccctcgaaactgtgtccctccatttctttctcttccttccctgttaaagttcccccacttacaacctgctgcatagacagacaattacaatta

SEQ ID NO: 57 - DNA, 203 bp, Glycine max
  source 1..203: mol_type = other DNA
cactgtaagtaatacagcataacttgaaaaatgttcaaagagtactaggaaaatggctactgaaggtactttgagtttgagggtcatgccgcctcccactctaaatctttccctcgaaactgtgtccctccatttctttctcttccttccctgttaaagttcccccacttacaacctgctgcatagacagacaattacaatta

SEQ ID NO: 58 - DNA, 203 bp, Glycine max
  source 1..203: mol_type = other DNA
caaaatttttactaaaaatcacaatatagtttgtagtactttttttcataattttttttccacttttgattccttttaatatgcactaaaaaatctaaaatgactttttattcaataaaatgatgaattgtgtttaacaccaataatcatgtttgacactactttcaaagacatcaaacaagtctcctggtgttttcggatgc

SEQ ID NO: 59 - DNA, 203 bp, Glycine max
  source 1..203: mol_type = other DNA
caaaatttttactaaaaatcacaatatagtttgtagtactttttttcataattttttttccacttttgattccttttaatatgcactaaaaaatctaaaataactttttattcaataaaatgatgaattgtgtttaacaccaataatcatgtttgacactactttcaaagacatcaaacaagtctcctggtgttttcggatgc

SEQ ID NO: 60 - DNA, 203 bp, Glycine max
  source 1..203: mol_type = other DNA
atcaacaacataagaagtataacaagagaaccaggagcgttttttttctttgcatgctctgtttgtttggcagagaaaaacagggaagagtgagagaaagtaggaaacttttcaacatcagtacatctcatttgaaaaacaaaacatgcccattaaagatcagatacattgttaaacaatgcagcaagcagcaattacatggc

SEQ ID NO: 61 - DNA, 203 bp, Glycine max
  source 1..203: mol_type = other DNA
atcaacaacataagaagtataacaagagaaccaggagcgttttttttctttgcatgctctgtttgtttggcagagaaaaacagggaagagtgagagaaagttggaaacttttcaacatcagtacatctcatttgaaaaacaaaacatgcccattaaagatcagatacattgttaaacaatgcagcaagcagcaattacatggc

SEQ ID NO: 62 - DNA, 203 bp, Glycine max
  source 1..203: mol_type = other DNA
gaaaaggatacgtgaaagaaagcactaaccaagattatcatagaaattacaagaagaggtccagaccaaagaacagacataaatagtccactcggatcaaagtagttatgactagagaagcttttccagttttttcccaaaaatctgttcagactctcagcaagatatacaccaaccactgcatcaaaacaatttgtaggtaa

SEQ ID NO: 63 - DNA, 203 bp, Glycine max
  source 1..203: mol_type = other DNA
gaaaaggatacgtgaaagaaagcactaaccaagattatcatagaaattacaagaagaggtccagaccaaagaacagacataaatagtccactcggatcaaactagttatgactagagaagcttttccagttttttcccaaaaatctgttcagactctcagcaagatatacaccaaccactgcatcaaaacaatttgtaggtaa

SEQ ID NO: 64 - DNA, 203 bp, Glycine max
  source 1..203: mol_type = other DNA
ttttatatttttttatattctattttaatttaatatttttattttttttatcctctaaattaaacacaccataagggttgaagagaggaacggaaggaatcttgggtttaaaccctcctattaatattcatattctagtaataactaacaatgattgtatttattgaataaatgaaacaattaataatcaagagaagtcattt

SEQ ID NO: 65 - DNA, 203 bp, Glycine max
  source 1..203: mol_type = other DNA
ttttatatttttttatattctattttaatttaatatttttattttttttatcctctaaattaaacacaccataagggttgaagagaggaacggaaggaatcgtgggtttaaaccctcctattaatattcatattctagtaataactaacaatgattgtatttattgaataaatgaaacaattaataatcaagagaagtcattt

SEQ ID NO: 66 - DNA, 20 bp, Glycine max
  source 1..20: mol_type = other DNA
aaaaatcagcacccatcgac

SEQ ID NO: 67 - DNA, 20 bp, Glycine max
  source 1..20: mol_type = other DNA
agccctggccttattttgtt

SEQ ID NO: 68 - DNA, 20 bp, Glycine max
  source 1..20: mol_type = other DNA
ctctcctttcattccccaca

SEQ ID NO: 69 - DNA, 20 bp, Glycine max
  source 1..20: mol_type = other DNA
ttcttggagcttcggaggta

SEQ ID NO: 70 - DNA, 22 bp, Glycine max
  source 1..22: mol_type = other DNA
gaactccacttaatcatctcac

SEQ ID NO: 71 - DNA, 21 bp, Glycine max
  source 1..21: mol_type = other DNA
ttcactccgtcctcggcggcg

SEQ ID NO: 72 - DNA, 37 bp, Glycine max
  source 1..37: mol_type = other DNA
atttcctaattaagtgaaagtttgaaatgttatatta

SEQ ID NO: 73 - DNA, 28 bp, Glycine max
  source 1..28: mol_type = other DNA
gatttatcacactatcaaagtgtatgac

SEQ ID NO: 74 - DNA, 20 bp, Glycine max
  source 1..20: mol_type = other DNA
tcgcaatattggctacgatg

SEQ ID NO: 75 - DNA, 26 bp, Glycine max
  source 1..26: mol_type = other DNA
ctgaaaacaaaataaaagagaacaaa

SEQ ID NO: 76 - DNA, 18 bp, Glycine max
  source 1..18: mol_type = other DNA
ctctgtccccacctctcc

SEQ ID NO: 77 - DNA, 18 bp, Glycine max
  source 1..18: mol_type = other DNA
catggtcagtttgatagc

SEQ ID NO: 78 - DNA, 21 bp, Glycine max
  source 1..21: mol_type = other DNA
taagtgattcgtttgagtcct

SEQ ID NO: 79 - DNA, 22 bp, Glycine max
  source 1..22: mol_type = other DNA
tatggtgtggctatggagattg

SEQ ID NO: 80 - DNA, 21 bp, Glycine max
  source 1..21: mol_type = other DNA
gcatcaacacttggcgcaagc

SEQ ID NO: 81 - DNA, 24 bp, Glycine max
  source 1..24: mol_type = other DNA
ggataatgcgataattgttctagc

SEQ ID NO: 82 - DNA, 20 bp, Glycine max
  source 1..20: mol_type = other DNA
aaatatagcaccctttagag

SEQ ID NO: 83 - DNA, 17 bp, Glycine max
  source 1..17: mol_type = other DNA
agcctcactctccacat

SEQ ID NO: 84 - DNA, 22 bp, Glycine max
  source 1..22: mol_type = other DNA
tttaactgaaaatactccggca

SEQ ID NO: 85 - DNA, 24 bp, Glycine max
  source 1..24: mol_type = other DNA
tcataatttaagagaccaaaccga

SEQ ID NO: 86 - DNA, 23 bp, Glycine max
  source 1..23: mol_type = other DNA
ttggaattctgaaagtgttgttg

SEQ ID NO: 87 - DNA, 20 bp, Glycine max
  source 1..20: mol_type = other DNA
ggaaatccaaccccaaaaat

SEQ ID NO: 88 - DNA, 21 bp, Glycine max
  source 1..21: mol_type = other DNA
gcaaacttgcatcaacttcaa

SEQ ID NO: 89 - DNA, 20 bp, Glycine max
  source 1..20: mol_type = other DNA
tccaccttttccaacattcc

SEQ ID NO: 90 - DNA, 21 bp, Glycine max
  source 1..21: mol_type = other DNA
catattttccgctcactttgc

SEQ ID NO: 91 - DNA, 23 bp, Glycine max
  source 1..23: mol_type = other DNA
aaattagcaaaatgcatgtaccc

SEQ ID NO: 92 - DNA, 20 bp, Glycine max
  source 1..20: mol_type = other DNA
tcgtcgcatgcaacttttta

SEQ ID NO: 93 - DNA, 20 bp, Glycine max
  source 1..20: mol_type = other DNA
ttagctcgcaacaccatcac

SEQ ID NO: 94 - DNA, 20 bp, Glycine max
  source 1..20: mol_type = other DNA
aatgggttggaaacttgcag

SEQ ID NO: 95 - DNA, 22 bp, Glycine max
  source 1..22: mol_type = other DNA
tgggtacgtcaaaattaggaaa

SEQ ID NO: 96 - DNA, 20 bp, Glycine max
  source 1..20: mol_type = other DNA
ttctttgtttgacggtggtg

SEQ ID NO: 97 - DNA, 22 bp, Glycine max
  source 1..22: mol_type = other DNA
tgactcgaacacaaaactcctt

SEQ ID NO: 98 - DNA, 20 bp, Glycine max
  source 1..20: mol_type = other DNA
tggccagaacttgaaggaac

SEQ ID NO: 99 - DNA, 21 bp, Glycine max
  source 1..21: mol_type = other DNA
tgcttattattgcaccccatt

SEQ ID NO: 100 - DNA, 20 bp, Glycine max
  source 1..20: mol_type = other DNA
tgatccctttaacccagcaa

SEQ ID NO: 101 - DNA, 20 bp, Glycine max
  source 1..20: mol_type = other DNA
caatgacaaaaagccaacca

SEQ ID NO: 102 - DNA, 20 bp, Glycine max
  source 1..20: mol_type = other DNA
ttccccgatgaattatttgc

SEQ ID NO: 103 - DNA, 22 bp, Glycine max
  source 1..22: mol_type = other DNA
tgtgataaaaggatccagagca

SEQ ID NO: 104 - DNA, 24 bp, Glycine max
  source 1..24: mol_type = other DNA
ggtagatccaggagcttgagtcag

SEQ ID NO: 105 - DNA, 24 bp, Glycine max
  source 1..24: mol_type = other DNA
gcgcatctcactgcacttgatttt

SEQ ID NO: 106 - DNA, 23 bp, Glycine max
  source 1..23: mol_type = other DNA
ttcttagctgccactatttacga

SEQ ID NO: 107 - DNA, 20 bp, Glycine max
  source 1..20: mol_type = other DNA
tgccatgcatacacatcctt

SEQ ID NO: 108 - DNA, 21 bp, Glycine max
  source 1..21: mol_type = other DNA
ttttgccacctaattgtgtct

SEQ ID NO: 109 - DNA, 20 bp, Glycine max
  source 1..20: mol_type = other DNA
tctcacaacaggtgagtcgg

SEQ ID NO: 110 - DNA, 23 bp, Glycine max
  source 1..23: mol_type = other DNA
ccaacttgaaattactagagaaa

SEQ ID NO: 111 - DNA, 21 bp, Glycine max
  source 1..21: mol_type = other DNA
cttactagcgtattaaccctt

SEQ ID NO: 112 - DNA, 20 bp, Glycine max
  source 1..20: mol_type = other DNA
cccattacaccatgtcacca

SEQ ID NO: 113 - DNA, 23 bp, Glycine max
  source 1..23: mol_type = other DNA
atccgtgttaagggatctaactt

SEQ ID NO: 114 - DNA, 20 bp, Glycine max
  source 1..20: mol_type = other DNA
ccaagtggcctaatttccaa

SEQ ID NO: 115 - DNA, 21 bp, Glycine max
  source 1..21: mol_type = other DNA
ttcaagaagagaaccgtgcat

SEQ ID NO: 116 - DNA, 24 bp, Glycine max
  source 1..24: mol_type = other DNA
gccacttgacactcaaatctactt

SEQ ID NO: 117 - DNA, 23 bp, Glycine max
  source 1..23: mol_type = other DNA
ataacagattatgagttctgcag

SEQ ID NO: 118 - DNA, 21 bp, Glycine max
  source 1..21: mol_type = other DNA
accctctctagaatggtgtgt

SEQ ID NO: 119 - DNA, 22 bp, Glycine max
  source 1..22: mol_type = other DNA
agaagaatgaaaatcacccaat

SEQ ID NO: 120 - DNA, 24 bp, Glycine max
  source 1..24: mol_type = other DNA
gtccactaaaatgacattgtgaaa

SEQ ID NO: 121 - DNA, 24 bp, Glycine max
  source 1..24: mol_type = other DNA
caggtatggatatgtatgtatcaa

SEQ ID NO: 122 - DNA, 21 bp, Glycine max
  source 1..21: mol_type = other DNA
atgaactcatgtgttcaatga

SEQ ID NO: 123 - DNA, 20 bp, Glycine max
  source 1..20: mol_type = other DNA
tgcacacgtttgaattaggg